SEQUENCE LISTING (63 sequences)

SEQ ID NO: 1 - DNA, 573 bp, Artificial Sequence
  Soybean codon optimized OKC1
atgatgaaatctttttttctcgttgtaactattttggctttgactttgccctttcttggagcacaagagcagaatcaagagcagccaatccgttgtgagaaggacgagaggttcttctcagacaagatcgccaaatatatacccatacaatatgtactctcacgctaccctagctacgggcttaactactatcagcaaaaacctgtagcactgataaataaccagtttctcccctatccctattatgctaaacctgccgccgtgaggagtccagcacaaatacttcagtggcaagtgctcagtaacaccgtgccagcaaaaagctgccaggctcagcccaccacaatggcccgtcatccccatcctcaccttagcttcatggcaatcccaccaaagaagaatcaagacaagaccgaaatacctaccatcaacacaattgcatctggagagcctaccagtacaccaacaactgaggcagtagagtctactgttgctacccttgaggacagccccgaggttatagagtccccacctgagataaataccgtgcaggtgacaagtaccgccgtataa

SEQ ID NO: 2 - DNA, 513 bp, Artificial Sequence
  Soybean codon optimized OKC1-T
atgcaagagcagaatcaagagcagccaatccgttgtgagaaggacgagaggttcttctcagacaagatcgccaaatatatacccatacaatatgtactctcacgctaccctagctacgggcttaactactatcagcaaaaacctgtagcactgataaataaccagtttctcccctatccctattatgctaaacctgccgccgtgaggagtccagcacaaatacttcagtggcaagtgctcagtaacaccgtgccagcaaaaagctgccaggctcagcccaccacaatggcccgtcatccccatcctcaccttagcttcatggcaatcccaccaaagaagaatcaagacaagaccgaaatacctaccatcaacacaattgcatctggagagcctaccagtacaccaacaactgaggcagtagagtctactgttgctacccttgaggacagccccgaggttatagagtccccacctgagataaataccgtgcaggtgacaagtaccgccgtataa

SEQ ID NO: 3 - DNA, 675 bp, Artificial Sequence
  Soybean codon optimized OBC1
atgaaggtcttgatattggcatgtctggtagccttggcactggctagagagcttgaggaactcaacgtaccaggggagattgtagaatctctttccagcagcgaggagagtatcacaaggattaataagaaaatcgaaaagttccaaagtgaggaacaacaacaaaccgaagacgaacttcaagacaaaatacacccatttgcacagacacaatcactcgtgtatccattcccaggaccaatcccaaacagtttgcctcaaaacatacctccactgactcaaactccagttgtggtacctcctttcctgcaaccagaagtgatgggtgtctcaaaagtaaagggggcaatggctcctaaacataaagagatgcctttccctaagtatccagtggaaccactgactgagagtcaatcacttaccctcactgatgtagagaaccttcacctccccctgcctttgctgcaaagctggatgcaccaacctcatcagcctcttcctcccaccgttatgtttccccctcagtccgtcctgagtttgagtcagtccaaagtcttgcctgttccccaaaaagcagtgccatacccacaaagggacatgccaatacaagcattcctcctttaccaggagcccgtactcggccctgtgcgtggtcctttccctattatagtctaa

SEQ ID NO: 4 - DNA, 633 bp, Artificial Sequence
  Soybean codon optimized OBC1-T
atgagagagcttgaggaactcaacgtaccaggggagattgtagaatctctttccagcagcgaggagagtatcacaaggattaataagaaaatcgaaaagttccaaagtgaggaacaacaacaaaccgaagacgaacttcaagacaaaatacacccatttgcacagacacaatcactcgtgtatccattcccaggaccaatcccaaacagtttgcctcaaaacatacctccactgactcaaactccagttgtggtacctcctttcctgcaaccagaagtgatgggtgtctcaaaagtaaagggggcaatggctcctaaacataaagagatgcctttccctaagtatccagtggaaccactgactgagagtcaatcacttaccctcactgatgtagagaaccttcacctccccctgcctttgctgcaaagctggatgcaccaacctcatcagcctcttcctcccaccgttatgtttccccctcagtccgtcctgagtttgagtcagtccaaagtcttgcctgttccccaaaaagcagtgccatacccacaaagggacatgccaatacaagcattcctcctttaccaggagcccgtactcggccctgtgcgtggtcctttccctattatagtctaa

SEQ ID NO: 5 - protein, 190 aa, Artificial Sequence
  Soybean codon optimized OKC1
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 6 - protein, 170 aa, Artificial Sequence
  Soybean codon optimized OKC1-T
MQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 7 - protein, 224 aa, Artificial Sequence
  Soybean codon optimized OBC1
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKGAMAPKHKEMPFPKYPVEPLTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 8 - protein, 210 aa, Artificial Sequence
  Soybean codon optimized OBC1-T
MRELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKGAMAPKHKEMPFPKYPVEPLTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 9 - DNA, 645 bp, Artificial Sequence
  Soybean codon optimized OS1C1
atgaaacttctcattcttacctgtctggtggctgtagctctcgcccgcccaaaacatcccataaaacatcaaggattgccccaggaagtactcaacgagaatctcctccgttttttcgttgctcctttccccgaagtgttcgggaaggaaaaagtaaacgagctttcaaaggacatcggctctgaaagtaccgaggatcaggctatggaagatatcaagcaaatggaggccgaatctataagttcttcagaagaaatagttcccaactcagtggagcagaagcacattcagaaagaagacgtgcccagcgagcgctatctgggatatttggaacagctgctcagactgaaaaagtacaaggtgcctcagctcgaaatcgtacccaatagtgctgaagaaaggttgcactcaatgaaagaggggattcacgcacaacaaaaagagcctatgatcggagtaaatcaagaactggcatacttttatcccgagttgtttcgccaattctatcaactggatgcctacccttccggtgcatggtactacgtacccctcggtactcaatataccgatgctccctccttttccgacattcctaatcctataggttccgagaatagcgaaaagaccaccatgcccttatggtaa

SEQ ID NO: 10 - DNA, 669 bp, Artificial Sequence
  Soybean codon optimized OS2C1
atgaagttttttatctttacctgtttgcttgcagtcgccttggccaagaatactatggaacacgtaagctcaagtgaagaatctataataagtcaagagacatataagcaagagaaaaacatggcaataaatccctccaaggagaatctttgtagcactttttgcaaagaagttgtgagaaatgcaaatgaggaagaatactcaataggcagctcttccgaagaatctgctgaagtcgctactgaagaggtcaaaataacagttgacgacaagcattatcaaaaagccctgaatgaaataaaccagttctaccaaaaatttccccaatacctccagtacctttatcaaggacccatagtcctcaacccttgggatcaggtcaagcgtaatgctgttccaataacaccaacactcaatcgtgaacaactgtctacctcagaagaaaattccaaaaaaactgtggatatggaaagtacagaagtttttactaaaaagaccaagctcaccgaggaggaaaaaaatagattgaattttcttaagaagatcagtcaacgctatcagaagttcgcccttccacaatacctcaagactgtataccaacatcagaaggccatgaagccttggattcagcccaaaacaaaggtaatcccctatgttagatacttgtaa

SEQ ID NO: 11 - protein, 214 aa, Artificial Sequence
  Soybean codon optimized OS1C1
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 12 - protein, 222 aa, Artificial Sequence
  Soybean codon optimized OS2C1
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 13 - DNA, 60 bp, Artificial Sequence
  Soybean codon optimized 2A peptide
cagcttctgaattttgatctccttaaacttgctggggacgtggaatctaatcctgggcca

SEQ ID NO: 14 - DNA, 751 bp, Artificial Sequence
  GFP and 6x His-tag
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaaggctagccatcaccatcaccatcacgtgtgaattg

SEQ ID NO: 15 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKDDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 16 - protein, 222 aa, Bos taurus; identical to SEQ ID NO: 12

SEQ ID NO: 17 - protein, 224 aa, Bos taurus; identical to SEQ ID NO: 7

SEQ ID NO: 18 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 19 - DNA, 429 bp, Artificial Sequence
  Soybean codon optimized OLA1 sequence
atgatgagtttcgtttctctcctgctcgtgggtattctttttcatgcaacacaggctgaacaactgaccaagtgcgaagtattcagggaactgaaggaccttaaagggtatggcggcgtgtctctgcctgagtgggtatgtactacttttcatacatcagggtatgacacccaagctattgttcagaacaatgattcaactgaatatggtttgttccagataaataataaaatttggtgcaaagacgaccaaaaccctcacagcagcaacatttgcaacatctcctgtgataaatttctggacgatgacctgaccgacgacatcatgtgtgttaaaaagattctcgataaggtcggtattaactattggctcgctcacaaggcattgtgcagtgagaaacttgatcaatggctctgtgaaaaactttga

SEQ ID NO: 20 - DNA, 537 bp, Artificial Sequence
  Soybean codon optimized OLG1 sequence
atgaaatgtctccttttggcattggcactcacatgcggagcacaagccttgatcgtaacacagactatgaagggtcttgatatacagaaggtggccgggacttggtacagtttggcaatggccgcatccgacatctccttgttggacgcacaatcagccccattgcgtgtgtacgtagaagagcttaaaccaactcccgagggggatctggaaattctgctccagaaatgggagaacggtgagtgcgcccagaagaagatcatcgcagagaagaccaaaattccagcagtattcaaaatcgacgcattgaacgaaaataaggtgctcgtactggacactgattataagaagtatctccttttctgtatggagaactcagcagagcctgaacagagtcttgcctgccaatgccttgttcgtaccccagaggtagatgatgaagctctggaaaagttcgataaggcccttaaggctctgcctatgcacattaggctttctttcaatccaactcaacttgaggaacaatgtcacatttag

SEQ ID NO: 21 - DNA, 447 bp, Artificial Sequence
  Soybean codon optimized OLY1 sequence
atgaaagccctcctgatcgtgggtctgctcctcttgagcgttgcagtacagggtaagaaatttcagcgttgcgaacttgccaggacactgaagaaacttgggctcgacgggtatcgtggagtgtcattggctaactgggtctgtcttgcacgttgggaatccaattacaacactcgtgccaccaactacaaccgtggcgataagtccaccgattatggcatctttcagattaactctcgctggtggtgtaacgatggcaaaacccccaaggcagtaaacgcctgtaggatcccatgttctgctttgctcaaggacgatataacacaggctgtggcttgcgccaagagagtagtacgtgatccccaaggtatcaaggcttgggtcgcttggaggaacaagtgtcagaatagagacttgcgcagctatgtacaaggctgtcgcgtttaa

SEQ ID NO: 22 - protein, 142 aa, Artificial Sequence
  Soybean codon optimized sequence
MMSFVSLLLVGILFHATQAEQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 23 - protein, 178 aa, Artificial Sequence
  Soybean codon optimized sequence
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 24 - protein, 148 aa, Artificial Sequence
  Soybean codon optimized sequence
MKALLIVGLLLLSVAVQGKKFQRCELARTLKKLGLDGYRGVSLANWVCLARWESNYNTRATNYNRGDKSTDYGIFQINSRWWCNDGKTPKAVNACRIPCSALLKDDITQAVACAKRVVRDPQGIKAWVAWRNKCQNRDLRSYVQGCRV

SEQ ID NO: 25 - protein, 142 aa, Bos taurus; identical to SEQ ID NO: 22

SEQ ID NO: 26 - protein, 178 aa, Bos taurus; identical to SEQ ID NO: 23

SEQ ID NO: 27 - protein, 148 aa, Bos taurus; identical to SEQ ID NO: 24

SEQ ID NO: 28 - DNA, 1384 bp, Glycine max
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcacc

SEQ ID NO: 29 - DNA, 57 bp, Glycine max
atggccaagctagttttttccctttgttttctgcttttcagtggctgctgcttcgct

SEQ ID NO: 30 - DNA, 1387 bp, Glycine max
tacattttgagttgtttcaggttccattgccttattgctaaaactccaactaaaataacaaatagcacatgcaggtgcaaacaacacgttactctgatgaaggtgatgtgcctctagcagtctagcttatgaggctcgctgcttatcaacgattcatcattccccaagacgtgtacgcagattaaacaatggacaaaacttcaatcgattatagaataataattttaacagtgccgacttttttctgtaaacaaaaggccagaatcatatcgcacatcatcttgaatgcagtgtcgagtttggaccatttgagtacaaagccaatattgaatgatttttcgattttacatgtgtgaatcagacaaaagtgcatgcaatcacttgcaagtaaattaaggatactaatctattcctttcattttatatgctccacttttatataaaaaaatatacattattatatatgcattattaattattgcagtattatgctattggttttatggccctgctaaataacctaaatgagtctaactattgcatatgaatcaaatgaaggaagaatcatgatctaaacctgagtacccaatgcaataaaatgcgtcctattacctaaacttcaaacacacattgccatcggacgtataaattaatgcatatagattattttgagaaaagaaaacatcaaaagctctaaaacttcttttaactttgaaataagctgataaaaatacgctttaaatcaactgtgtgctgtatataagctgcaatttcacattttaccaaaccgaaacaagaatggtaacagtgaggcaaaaatttgaaaaatgtcctacttcacattcacatcaaattaattacaactaaataaataaacatcgtgattcaagcagtaatgaaagtcgaaatcagatagaatatacacgtttaacatcaattgaatttttttttaaatggatatatacaagtttactattttatatataatgaaaattcattttgtgttagcacaaaacttacagaaagagataaattttaaataaagagaattatatccaattttataatccaaaataatcaaattaaagaatattggctagatagaccggctttttcactgcccctgctggataatgaaaattcatatcaaaacaatacagaagttctagtttaataataaaaaagttggcaaactgtcattccctgttggtttttaagccaaatcacaattcaattacgtatcagaaattaatttaaaccaaatatatagctacgagggaacttcttcagtcattactagctagctcactaatcactatatatacgacatgctacaagtgaagtgaccatatcttaatttcaaatcataaaattcttccaccaagtt

SEQ ID NO: 31 - DNA, 51 bp, Glycine max
atgggtttccttgtgttgcttcttttctccctcttaggtctctcttctagt

SEQ ID NO: 32 - DNA, 1490 bp, Glycine max
attatttctgttagtacatagctaatactcaatcaacggaattagtatatggttcttcatataggagagtacttatttattctattgaattttaacatataagcataataaaatacttttggactctcgtataaagttcgattttaatctttttaataattcaatctaaatgtttaattccctcttaaatgcaaaattcagttttcgttcctttaatgtgacaccattaggtcacatgaaccggaaatgacgtggtgatcgaattatgacttgaatccattgaccacattagcatttcacctatggtcactagtatgaaggatgaaaacaagtctatttctcaaattataaatgaaaacgtttaactttaaacctgaggatccaaaaacgaattttactaaattttgaagaactaaaaaatatttaatctagtaaaacgcgtgtctatctaatataacatgcacgctcgtcatgtaatcaattaggcataaaaatagtgtttgattttttgacacattattaagtgttttatttttaagtttaaaagcattggtatcctttcataaaaggaggtaatcttatttaagtcaaggagaattattatgggaaataaaacctttttttttaaagtgtttaatataattatatactcaaaattcgatttatgattaaatctaagtgacatttaaaaaaaattagtgtgaaaataatttatatataattttgaaaaatttatcattaatttttttttataaataaatgttaatttattagtttttattataaatgtgaatagaatggattcgaagcagcaatttctctctttctccttttccatgccaaccttatatatggtgacgaactgcatatacagtaaaacagttcaaattgagaaagattttaaacatcatagtatttgatatatatcttttacagagacaattatgctgcaggagttagataagattattgtggatgtcattttcttttttaatatttaacgcattatataaaagatgatatagtatggttataaaaaaattatttaacagtttataaaacctttttttttatcttttacagtaatattatttattttatttcacatttttttcatatccttatctcatttataaaggaaattaattgtataaaaaaaatatgatgcactgaatagaatgctgatcttattgtataaggaggatagaatttgagacgcggagaatctgtagagggggaccattcagggtgcctgcaattttggtgttgttcatgtacggttgcagatataaacgaagcatagcttatgtatgaggtgtaacaaaattggaaacaatagccatgcaaggtgaagaatgtcaccaactcagaaacccttcttcattgacgtgtccctcactcactctcctctcttcactataaatcgccactcttcgtgttctccacttcaccaactccttcaaacttattaacactttccttagttcaat

SEQ ID NO: 33 - DNA, 72 bp, Glycine max
atggggaagcccttcttcactctctctctttcttccctttgcttgctactcttgtcgagtgcatgctttgct

SEQ ID NO: 34 - DNA, 1482 bp, Glycine max
aacttaatcgtatataaaaaattcaatatatgaataattctaagtgagtttttaagaaaaaataaaattagtaacgaagtaatttatatataattttgaaaaattatcactaaatttgtgatccactgttaacattaatttattcctcttgtattgaataaaatagttcagacatggtcccagtctttaatcaattattcatgcttctctgtctctcacttatataatcctgtaatccaaacattactcagatagctagatccaccgatcaatcgtatatatatacgcataaaatcgacgcctctgtattttttagactgtagcccaaattcactatccgaataaaataagggaggcacgtgtacgtaatttatatcatatgatagccatgcatatgcacacgtgcagaagagctgttaccctctatacgtgtactcaccttctcatcctctctgaatattttgagtgctcttcctagttatctagtaatgcatgaaattaaacttactaaatgtttcttcaatttaaagaaataattgtttatctgtttcaatttttttaagagaattttaaaaagataattgtttcggggagagagatataaaaaagaaaagggagaaatattaaaatgtactaaataatatgataagaaaagagagaaaaataaaagagaaaatttgtatatagttataattattcatgtaataaggattcatctctcaactgaaaatatacttaatgcagaagaaaaaatcattatttacaaacgttgagtcttgagtgggaaaagaggaggcgccgttactatacaatataagatcatagtactgacaaaatgcacagtaaaacagttcaaattgagaaggattcttaacacaccatagtatttaatatatatctttacagagacaattatgctggaggattcaggcaaagattatatattgtggatttgttttttaataattaacgcatcatatgaaagatcgatgatatatactaatggttataagaaaaatatttaacagtttctataacctttttcttttatcttttactgtaatattatttattttatttcacatttttaatcagcttatctcatttataaacgaaattgtataaaaatatacatgatgaactgaatagaacaatattgatctgatattctcatattgtataagaggatagactttgagacgcggagaatctgtaggaggggaccattcagagtgcctccaattttggtgttgttcattgtaccattgcaaatataaacgaagcatgcatgcttatgtatgaggtgtaacaaaattggaaacaatagccatgcaaggtgaagaatgtcacaaactcagcaacccttattcattgacgtgtccctcagtcactctcctctcatacctataaatcaccactcctcatgttctttccaattaccaactccttcaaacttaattattaacacttccttagttcaat

SEQ ID NO: 35 - DNA, 69 bp, Glycine max
atggggaagcccttcactctctctctttcttccctttgcttgctactcttgtcgagtgcatgctttgct

SEQ ID NO: 36 - DNA, 1594 bp, Glycine max
ataattataaaattgtcactgcgttcaaaacgacaatggttttgggacaactatcattaatcgtgcattgtaaaaaggtgtgtttttagtagtggaccctcgataaattgactgtgatgattgttacatgttgttaagtctcacctataagaaaaaaactaaacatatatatagatcccaattttggggtcaggtgtatagatgaaaaaaagaaacaaatagacaaataaaaaaataaaagaaaaaaaattgatagatgtgagaaatgatgagaagagaagtgcaaataacacactctttctaacattattttactattgattaaaatttattgaaaattactatataatataaaaagtgaaactagttaaactatagtcaataattgagaatatttaaaaatttagaaaatacattacttatatttcttaaaataaaaaatataaataaaaatagaaaaaatggagtaaaatgagatagaagagaagttaggtttataaatacattagttccgcctacaatatatttaaattagctagattaatgcagtaaatttttggcatttacttgattttattttctttaaaagcattctttgtattcttcactgatggttttttttcttcatctgcattatgaattaaatcatttactttgtgtcacaattgcatttagcgaggtcatgcattggttagaccgacggtgtattatgtcatgacttaggtcttgaaggttgttggttacttattatggtccatgggtacacgcgttggttagattcgataggcaaattttgtgaacgatagaaatttatctttattaaataaaccacactatatatatatatatatatatatatatatatatatatatatatattaattcgtaatttcttttctgtctttcattttgattttcttttatggcttttatctttaaaaattttccccttctttaaaatttacaacactttataatcacaataaaataaaataatttaaaatattacataaataataacacaaatatttataaatctgaaatgacataaaataacattataatcacaaaaagtatttaataaaaataaaattacataaataaaatattgtgaaaactaagtaaaaggtatcatgcacgtaatcatatgaaaatagctttagaaaaaatatcaaggcaagtaccgcacgtacgataaatgaaaaaagattaaaaagaaatataataaataataatactaaattaatggtgaataaaatactaaaaaaataaatttataattaaataatatgtattacaaacacaaataagaaataatagtacataatattataataaatagtagtatataacatatcataaatatgtttaaaataatgataaaatattgagtttcttttagtggaactatttgtcaaaatgtgaacacctggatatgaaaaggcatcttaggtagatgatatgatgcgatagaacgtaaaagaaaaatgagaaatgttgatgagaggttaaaaatacccttcataacaagcacacatctataagtagtcttattcacccaacaacgttgcttattcacgcaactaaataagaa

SEQ ID NO: 37 - DNA, 75 bp, Glycine max
atgaagagtactatcttctttgctctctttctagtttgtgccttcaccatctcatacctgccttcagccaccgct

SEQ ID NO: 38 - DNA, 1500 bp, Glycine max
atgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatattgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacatttagctaggtacatgcattggtcagattcacggtttattatgtcatgacttaagttcatggtagtacattacctgccacgcatgcattatattggttagatttgataggcaaatttggttgtcaacaatataaatataaataatgtttttatattatgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataaagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaaaa

SEQ ID NO: 39 - DNA, 75 bp, Glycine max
atgaagagcaccatcttctttgctctctttctcttttgtgccttcaccacctcatacctaccttcagccatcgct

SEQ ID NO: 40 - DNA, 1535 bp, Glycine max
tataaacaccactttaatttgactcggatacatgcatccataaagactacaaaaggcaaaaagagaaggaaatgagatacgaatatatgtcataagtatatataggtgacaagggcaaattaaataggttggtatttaaatgcaaaatcctatgtttgataaagaatggtatgaaaaacaggcaaagttaattgcaattcaaaggtgaacaaagcatttctttgtctacactaatggcatgtctaagtaaattattagtcttgtatctatatgtccacaagttattaattagtcttatactatcaaaaacaagttaagttgcaaatcaaacatgaacaaagcatttgtgttgtaacctacgaaaaaataccctaacatactgatacgaataatgtggcctaaattgatcgtttaccaaattacggtgctggaaaaaaaaattgctcctttaccaacaaaattaagaactgatacatcttgttttttgtcactgaagataaacacgtgatctttggcaaaacataaaggccaacaaaacaaacttgtctcatccctgaatgattcgaatgccatcgtatgcgtgtcacaaagtggaatacagcaatgaacaaatgctatcctcttgagaaaagtgaatgcagcagcagcagcagactagagtgctacaaatgcttatcctcttgagaaaagtgaatgcagcggcagcagacctgagtgctatatacaattagacacagggtctattaattgaaattgtcttattattaaatatttcgttttatattaattttttaaattttaattaaatttatatatattatatttaagacagatatatttatttgtgattataaatgtgtcactttttcttttagtccatgtattcttctattttttcaatttaactttttatttttatttttaagtcactcttgatcaagaaaacattgttgacataaaactattaacataaaattatgttaacatgtgataacatcatattttactaatataacgtcgcattttaacgtttttttaacaaatatcgactgtaagagtaaaaatgaaatgtttgaaaaggttaattgcatactaactattttttttcctataagtaatcttttttgggatcaattgtatatcattgagatacgatattaaatatgggtaccttttcacaaaacctaacccttgttagtcaaaccacacataagagaggatggatttaaaccagtcagcaccgtaagtatatagtgaagaaggctgataacacactctattattgttagtacgtacgtatttccttttttgtttagtttttgaatttaattaattaaaatatatatgctaacaacattaaattttaaatttacgtctaattatatattgtgatgtataataaattgtcaacctttaaaaattataaaagaaatattaattttgataaacaacttttgaaaagtacccaataatgctagtataaataggggcatgactccccatgcatcacagtgcaatttagctgaagcaaagca

SEQ ID NO: 41 - DNA, 96 bp, Glycine max
atggctacttcaaagttgaaaacccagaatgtggttgtatctctctccctaaccttaaccttggtactggtgctactgaccagcaaggcaaactca

SEQ ID NO: 42 - DNA, 1510 bp, Glycine max
tgggtgggaaaacacttataatatatgattcaagaaaagaaagtaaataaaacaattttattatataaagactattaggataaaaaaaaccttaaaagtgcttggatttggaccagacttgaattttaatttaatgatattataatatgtgaatatatttttgagacaattgtaaatttcagataaaaaaataatgtaattaaaattgtaataactatatcgtatacttaattaattattaaatgtgacaaaaaagatatacatcaaaacttaatgtttcatgactttttttttaatgtgtgtcctaaatagaaattaaaaataaaaattattatatccaaatgaaaaaaacatttaatacgtattatttaagaaataacaatatatttatattttaatatgtattcacatgtaaatttaaaaacaaaaacaaaatttctcttttattgattaattaaaataattttataactacatttattttctattattatcaattttcttctgtttttttatttggcatatatacctagacaagtcaaaaaatgactattctttaataatcaatcattattcttacatattgttcgaactacgagttatgaagtgtcaattgcaccttagtgttttgataggcctccatttgccgctcattaattaatttgataacagccgtaccgatcaattacttatgcttcttccatcgtaattatatgcatgtcggttcttttaatcttggtactctcgaatgccaccacaacactgactagtctcttggatcatgagaaaaagccaaagaacaaaaaagacaacataaagagtatcctttgcaaaaaaatgtctaagttcataaaatacaaacaaaaacgcaatcacacacagtggacccaaaagccatgcacaacaacacgtactcaccaaggtgcaatcgtgctgcccaaaaacattcaccaactcaatccatgatgagcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatact

SEQ ID NO: 43 - DNA, 66 bp, Glycine max
atgatgagagcacggttcccattactgttgctgggacttgttttcctggcttcagtttctgtctca

SEQ ID NO: 44 - DNA, 1454 bp, Glycine max
caactattatcgcatgatgatgtacgttaagtcatcatcatctttaactttatatattgttaaaagtagaaaaaataggtgatgcattataaaataattttataacatcatttaattataaattatttataataaatatttgagtttttatagtaattacctaaacaattatatcaagactaatgcctgattagttgacatgacgaaattaaactcataaaagtaaagatgtttatgtggaaaactcttatacaattgagcggacttttttccatggtagttcagttttcttctattcaatttatttttttggtttccgctcagaataagaataatttgataaattcatttttaggcaattaagaatatttatttgactaactttttaattgaaataaatttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatact

SEQ ID NO: 45 - DNA, 72 bp, Glycine max
atgatgagagcgcggttcccattactgttgctgggagttgttttcctggcatcagtttctgtctcatttggc

SEQ ID NO: 46 - DNA, 1263 bp, Glycine max
ccgcacaatacaagtcgccttctcaaacccttcctcacgacattctcggaccttatgacaccgtcaccaaaacaatgatccacgcgatattaggcgcgtgcaaatcactctaatccgaaactagtagacatgggaagcacgagctatacgcgagcgtttcaattgccgccacgaaagcagagaaggccagaaacggaaccacggtaaaatggtaagggtattttcgtaaacagaagaaaagagttgtagctataaataaaccctctaacccacggcgcactattctcttcactccttcgttcactcttcttctcttgcggctagggttttagcgcagcttcttctaggttcgttctcttccgccgctctatggattttaaaccttcgaatcatgtttattccattgaattatgttgcttgcagtttatattttctgaatctgtagttgttgtcttcaatttatcctatgctttatagatcaatcttttgtgtgtgtagtacgtaatttttgttctttttgcttttcgttcaagttgttgggaataatcggggtatcatgttttgatattgtttgttttcttttttgactgcttaataatttttaagttggttttggttttggggttttatgtgcttgttatattcaaatctttggatccagatcttacaaaagttttgggtttaaggatgtttttggctgatgatgaatagatctataaactgttccttttaatcgattcaagcttaggattttactaggcttttgcgaataaatacgtgacagtaagctaattatgtcctttttttgtctcaatcatatctgtctgggtgtgccataatttgtgatatgtctatctggtagaatcttgtgttttatgctttacgatttggtatacctgtttttgaacttgttgtatgatgggtatttagatcaccctatcttttttatgcttctggaagttttatgtaaatgtcgaatatcttaatgttgttgaacttataatgttgtgttgatgtatgtatgatggttttgacaacttttttcactggttctgaaagttttatgtaaattgcaaatatgttaatgttgttgaacttattttttttccttcgatgttgttttgatgtatgtatgatggttttcaccgtagtttctatggctaatatcttaatgttgttgagcttatttttttccttatatgttgtgttgatgtatgtatgatggttttgacaacttttttagtttctttgcagatttaagga

SEQ ID NO: 47 - DNA, 1418 bp, Glycine max
cctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaa

SEQ ID NO: 48 - DNA, 1574 bp, Glycine max
ttacgtgtgctgtgagacattatcatcaattgtgttgtatatgatatatagatatagatatataaatatatagattgagtgatataatatatttaaaatataaattatatatatgttttaatatatttttgcatatatatatatatttgtaaaaactagaagtattttttcatgagataattattatcgagttgaataagtctattatttgtgagagccaaccatatttatatatgtgattaaattttatctttgtgaaattaaaaataataaaaaataccttaaaatcataataatagaaaaacttatatttataatttaccattatacttaaaaaaaattaaataaatattataaatataaatactatcgagtaatggccgcgctagggtttttgagaaaaaatcttcccacgcactcaactgcactgtacggcgtcgttttcacagccgcataatagaagccgcgttccccaacccttcctcacaacattctcggaccctccagcaccgtcacccaaacaaatatccacgcggtagtaggcgcgtgaaacaaactctaatccgaactacgagacgtgagaagcacgcgctttagcgagcgtttcaattgtcgctacgaaagcagagaaggatacaaacggaactagggtaaattagtaagggtaatttcgtaaacagaagaaaagagttgtagctataaataaaccctctaaccctcgtcgcattacttctcttcacacctttgttcactcttcttctcttgcggctagggttttagcgcagcttcttctaggttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcagatttaagga

SEQ ID NO: 49 - DNA, 1574 bp, Artificial Sequence
  variant of GmSM8 promoter
ttacgtgtgctgtgagacattatcatcaattgtgttgtatatgatatatagatatagatatataaatatatagattgagtgatataatatatttaaaatataaattatatatatgttttaatatatttttgcatatatatatatatttgtaaaaactagaagtattttttcatgagataattattatcgagttgaataagtctattatttgtgagagccaaccatatttatatatgtgattaaattttatctttgtgaaattaaaaataataaaaaataccttaaaatcataataatagaaaaacttatatttataatttaccattatacttaaaaaaaattaaataaatattataaatataaatactatcgagtaatggccgcgctagggtttttgagaaaaaatcttcccacgcactcaactgcactgtacggcgtcgttttcacagccgcataatagaagccgcgttccccaacccttcctcacaacattctcggaccctccagcaccgtcacccaaacaaatatccacgcggtagtaggcgcgtgaaacaaactctaatccgaactacgagacgtgagaagcacgcgctttagcgagcgtttcaattgtcgctacgaaagcagagaaggatacaaacggaactagggtaaattagtaagggtaatttcgtaaacagaagaaaagagttgtagctataaataaaccctctaaccctcgtcgcattacttctcttcacacctttgttcactcttcttctcttgcggctagggttttagcgcagcttcttctaggttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgtgtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatttgttatggttttgacaactttttttttttctggatttttgcagatttaagga

SEQ ID NO: 50 - protein, 182 aa, Homo sapiens
MKSFLLVVNALALTLPFLAVEVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPAPATEPTVDSVVTPEAFSESIITSTPETTTVAVTPPTA

SEQ ID NO: 51 - protein, 192 aa, Capra hircus
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 52 - protein, 190 aa, Bos taurus; identical to SEQ ID NO: 5

SEQ ID NO: 53 - protein, 190 aa, Bubalus bubalis
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVTSTVV

SEQ ID NO: 54 - protein, 226 aa, Homo sapiens
MKVLILACLVALALARETIESLSSSEESITEYKQKVEKVKHEDQQQGEDEHQDKIYPSFQPQPLIYPFVEPIPYGFLPQNILPLAQPAVVLPVPQPEIMEVPKAKDTVYTKGRVMPVLKSPTIPFFDPQIPKLTDLENLHLPLPLLQPLMQQVPQPIPQTLALPPQPLWSVPQPKVLPIPQQVVPYPQRAVPVQALLLNQELLLNPTHQIYPVTQPLAPVHNPISV

SEQ ID NO: 55 - protein, 222 aa, Capra hircus
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 56 - protein, 224 aa, Bubalus bubalis
MKVLILACLVALALARELEELNVPGEIVESLSSSEESITHINKKIEKFQSEEQQQMEDELQDKIHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 57 - protein, 185 aa, Homo sapiens
MRLLILTCLVAVALARPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 58 - protein, 214 aa, Capra hircus
MKLLILTCLVAVALARPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 59 - protein, 214 aa, Bos taurus; identical to SEQ ID NO: 11

SEQ ID NO: 60 - protein, 214 aa, Bubalus bubalis
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSGKTTMPLW

SEQ ID NO: 61 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 62 - protein, 222 aa, Bos taurus; identical to SEQ ID NO: 12

SEQ ID NO: 63 - protein, 213 aa, Bubalus bubalis
MKFFIFTCLLAVALAKHTMEHVSSSEESIISQETYKQEKNMAIHPSKENLCSTFCKEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

(19) United States
(12) Patent Application Publication
(10) Pub. No.: US 2020/0123556 A1
(43) Pub. Date: Apr. 23, 2020

(54) MILK PROTEIN PRODUCTION IN TRANSGENIC PLANTS

(71) Applicant: Alpine Roads, Inc., San Francisco, CA (US)

(72) Inventors: Magi El-Richani, San Francisco, CA (US); Shu Li, San Francisco, CA (US)

(73) Assignee: Alpine Roads, Inc., San Francisco, CA (US)

(21) Appl. No.: 16/423,654

(22) Filed: May 28, 2019

Related U.S. Application Data
(63) Continuation of application No. 15/947,596, filed on Apr. 6, 2018, now abandoned.
(60) Provisional application No. 62/483,157, filed on Apr. 7, 2017.
(60) Provisional application No. 62/539,786, filed on Aug. 1, 2017.

(51) Int. Cl. C12N 15/82; C07K 14/76; C12N 9/36; C07K 14/47; C07K 14/79; C12N 9/08; C07K 14/765

(52) U.S. Cl. CPC C12N 15/8257; C07K 14/76; C12N 9/2462; C07K 14/4732; C12N 2800/22; C12N 9/0065; C07K 14/765; C07K 14/4717; C12Y 111/01007; C07K 14/79

(57) ABSTRACT

The disclosure describes a transgenic dicot or monocot plant having bovine milk protein(s) and methods of producing the transgenic dicot or monocot plant containing bovine milk protein(s). These transgenic dicot or monocot plants can express and produce bovine milk protein(s). The methods involve introducing a recombinant DNA construct expressing a bovine milk protein into a dicot or monocot plant, obtaining the dicot or monocot plant containing the bovine milk protein(s) from a recombinant DNA construct, cultivating and harvesting the transgenic dicot or monocot plant, and extracting and purifying the bovine milk protein(s) from transgenic dicot or monocotyledonous plants.

### CROSS-REFERENCE TO RELATED APPLICATIONS

This application a Continuation Application of U.S. patent application Ser. No. 15/947,596, filed on Apr. 6, 2018, which itself claims the benefit of priority to U.S. provisional application No. 62/539,786 filed on Aug. 1, 2017, and U.S. provisional application No. 62/483,157 filed on Apr. 7, 2017, the entire contents of which are hereby incorporated by reference in their entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: ALRO_002_01US_SeqList_ST25.txt, date recorded, Apr. 5, 2018, file size 89.1 kilobytes.

### FIELD

The present disclosure generally relates to production, extraction, and purification of milk proteins from transgenic plants.

### BACKGROUND

Globally, more than 6 billion people around the world consume milk and milk products. Demand for cow milk and dairy products is expected to keep increasing due to increased reliance on these products in developing countries as well as growth in human population which is expected to exceed 9 billion people by 2050.

Relying on animal agriculture to meet growing demand for food is not a sustainable solution. According to the Food & Agriculture Organization of the United Nations, animal agriculture is responsible for 18% of all greenhouse gases, more than the entire transportation sector combined. Dairy cows alone account for 3% of this total.

In addition to impacting the environment, animal agriculture poses a serious risk to human health. A startling 80% of antibiotics used in the United States go towards treating animals, resulting in the development of antibiotic resistant microorganisms also known as superbugs. For years, food companies and farmers have used antibiotics not only to treat sick animals, but also to feed them a steady diet of the drugs to prevent illnesses. In September 2016, the United Nations announced the use of antibiotics in the food system as a crisis on par with Ebola and HIV.

According to the World Dairy Situation Report 2011 published by the International Dairy Federation, cow milk accounted for 83% of global milk production. At present, there is a need of providing bovine milk and/or producing essential high-quality proteins from bovine milk in a more sustainable and humane manner, instead of solely relying on animal farming, in order to produce milk extracts and essential milk protein concentrates or isolates. Also, there is a need for selectively producing the high-quality proteins that are more beneficial than others nutritionally and clinically. Recombinant proteins have been produced and marketed in numerous agricultural, industrial, and pharmaceutical uses. The sustainable production of important recombinant proteins is necessary to provide abundant amounts of the high-quality proteins for commercial applications. These valuable proteins can be efficiently produced in living cells such as bacteria, mammalian, and even plant cells. The subject disclosure described herein provides for a solution to produce essential milk proteins in transgenic plants in a safe, humane and sustainable way.

### SUMMARY OF THE DISCLOSURE

The present disclosure is based, in part, on the observation that transgenic plants having nucleic acid sequences coding for mammalian milk proteins can produce the milk proteins. In some embodiments, the mammalian milk proteins used in the present invention can be from any mammal that produces milk, including but not limited to a mammal selected from the group consisting of bovine, human, goat, sheep, camel, buffalo, water buffalo, dromedary, llama and any combination thereof.

The present disclosure is based, in part, on the observation that transgenic plants having bovine milk proteins can be generated by processes of producing transgenic plants containing bovine milk proteins. Bovine casein and whey proteins that are efficiently expressed from chimeric genes in plants are valuable in terms of producing milk proteins in the plants. Appropriate construction of recombinant constructs/vectors/plasmids having milk protein-coding nucleic acid sequences is critical in order to produce high-quality milk proteins. Codon-optimized nucleic acids can be synthesized based on the genetic and genomic information of a host plant, thus decreasing the risks associated with expressing milk proteins of mammal origin in non-mammal species. Also, the present disclosure involves methods of obtaining, cultivating, and harvesting the transgenic plants by introducing recombinant constructs/vectors/plasmids containing milk protein-coding sequences into the host plants, as well as extracting and purifying the milk proteins expressed in the transgenic plants.

In some embodiments, the present disclosure teaches production, extraction, and purification of bovine milk proteins from transgenic plants. In other embodiments, the present disclosure teaches production, extraction, and purification of milk proteins from the transgenic plants that are genetically engineered. In some embodiments the bovine milk proteins produced and obtained as provided herein can be consumed directly or can be incorporated into any food composition, any feed composition or any beverage in place of or in addition to bovine milk products obtained directly from bovines.

In other embodiments, the present disclosure teaches a transgenic plant comprising a recombinant DNA construct, said construct comprising (i) a promoter, (ii) a nucleic acid sequence encoding a bovine milk protein and/or a functional fragment thereof, which is operably linked to said promoter, and (iii) a termination sequence; wherein the bovine milk protein and/or the functional fragment thereof is expressed in the transgenic plant and/or a part thereof.

In some embodiments, the present disclosure teaches a transgenic plant comprising a recombinant DNA construct, said construct comprising (i) a promoter, (ii) a nucleic acid sequence encoding a bovine milk protein and/or a functional fragment thereof, which is operably linked to said promoter, and (iii) a termination sequence; wherein the bovine milk protein and/or the functional fragment thereof is expressed in the transgenic plant and/or a part thereof, wherein the promoter is selected from the group consisting of a Cauliflower Mosaic Virus (CaMV) 35S promoter, plant constitutive promoters, and plant tissue-specific promoters.

In some embodiments, the present disclosure teaches a transgenic plant comprising a recombinant DNA construct, said construct comprising (i) a promoter, (ii) a nucleic acid sequence encoding a bovine milk protein and/or a functional fragment thereof, which is operably linked to said promoter, and (iii) a termination sequence; wherein the bovine milk protein and/or the functional fragment thereof is expressed in the transgenic plant and/or a part thereof, wherein the promoter is selected from the group consisting of a Cauliflower Mosaic Virus (CaMV) 35S promoter, plant constitutive promoters, and plant tissue-specific promoters; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.

In other embodiments, the present disclosure teaches a transgenic plant comprising a recombinant DNA construct, said construct comprising (i) a promoter, (ii) a nucleic acid sequence encoding a bovine milk protein and/or a functional fragment thereof, which is operably linked to said promoter, and (iii) a termination sequence; wherein the bovine milk protein and/or the functional fragment thereof is expressed in the transgenic plant and/or a part thereof, wherein the promoter is selected from the group consisting of a Cauliflower Mosaic Virus (CaMV) 35S promoter, plant constitutive promoters, and plant tissue-specific promoters; wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase, and wherein the DNA construct contains at least one selectable marker gene.

In further embodiments, the present disclosure teaches a transgenic plant comprising a recombinant DNA construct, said construct comprising (i) a promoter, (ii) a nucleic acid sequence encoding a bovine milk protein and/or a functional fragment thereof, which is operably linked to said promoter, and (iii) a termination sequence; wherein the bovine milk protein and/or the functional fragment thereof is expressed in the transgenic plant and/or a part thereof, wherein the promoter is selected from the group consisting of a Cauliflower Mosaic Virus (CaMV) 35S promoter, plant constitutive promoters, and plant tissue-specific promoters; wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase, wherein the DNA construct contains at least one selectable marker gene, and wherein the termination sequence is a NOS terminator.

In some embodiments, the promoter is a CaMV 35S promoter.

In some embodiments, plant constitutive promoters comprise constitutive promoters derived from soybean, lima bean, Arabidopsis, tobacco, duckweed, rice, maize, barley, sorghum, wheat and/or oat. In other embodiments, plant constitutive promoters comprise soybean constitutive promoters, lima bean constitutive promoters, Arabidopsis constitutive promoters, tobacco constitutive promoters, duckweed constitutive promoters, and rice constitutive promoters. In further embodiments, the promoter is soybean constitutive promoters such as a GmSM8 promoter and a modified GmSM8 promoter including GmSM8-1 promoter.

In some embodiments, plant tissue-specific promoters comprise tissue-specific and/or tissue-preferential promoters derived from soybean, lima bean, Arabidopsis, tobacco, duckweed, rice, maize, barley, sorghum, wheat and/or oat. In other embodiments, plant tissue-specific promoters comprise soybean tissue-specific promoters, lima bean tissue-specific promoters, Arabidopsis tissue-specific promoters, tobacco tissue-specific promoters, duckweed tissue-specific promoters, and rice tissue-specific promoters. In further embodiments, the promoter is soybean tissue-specific promoters such as AR-Pro1, AR-Pro2, AR-Pro3, AR-Pro4, AR-Pro5, AR-Pro6, AR-Pro7, AR-Pro8, and AR-Pro9 promoters.

In some embodiments, the present disclosure teaches that a transgenic plant is a transgenic monocotyledonous (monocot) plant. In some embodiments, the present disclosure teaches that a transgenic monocot plant is selected from the group consisting of turf grass, maize (corn), rice, oat, wheat, barley, sorghum, orchid, iris, lily, onion, palm, and duckweed. In other embodiments, the present disclosure teaches that a transgenic plant is a monocot plant, such as maize, oat, barley, wheat, rice and duckweed.

In some embodiments, the present disclosure teaches that a transgenic plant is a non-vascular plant such as moss, liverwort, hornwort and algae. In other embodiments, the present disclosure teaches that a transgenic plant is a vascular plant reproducing from spores such as fern.

In some embodiments, the present disclosure teaches that a transgenic plant is a transgenic dicotyledonous (dicot) plant. In some embodiments, the present disclosure teaches a transgenic dicot plant selected from the group consisting of Arabidopsis, tobacco, tomato, potato, sweet potato, cassava, legumes including alfalfa, lima bean, pea, chick pea, soybean, carrot, strawberry, lettuce, oak, maple, walnut, rose, mint, squash, daisy, and cactus. In other embodiments, the present disclosure teaches a transgenic dicot plant selected from the group consisting of soybean, lima bean, Arabidopsis, and tobacco.

The present disclosure provides identification and use of nucleic acid sequences encoding a bovine milk protein and/or a functional fragment thereof for producing the bovine milk protein in plants. Importantly, the bovine milk protein can be obtained from the transgenic plants that are produced and maintained using conventional plant breeding methods, which include any of various biotechnological methods for verifying that the desired nucleic acid sequences encoding the bovine milk protein and/or the functional fragments and variation thereof are present and/or expressed in the transgenic plants. Further, the transgenic plants and progenies thereof can be produced by resulting crosses.

In some embodiments, the present disclosure provides nucleic acid sequences encoding a bovine milk protein and functional fragments and variations thereof, and allows for the design of gene-specific primers and probes for the nucleic acid sequences encoding the bovine milk proteins, and/or the functional fragments and variations thereof. The present disclosure also provides chimeric genes or heterologous DNA, recombinant DNA, constructs, vectors, plasmids, plant cells, plant tissues, plant parts, plant tissue cultures and/or whole plants comprising such nucleic acid sequences.

In some embodiments, a nucleic acid sequence and/or a functional fragment thereof is a coding sequence for the bovine milk protein selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase. In other embodiments, a nucleic acid sequence and/or a functional fragment thereof is a codon-optimized sequence selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase. In further embodiments, a nucleic acid sequence and/or a functional fragment thereof is a codon-optimized sequence selected from the group consisting of α-S1 casein, α-S2 casein, β-casein, κ-casein, α-lactalbumin, β-lactoglobulin and lysozyme. In such embodiments a nucleic acid sequence and/or a functional fragment thereof is a codon-optimized sequence selected from the group consisting of β-casein and κ-casein. In such embodiments a nucleic acid sequence and/or a functional fragment thereof is a codon-optimized sequence selected from the group consisting of α-S1 casein and α-S2 casein. In such embodiments a nucleic acid sequence and/or a functional fragment thereof is a codon-optimized sequence selected from the group consisting of α-lactalbumin, β-lactoglobulin and lysozyme.

In another embodiment, a protein-coding sequence and/or a functional fragment thereof is a coding sequence for the bovine milk protein selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase. In some embodiments, a protein-coding sequence and/or a functional fragment thereof is a codon-optimized sequence selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase. In further embodiments, a protein-coding sequence and/or a functional fragment thereof is a codon-optimized sequence selected from the group consisting of α-S1 casein, α-S2 casein, β-casein, κ-casein, α-lactalbumin, β-lactoglobulin and lysozyme. In such embodiments, a protein-coding sequence and/or a functional fragment thereof is a codon-optimized sequence selected from the group consisting of β-casein and κ-casein. In such embodiments a nucleic acid sequence and/or a functional fragment thereof is a codon-optimized sequence selected from the group consisting of α-S1 casein and α-S2 casein. In such embodiments a nucleic acid sequence and/or a functional fragment thereof is a codon-optimized sequence selected from the group consisting of α-lactalbumin, β-lactoglobulin and lysozyme.

The present disclosure further provides a codon-optimized version of the bovine milk protein-coding genes that is synthesized for expression in plants. In some embodiments, the codon-optimized version of the bovine milk protein-coding genes is synthesized for expression in plants selected from the group consisting of soybean, lima bean, Arabidopsis, tobacco, rice and duckweed.

In some embodiments, the present disclosure teaches that the bovine milk protein is α-S1 casein, α-S2 casein, β-casein, κ-casein, α-lactalbumin, β-lactoglobulin or lysozyme.

In some embodiments, the present disclosure teaches that a nucleic acid sequence encoding β-casein is codon-optimized. In some embodiments, a codon-optimized version of β-casein is synthesized for expression in soybean. In some embodiments, a codon-optimized version of β-casein is synthesized for expression in lima bean. In some embodiments, a codon-optimized version of β-casein is synthesized for expression in Arabidopsis. In some embodiments, a codon-optimized version of β-casein is synthesized for expression in tobacco. In some embodiments, a codon-optimized version of β-casein is synthesized for expression in rice. In some embodiments, a codon-optimized version of β-casein is synthesized for expression in duckweed.

In further embodiments, the present disclosure teaches that a nucleic acid sequence encoding κ-casein is codon-optimized. In some embodiments, a codon-optimized version of κ-casein is synthesized for expression in soybean. In some embodiments, a codon-optimized version of κ-casein is synthesized for expression in lima bean. In some embodiments, a codon-optimized version of κ-casein is synthesized for expression in Arabidopsis. In some embodiments, a codon-optimized version of κ-casein is synthesized for expression in tobacco. In some embodiments, a codon-optimized version of κ-casein is synthesized for expression in rice. In some embodiments, a codon-optimized version of κ-casein is synthesized for expression in duckweed.

In further embodiments, the present disclosure teaches that a nucleic acid sequence encoding α-S1 casein is codon-optimized. In some embodiments, a codon-optimized version of α-S1 casein is synthesized for expression in soybean. In some embodiments, a codon-optimized version of α-S1 is synthesized for expression in lima bean. In some embodiments, a codon-optimized version of α-S1 casein is synthesized for expression in Arabidopsis. In some embodiments, a codon-optimized version of α-S1 casein is synthesized for expression in tobacco. In some embodiments, a codon-optimized version of α-S1 casein is synthesized for expression in rice. In some embodiments, a codon-optimized version of α-S1 casein is synthesized for expression in duckweed.

In further embodiments, the present disclosure teaches that a nucleic acid sequence encoding α-S2 casein is codon-optimized. In some embodiments, a codon-optimized version of α-S2 casein is synthesized for expression in soybean. In some embodiments, a codon-optimized version of α-S2 casein is synthesized for expression in lima bean. In some embodiments, a codon-optimized version of α-S2 casein is synthesized for expression in Arabidopsis. In some embodiments, a codon-optimized version of α-S2 casein is synthesized for expression in tobacco. In some embodiments, a codon-optimized version of α-Ω casein is synthesized for expression in rice. In some embodiments, a codon-optimized version of α-Ω casein is synthesized for expression in duckweed.

In further embodiments, the present disclosure teaches that nucleic acid sequences encoding α-lactalbumin is codon-optimized. In some embodiments, a codon-optimized version of α-lactalbumin is synthesized for expression in soybean. In some embodiments, a codon-optimized version of α-lactalbumin is synthesized for expression in lima bean. In some embodiments, a codon-optimized version of α-lactalbumin is synthesized for expression in Arabidopsis. In some embodiments, a codon-optimized version of α-lactalbumin is synthesized for expression in tobacco. In some embodiments, a codon-optimized version of α-lactalbumin is synthesized for expression in rice. In some embodiments, a codon-optimized version of α-lactalbumin is synthesized for expression in duckweed.

In further embodiments, the present disclosure teaches that a nucleic acid sequence encoding β-lactoglobulin is codon-optimized. In some embodiments, a codon-optimized version of β-lactoglobulin is synthesized for expression in soybean. In some embodiments, a codon-optimized version of β-lactoglobulin is synthesized for expression in lima bean. In some embodiments, a codon-optimized version of β-lactoglobulin is synthesized for expression in Arabidopsis. In some embodiments, a codon-optimized version of β-lactoglobulin is synthesized for expression in tobacco. In some embodiments, a codon-optimized version of β-lactoglobulin is synthesized for expression in rice. In some embodiments, a codon-optimized version of β-lactoglobulin is synthesized for expression in duckweed.

In further embodiments, the present disclosure teaches that a nucleic acid sequence encoding lysozyme is codon-optimized. In some embodiments, a codon-optimized version of lysozyme is synthesized for expression in soybean. In some embodiments, a codon-optimized version of lysozyme is synthesized for expression in lima bean. In some embodiments, a codon-optimized version of lysozyme is synthesized for expression in Arabidopsis. In some embodiments, a codon-optimized version of lysozyme is synthesized for expression in tobacco. In some embodiments, a codon-optimized version of lysozyme is synthesized for expression in rice. In some embodiments, a codon-optimized version of lysozyme is synthesized for expression in duckweed.

The present disclosure provides a chimeric gene comprising the nucleic acid sequence of any one of the nucleic acid sequences described herein operably linked to suitable regulatory sequences that include 5′ upstream and 3′ downstream. The present disclosure also provides recombinant DNA constructs comprising the chimeric genes as described herein.

Other aspects of the present disclosure provide transgenic plants comprising in their genome chimeric genes as described herein. In some embodiments, transgenic plants are derived from a soybean variety, wherein a chimeric gene comprises a nucleic acid sequence encoding a bovine milk protein. In some embodiments, transgenic plants are derived from a soybean variety, and wherein a chimeric gene comprises a nucleic acid sequence encoding β-casein. In other embodiments, transgenic plants are derived from a soybean variety, wherein a chimeric gene comprises a nucleic acid sequence encoding κ-casein. In some embodiments, transgenic plants are derived from a soybean variety, and wherein a chimeric gene comprises a nucleic acid sequence encoding α-S1 casein. In other embodiments, transgenic plants are derived from a soybean variety, wherein a chimeric gene comprises a nucleic acid sequence encoding α-S2 casein. In some embodiments, transgenic plants are derived from a soybean variety, wherein a chimeric gene comprises a nucleic acid sequence encoding α-lactalbumin. In other embodiments, transgenic plants are derived from a soybean variety, wherein a chimeric gene comprises a nucleic acid sequence encoding β-lactoglobulin. In other embodiments, transgenic plants are derived from a soybean variety, wherein a chimeric gene comprises a nucleic acid sequence encoding lysozyme.

In some embodiments, transgenic plants are derived from a lima bean variety, wherein a chimeric gene comprises a nucleic acid sequence encoding a bovine milk protein. In other embodiments, transgenic plants are derived from a lima bean variety, wherein a chimeric gene comprises a nucleic acid sequence encoding β-casein. In further embodiments, transgenic plants are derived from a lima bean variety, wherein a chimeric gene comprises a nucleic acid sequence encoding κ-casein. In some embodiments, transgenic plants are derived from a lima bean variety, wherein a chimeric gene comprises a nucleic acid sequence encoding α-S1 casein. In other embodiments, transgenic plants are derived from a lima bean variety, wherein a chimeric gene comprises a nucleic acid sequence encoding α-S2 casein. In further embodiments, transgenic plants are derived from a lima bean variety, wherein a chimeric gene comprises a nucleic acid sequence encoding α-lactalbumin. In some embodiments, transgenic plants are derived from a lima bean variety, wherein a chimeric gene comprises a nucleic acid sequence encoding β-lactoglobulin. In other embodiments, transgenic plants are derived from a lima bean variety, wherein a chimeric gene comprises a nucleic acid sequence encoding lysozyme.

In some embodiments, transgenic plants are derived from an Arabidopsis variety, wherein a chimeric gene comprises a nucleic acid sequence encoding a bovine milk protein. In other embodiments, transgenic plants are derived from an Arabidopsis variety, wherein a chimeric gene comprises a nucleic acid sequence encoding β-casein. In further embodiments, transgenic plants are derived from an Arabidopsis variety, wherein a chimeric gene comprises a nucleic acid sequence encoding κ-casein. In some embodiments, transgenic plants are derived from an Arabidopsis variety, wherein a chimeric gene comprises a nucleic acid sequence encoding α-S1 casein. In other embodiments, transgenic plants are derived from an Arabidopsis variety, wherein a chimeric gene comprises a nucleic acid sequence encoding α-S2 casein. In further embodiments, transgenic plants are derived from an Arabidopsis variety, wherein a chimeric gene comprises a nucleic acid sequence encoding α-lactalbumin. In some embodiments, transgenic plants are derived from an Arabidopsis variety, wherein a chimeric gene comprises a nucleic acid sequence encoding β-lactoglobulin. In other embodiments, transgenic plants are derived from an Arabidopsis variety, wherein a chimeric gene comprises a nucleic acid sequence encoding lysozyme.

In some embodiments, transgenic plants are derived from a tobacco variety, wherein a chimeric gene comprises a nucleic acid sequence encoding a bovine milk protein. In other embodiments, transgenic plants are derived from a tobacco variety, wherein a chimeric gene comprises a nucleic acid sequence encoding β-casein. In further embodiments, transgenic plants are derived from a tobacco variety, wherein a chimeric gene comprises a nucleic acid sequence encoding κ-casein. In some embodiments, transgenic plants are derived from a tobacco variety, wherein a chimeric gene comprises a nucleic acid sequence encoding α-S1 casein. In other embodiments, transgenic plants are derived from a tobacco variety, wherein a chimeric gene comprises a nucleic acid sequence encoding α-S2 casein. In further embodiments, transgenic plants are derived from a tobacco variety, wherein a chimeric gene comprises a nucleic acid sequence encoding α-lactalbumin. In some embodiments, transgenic plants are derived from a tobacco variety, wherein a chimeric gene comprises a nucleic acid sequence encoding β-lactoglobulin. In other embodiments, transgenic plants are derived from a tobacco variety, wherein a chimeric gene comprises a nucleic acid sequence encoding lysozyme.

In some embodiments, transgenic plants are derived from a rice variety, wherein a chimeric gene comprises a nucleic acid sequence encoding a bovine milk protein. In other embodiments, transgenic plants are derived from a rice variety, wherein a chimeric gene comprises a nucleic acid sequence encoding β-casein. In further embodiments, transgenic plants are derived from a rice variety, wherein a chimeric gene comprises a nucleic acid sequence encoding κ-casein. In some embodiments, transgenic plants are derived from a rice variety, wherein a chimeric gene comprises a nucleic acid sequence encoding α-S1 casein. In other embodiments, transgenic plants are derived from a rice variety, wherein a chimeric gene comprises a nucleic acid sequence encoding α-S2 casein. In further embodiments, transgenic plants are derived from a rice variety, wherein a chimeric gene comprises a nucleic acid sequence encoding α-lactalbumin. In some embodiments, transgenic plants are derived from a rice variety, wherein a chimeric gene comprises a nucleic acid sequence encoding β-lactoglobulin. In other embodiments, transgenic plants are derived from a rice variety, wherein a chimeric gene comprises a nucleic acid sequence encoding lysozyme.

In some embodiments, transgenic plants are derived from a duckweed variety, wherein a chimeric gene comprises a nucleic acid sequence encoding a bovine milk protein. In other embodiments, transgenic plants are derived from a duckweed variety, wherein a chimeric gene comprises a nucleic acid sequence encoding β-casein. In further embodiments, transgenic plants are derived from a duckweed variety, wherein a chimeric gene comprises a nucleic acid sequence encoding κ-casein. In some embodiments, transgenic plants are derived from a duckweed variety, wherein a chimeric gene comprises a nucleic acid sequence encoding α-S1 casein. In other embodiments, transgenic plants are derived from a duckweed variety, wherein a chimeric gene comprises a nucleic acid sequence encoding α-S2 casein. In further embodiments, transgenic plants are derived from a duckweed variety, wherein a chimeric gene comprises a nucleic acid sequence encoding α-lactalbumin. In some embodiments, transgenic plants are derived from a duckweed variety, wherein a chimeric gene comprises a nucleic acid sequence encoding β-lactoglobulin. In other embodiments, transgenic plants are derived from a duckweed variety, wherein a chimeric gene comprises a nucleic acid sequence encoding lysozyme.

In some embodiments, the present disclosure provides plant seeds obtained from the transgenic plants described herein, wherein the transgenic plants producing such seeds comprise in their genome one or more genes as described herein, one or more genes with mutations as described herein, chimeric genes as described herein, or transgenes as described herein.

In some embodiments, the present disclosure provides immature, mature, and/or somatic embryos obtained from the transgenic plants described herein, wherein the transgenic plants producing such immature, mature, and/or somatic embryos comprise in their genome one or more genes as described herein, one or more genes with mutations as described herein, chimeric genes as described herein, or transgenes as described herein.

In other embodiments, the present disclosure further provides amino acid sequences (e.g., a peptide, polypeptide and protein) comprising an amino acid sequence of the bovine milk proteins selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.

In some embodiments, the present disclosure provides nucleic acid sequences encoding κ-casein protein and/or the functional fragment thereof, having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% sequence or 100% identity to SEQ ID No:5. In some embodiments, the present disclosure provides the nucleic acid sequences encoding β-casein and/or the functional fragment thereof, having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID No:7. In some embodiments, the present disclosure provides nucleic acid sequences encoding α-S1 casein and/or the functional fragment thereof, having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID No:11. In some embodiments, the present disclosure provides nucleic acid sequences encoding α-S2 casein and/or the functional fragment thereof, having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID No:12. In some embodiments, the present disclosure provides nucleic acid sequences encoding α-lactalbumin and/or the functional fragment, having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID No:22. In some embodiments, the present disclosure provides nucleic acid sequences encoding β-lactoglobulin and/or the functional fragment thereof, having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID No:23. In some embodiments, the present disclosure provides nucleic acid sequences encoding lysozyme and/or the functional fragment, having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% sequence or 100% identity to SEQ ID No:24.

As aforementioned, the present disclosure provides transgenic plants having recombinant DNA constructs are able to produce bovine milk proteins by expressing nucleic acid sequences encoding the bovine milk proteins. Thus, the present disclosure provides detailed guidance for methods of producing transgenic plants comprising the recombinant DNA constructs. The disclosure also provides methods of producing the bovine milk proteins from the transgenic plants.

To illustrate the various aspects of the disclosure, several representative embodiments are set forth herein.

In some embodiments, the present disclosure teaches methods of producing a transgenic plant, said methods comprising the steps of: (a) introducing at least one expression cassette capable of expressing a bovine milk protein into a plant, a part thereof, or a cell thereof; (b) obtaining the transgenic plant, the part thereof, or the cell thereof, which stably expresses the bovine milk protein; (c) cultivating the transgenic plant, the part thereof, or the cell thereof; and (d) harvesting the transgenic plant, the part thereof, or the cell thereof. In such embodiments, the transgenic plant is a dicot plant selected from the group consisting of Arabidopsis, tobacco, tomato, potato, sweet potato, cassava, legumes including alfalfa, lima bean, pea, chick pea, soybean, carrot, strawberry, lettuce, oak, maple, walnut, rose, mint, squash, daisy, and cactus. In yet some embodiments, the transgenic dicot plant is selected from the group consisting of soybean, lima bean, Arabidopsis, and tobacco. In such embodiments, the transgenic plant is a monocot plant selected from the group consisting of turf grass, maize (corn), rice, oat, wheat, barley, sorghum, orchid, iris, lily, onion, palm, and duckweed. In yet some embodiments, the transgenic plant is a monocot plant, such as, e.g., maize, oat, barley, wheat, rice and duckweed. In other embodiments, the transgenic plant is a non-vascular plant such as moss. In other embodiments, the transgenic plant is a vascular plant reproducing from spores such as fern.

In some embodiments, the present disclosure teaches that methods of introducing at least one expression cassette capable of expressing a bovine milk protein into a plant, a part thereof, or a cell thereof comprises Agrobacterium-mediated transformation, particle bombardment-medicated transformation, electroporation, and microinjection.

In some embodiments, the present disclosure teaches methods of producing a bovine milk protein from a transgenic plant, said methods comprising the steps of: (a) extracting the bovine milk protein from the transgenic plant, the part thereof, or the cell thereof; and (b) purifying the bovine milk protein from the transgenic plant, the part thereof, or the cell thereof. In other embodiments, the transgenic plant is a dicot plant selected from the group consisting of Arabidopsis, tobacco, tomato, potato, sweet potato, cassava, legumes including alfalfa, lima bean, pea, chick pea, soybean, carrot, strawberry, lettuce, oak, maple, walnut, rose, mint, squash, daisy, and cactus. In yet some embodiments, the transgenic dicot plant is selected from the group consisting of soybean, lima bean, Arabidopsis, and tobacco. In yet some embodiments, the transgenic plant is a monocot plant selected from the group consisting of turf grass, maize (corn), rice, oat, wheat, barley, sorghum, orchid, iris, lily, onion, palm, and duckweed. In yet some embodiments, the transgenic plant is a monocot plant, such as, e.g., maize, oat, barley, wheat, rice and duckweed. In other embodiments, the transgenic plant is a non-vascular plant such as moss. In other embodiments, the transgenic plant is a vascular plant reproducing from spores such as fern.

In some embodiments, the present disclosure teaches methods of producing hybrid seed, said method comprising: crossing a transgenic dicot or monocot plant expressing bovine milk protein(s) with another dicot or monocot plant, and harvesting the resultant seed.

In other embodiments, the present disclosure teaches a hybrid plant grown from a hybrid seed produced by the method of producing such seed, wherein the hybrid plant comprises the recombinant DNA construct for expressing milk proteins from the transgenic dicot or monocot plant.

In further embodiments, the present disclosure teaches methods of producing dicot or monocot plants comprising the recombinant DNA construct for expressing milk proteins, said method comprising: (i) making a cross between a first transgenic dicot or monocot plant with a second dicot or monocot plant to produce an F1 plant; (ii) backcrossing the F1 plant to the second plant; and (iii) repeating the backcrossing step one or more times to generate a near isogenic or isogenic line, wherein the recombinant construct with the nucleic acid encoding a bovine milk protein and/or a functional fragment thereof is integrated into the genome of the second plant and the near isogenic or isogenic line derived from the second plant; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.

### BRIEF DESCRIPTION OF THE FIGURES

FIG. 1 depicts a pCAMBIA1305.1 expression vector with DNA insert (e.g. OKC1 transgene). The vector backbone region is 11,847 bp long. The DNA insert region consists of a DNA sequence that is optimized based the codon usage database of soybean, lima bean, Arabidopsis, tobacco, rice, or duckweed.

FIG. 2A-2B illustrates schematics of transgene constructs for expression of milk proteins including casein proteins and whey proteins in plant tissues. Each transgene (marked with *) is driven by a constitutive CaMV 35S promoter and fused with GUSPlus™ and 6×His-tag. FIG. 2A illustrates four constructs with four distinct types of transgenes encoding casein proteins; i) OKC1 (Optimized Kappa Casein version 1), ii) OKC1-T (Optimized Kappa Casein Truncated version 1), iii) OBC1 (Optimized Beta Casein version 1), and iv) OBC1-T (Optimized Beta Casein Truncated version 1). FIG. 2B illustrates three constructs with three distinct types of transgenes encoding whey proteins; i) OLA1 (Optimized Alpha Lactalbumin version 1), ii) OLG1 (Optimized Beta Lactoglobulin 1), and iii) OLY1 (Optimized Lysozyme C version 1). Transgene sequences are optimized based on the soybean codon usage database.

FIG. 3A illustrates schematics of transgene constructs for expression of milk proteins in plant tissues. Individual transgene (marked with *) is driven by a constitutive CaMV 35S promoter and fused with GFP and 6×His-tag. Six constructs were generated with six distinct types of transgenes encoding casein proteins; i) OKC1 (Optimized Kappa Casein version 1), ii) OKC1-T (Optimized Kappa Casein Truncated version 1), iii) OBC1 (Optimized Beta Casein version 1), iv) OBC1-T (Optimized Beta Casein Truncated version 1), v) OS1C1 (Optimized alpha S1 Casein version 1), vi) OS2C1 (Optimized alpha S2 Casein version 1). Transgene sequences are optimized based the soybean codon usage database.

FIG. 3B illustrates schematics of transgene constructs for expression of milk proteins in plant tissues. Individual transgene is driven by a soybean constitutive GmSM8-1 promoter and fused with GFP and 6×His-tag. Eight constructs are presented with eight distinct types of transgenes encoding milk proteins; i) OKC1 (Optimized Kappa Casein version 1), ii) OKC1-T (Optimized Kappa Casein Truncated version 1), iii) OBC1 (Optimized Beta Casein version 1), iv) OBC1-T (Optimized Beta Casein Truncated version 1), v) OS1C1 (Optimized alpha S1 Casein version 1), vii) OS2C1 (Optimized alpha S2 Casein version 1), and. vi) OS1C1-T (Optimized alpha S1 Casein Truncated version 1), viii) OS2C1-T (Optimized alpha S2 Casein Truncated version 1). Transgene sequences are optimized based the soybean codon usage database. For constructs with truncated milk protein-coding transgenes, signal peptide-coding DNA sequence encoding AR-Pro3 signal peptide is inserted between the promoter and the truncated transgene.

FIG. 3C illustrates schematics of transgene constructs for expression of milk proteins in plant tissues. Individual transgene is driven by a soybean tissue-specific AR-Pro3 promoter and fused with GFP and 6×His-tag. Eight constructs are presented with eight distinct types of transgenes encoding milk proteins; i) OKC1 (Optimized Kappa Casein version 1), ii) OKC1-T (Optimized Kappa Casein Truncated version 1), iii) OBC1 (Optimized Beta Casein version 1), iv) OBC1-T (Optimized Beta Casein Truncated version 1), v) OS1C1 (Optimized alpha S1 Casein version 1), vi) OS1C1-T (Optimized alpha S1 Casein Truncated version 1), vii) OS2C1 (Optimized alpha S2 Casein version 1), and viii) OS2C1-T (Optimized alpha S2 Casein Truncated version 1). Transgene sequences are optimized based on the soybean codon usage database. For constructs with truncated milk protein-coding transgenes, a nucleic acid sequence encoding AR-Pro3 signal peptide is inserted between the promoter and the truncated transgene of interest.

FIG. 4A-4E is a representative GUS staining showing transient expression of κ-casein protein in tobacco leaves using a syringe infiltration method. FIGS. 4A and 4B show OKC1 expression in tobacco leaves and FIGS. 4C and 4D show OKC1-T expression in tobacco leaves, compared to a wild-type (WT) control shown in FIG. 4E.

FIG. 5A-5B is a representative GUS staining showing transient expression of β-casein protein in tobacco leaves using a syringe infiltration method. FIG. 5A shows OBC1 expression in tobacco leaves and FIG. 5B shows OBC1-T expression in tobacco leaves, compared to wild-type (WT) tobacco leaves shown on the left column as a control.

FIG. 6A-6B is a representative GUS staining showing transient expression of whey proteins β-lactoglobulin and α-lactalbumin) in tobacco leaves using a syringe infiltration method. FIG. 6A shows OLG1 expression in tobacco leaves and FIG. 6B shows OLA1 expression in tobacco leaves, compared to a wild-type (WT) tobacco leave shown on the top row as a control.

FIG. 7A-7B is a representative GUS staining showing transient expression of κ-casein protein in soybean leaves through sonication and vacuum infiltration method. FIGS. 7A and 7B show OKC1 and OKC1-T expression in soybean leaves, respectively. Wild-type (WT) soybean leaves are shown on the left column as a control.

FIG. 8A-8B depicts a GUS staining to test transient expression of β-casein protein in soybean leaves through sonication and vacuum infiltration method. FIGS. 8A and 8B show OBC1 and OBC1-T expression in soybean leaves, respectively. Wild-type (WT) soybean leaves are shown on the top row as a control.

FIG. 9 depicts growth of shoots regenerated from tobacco leaf pieces transformed with recombinant constructs for stable expression of κ-casein protein.

FIG. 10A-10B is a representative GUS staining showing stable expression of κ-casein protein in transgenic tobacco leaves after Agrobacterium-mediated transformation of leaf pieces and subsequent regeneration presented in FIG. 9. FIGS. 10A and 10B show OKC1 and OKC1-T expression in stable transgenic tobacco leaves, respectively. A wild-type (WT) tobacco leave is shown on the top row as a control.

FIG. 11 is a result of anti-His western blot analysis showing expression of recombinant truncated κ-casein protein. The OKC1-T:GUSplus:6×His chimeric gene is under the control of the 35S promoter in stable transgenic tobacco leaf tissues. A primary antibody against the poly-histidine epitope was used for the western blot analysis. Lysate was loaded with 50 ug of protein into each sample well. Protein lysates were extracted from stable transgenic tobacco plants, labeled as OKC1-T:GUSplus 009, OKC1-T:GUSplus 010, OKC1-T:GUSplus 011. Purified Bovine Kappa Casein and protein lysate extracted from wild-type (WT) tobacco leave tissues were used as a negative control, respectively.

FIG. 12 shows identification of GUS-fused κ-casein protein on a gel for mass spectrometry analysis. The fusion proteins were extracted from stable transgenic tobacco leaf tissues used in FIG. 11. Protein lysate of wild type (WT) tobacco leave tissues was used as a negative control.

FIG. 13A-13C is a result of Mass Spectrometry analysis showing peptide sequences matched to GUS::6×His protein sequence. Peptide sequences were identified from the truncated κ-casein protein using Mass Spectrometry analysis. FIG. 13A shows that twelve peptide sequences (see underlined), identified from about 90 kDa of the truncated κ-casein protein, match to GUS::6×His protein sequence, and the coverage of protein is 26.7%, while FIG. 13B shows that two peptides (see text underlined) identified from about 15 kDa of the truncated κ-casein protein are found in GUS::6×His protein sequence, with the coverage of protein sequence of 4.2%. FIG. 13C shows that two peptides (see text underlined) identified from Mass Spectrometry analysis matched to α-S1-casein protein with the coverage of 10.3%. Lima bean tissue transiently expressing casein (OS1C1:GFP:6×His) was processed following the procedure as described above, and gel band at about 50 kDa was sent for Mass Spectrometry analysis.

FIG. 14A is a representative GUS staining showing stable expression of the truncated κ-casein protein in rice leaves. FIG. 14A shows OKC1-T expression in stable transgenic rice leaves. A wild-type (WT) tobacco leave is shown on the top row as a control.

FIG. 15 is a result of anti-His western blot analysis showing expression of the truncated κ-casein protein (OKC1-T:GUSplus:6×His under the control of the CaMV 35S promoter) in stable transgenic rice leaf tissues. A primary antibody against the poly-histidine epitope was used for the western blot analysis. Lysate was loaded with 50 ug of protein into each sample well. Protein lysates were extracted from stable transgenic rice plants, OKC1-T:GUSplus 002, OKC1-T:GUSplus 003, and OKC1-T:GUSplus 004. Purified Bovine Kappa Casein (Sigma Aldrich) was used as a negative control.

FIG. 16A is a result of anti-His western blot analysis showing expression of recombinant milk proteins including truncated κ-casein protein and full-length κ-casein protein, (sig:OKC1-T:GFP:6×His and OKC:GFP:6×His under the control of the constitutive GmSM8-1 promoter) in stable transgenic tobacco leaf tissues. A primary antibody against the poly-histidine epitope was used for the western blot analysis. Lysate was loaded with 50 ug of protein into each sample well. Protein lysates were extracted from stable transgenic tobacco plants, as follows: (1) sig:OKC1-T:GFP 003, sig:OKC1-T:GFP 005, sig:OKC1-T:GFP 007, and sig:OKC1-T:GFP 008, (2) OKC1:GFP 003, OKC1:GFP 007, and OKC1-T:GFP 009. Protein lysate extracted from wild type tobacco leave tissues was used as a negative control.

FIG. 16B is a result of anti-His western blot analysis showing expression of recombinant α-S1 casein protein (OS1C1-GFP:6×His and 052C1-GFP:6×His under the control of the constitutive GmSM8-1 promoter) in stable transgenic tobacco leaf tissues. A primary antibody against the poly-histidine epitope was used for the western blot analysis. Lysate was loaded with 50 ug of protein into each sample well. Protein lysates were extracted from stable transgenic tobacco plants, as follows: (1) OS1C1:GFP 001 and OS1C1:GFP 002; (2) OS1C1:GFP 003 and OS2C1:GFP 004. Protein lysate extracted from wild type tobacco leaf tissues was used as a negative control.

FIG. 17 is a result of anti-His western blot analysis showing expression of recombinant milk proteins including α-S1 casein protein, α-S2 casein protein, full-length β-casein and truncated β-casein (OS1C1:GFP:6×His, 052C1:GFP:6×His, OBC1:GFP:6×His, and OBC1-T:GFP:6×His under the control of the constitutive CaMV 35S promoter), which were purified from immature embryogenic soy bean callus using a Ni-NTA column. A primary antibody against the poly-histidine epitope was used for the western blot analysis. Lysate was loaded with 50 ug of protein into each sample well. Protein lysate purified from wild-type embryogenic soy callus was used as a negative control.

FIG. 18 is a result of anti-His western blot analysis showing expression of recombinant milk proteins including β-casein and κ-casein (OBC1:GFP:6×His, and OKC1:GFP:6×His under the control of the constitutive GmSM8-1 promoter), which were purified from immature embryogenic lima bean callus using a Ni-NTA column. A primary antibody against the poly-histidine epitope was used for the western blot analysis. Lysate was loaded with 50 ug of protein into each sample well. Protein lysates were purified from immature embryogenic lima bean callus, as follows: (1) OBC1:GFP:6×His #8-1 and OBC1:GFP:6×His #8-2; (2) OKC1:GFP:6×His #7-1, OKC1:GFP:6×His #7-2, OKC1:GFP:6×His #7mu-1 and OKC1:GFP:6×His #7mu-2. Purified Bovine Kappa Casein was used as a negative control.

FIG. 19 illustrates a representative GFP signal from immature embryogenic lima bean callus tissue transiently expressing OKC1:GFP:6×His under the control of the GmSM8-1 promoter, which is labeled as Construct 7mu. GFP expression is displayed with bright and/or white color of dots, spots or stains.

FIG. 20 illustrates milky eluant resulting from purification of recombinant milk proteins including κ-casein and β-casein (OKC1:GFP:6×His and OBC1:GFP:6×His under the control of the constitutive GmSM8-1 promoter) purified from Lima and Soy bean embryogenic callus tissues.

FIG. 21A is a representative GFP florescence expression showing early stage event carrying construct of recombinant milk protein (OKC1:GFP:6×His and OBC1:GFP:6×His under the control of the constitutive GmSM8-1 promoter) in embryogenic soybean callus. FIG. 21B shows a bright field image of the background under white light as a control.

FIG. 22A shows results of the multiple sequence alignment analysis of human κ-casein protein (GenBank P07498; SEQ ID NO: 50), goat (Capra hircus) κ-casein protein (GenBank P02670; SEQ ID NO: 51), bovine (Bos taurus) κ-casein protein (P02668; SEQ ID NO: 52), and water buffalo (Bubalus bubalis) κ-casein protein (GenBank P11840; SEQ ID NO: 53). FIG. 22B shows results of the percent identity matrix based on the multiple sequence alignment analysis.

FIG. 23A shows results of the multiple sequence alignment analysis of human β-casein protein (GenBank P05814; SEQ ID NO: 54), goat (Capra hircus) β-casein protein (GenBank P33048; SEQ ID NO: 55), water buffalo (Bubalus bubalis) β-casein protein (GenBank Q9TSI0; SEQ ID NO: 56), and bovine (Bos taurus) β-casein protein (GenBank AGT56763.1; SEQ ID NO: 17), and. FIG. 23B shows results of the percent identity matrix based on the multiple sequence alignment analysis.

FIG. 24A shows results of the multiple sequence alignment analysis of human α-S1 casein protein (GenBank P47710; SEQ ID NO: 57), goat (Capra hircus) α-S1 casein protein (GenBank P18626; SEQ ID NO: 58), bovine (Bos taurus) α-S1 casein protein (GenBank P02662; SEQ ID NO: 59), and water buffalo (Bubalus bubalis) α-S1 casein protein (GenBank 062823; SEQ ID NO: 60). FIG. 24B shows results of the percent identity matrix based on the multiple sequence alignment analysis.

FIG. 25A shows results of the multiple sequence alignment analysis of goat (Capra hircus) α-S2 casein protein (GenBank P33049; SEQ ID NO: 61), bovine (Bos taurus) α-S2 casein protein (GenBank P02663; SEQ ID NO: 62), and water buffalo (Bubalus bubalis) α-S2 casein protein (GenBank CAR97769 and/or B6VPY3; SEQ ID NO: 63). FIG. 25B shows results of the percent identity matrix based on the multiple sequence alignment analysis.

### DETAILED DESCRIPTION

While the following terms are believed to be well understood by one of ordinary skill in the art, the following definitions are set forth to facilitate explanation of the presently disclosed subject matter. The following description includes information that may be useful in understanding the present disclosure. It is not an admission that any of the information provided herein is prior art or relevant to the presently claimed disclosures, or that any publication specifically or implicitly referenced is prior art.

### Definitions

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the element.

The term “bovine” means relating to or affecting an animal of the cattle group in the biological subfamily Bovinae, which includes a diverse group of 10 genera of cattle, bison, African buffalo, the water buffalo, the yak, and the four-horned and spiral-horned antelopes. See, e.g., Bovine Genomics, James E. Womack (editor), 2012, Wiley-Blackwell.

The term “bovine milk protein,” “or milk protein” or “proteins normally present in bovine milk” are synonymous as used herein and each refer to one or more proteins, or biologically active fragments thereof, found in normal bovine milk, including, without limitation, of α-S1 casein, α-S2 casein, β-casein, κ-casein, α-lactalbumin, β-lactoglobulin, serum albumin, lactoferrin, lysozyme, lactoperoxidase, immunoglobulin-A, lipase, and biologically active fragments thereof. β-casein includes A1, A2, A3, B, C, D, E, F, H1, H2, I, and G genetic variants of the beta-casein proteins.

The term “casein” refers to a protein that is derived from the milk of many species and the name for a family of related phosphoproteins (αS1, αS2, β, κ). For detailed coverage for the chemistry of milk proteins, lipids and lactose, see, e.g., Advanced Dairy Chemistry, Volume 1A: Proteins: Basic Aspects, Fourth Edition, Paul L. H. McSweeney and Patrick F. Fox (editors), 2013, Springer; and, Advanced Dairy Chemistry, Volume 1B: Proteins: Applied Aspects, Paul L. H. McSweeney and James A. O'Mahony (editors), 2015, Springer.

The term “chimeric gene” or “heterologous nucleic acid construct”, as defined herein refers to a construct which has been introduced into a host and may include parts of different genes of exogenous or autologous origin, including regulatory elements. A chimeric gene construct for plant/seed transformation is typically composed of a transcriptional regulatory region (promoter) operably linked to a heterologous protein coding sequence, or, in a selectable marker heterologous nucleic acid construct, to a selectable marker gene encoding a protein conferring antibiotic resistance to transformed plant cells. A typical chimeric gene of the present disclosure, includes a transcriptional regulatory region inducible during seed development, a protein coding sequence, and a terminator sequence. A chimeric gene construct may also include a second DNA sequence encoding a signal peptide if secretion of the target protein is desired.

The term “cotyledon” means a type of seed leaf. The cotyledon contains the food storage tissues of the seed.

The term “cultivar” means a group of similar plants that by structural features and performance (i.e., morphological and physiological characteristics) can be identified from other varieties within the same species. Furthermore, the term “cultivar” variously refers to a variety, strain or race of plant that has been produced by horticultural or agronomic techniques. The terms cultivar, variety, strain and race are often used interchangeably by plant breeders, agronomists and farmers.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

The term “dicotyledon (dicot)” refers to a flowering plant whose embryos have two seed leaves or cotyledons. Examples of dicots include, but are not limited to, Arabidopsis, tobacco, tomato, potato, sweet potato, cassava, legumes including alfalfa, lima beans, pea, chick pea, soybean, carrot, strawberry, lettuce, oak, maple, walnut, rose, mint, squash, daisy, and cactus.

The term “gene”. As used herein, “gene” refers to the coding region and does not include nucleotide sequences that are 5′- or 3′- to that region. A functional gene is the coding region operably linked to a promoter or terminator. A gene can be introduced into a genome of a species, whether from a different species or from the same species, using transformation or various breeding methods.

The term “gene converted (conversion)” plant refers to plants which are developed by a plant breeding technique called backcrossing wherein essentially all of the desired morphological and physiological characteristics of a variety are recovered in addition to the one or more genes transferred into the variety via the backcrossing technique, via genetic engineering or via mutation. One or more loci may also be transferred.

The term “genetic rearrangement” refers to the re-association of genetic elements that can occur spontaneously in vivo as well as in vitro which introduce a new organization of genetic material. For instance, the splicing together of polynucleotides at different chromosomal loci, can occur spontaneously in vivo during both plant development and sexual recombination. Accordingly, recombination of genetic elements by non-natural genetic modification techniques in vitro is akin to recombination events that also can occur through sexual recombination in vivo. The insertion of a DNA insert into a plant genome, for instance, is an example of a genetic or genomic rearrangement.

The term “heterologous DNA” or “foreign DNA” refers to DNA which has been introduced into plant cells from another source, or which is from a plant source, including the same plant source, but which is under the control of a promoter or terminator that does not normally regulate expression of the heterologous DNA.

The term “heterologous protein” is a protein, including a polypeptide, encoded by a heterologous DNA.

The term “homologous sequences” or “homologs” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Mich.), AlignX, and Vector NTI (Invitrogen, Carlsbad, Calif.).

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the terms “introgression”, “introgressed” and “introgressing” refer to the process whereby genes of one species, variety or cultivar are moved into the genome of another species, variety or cultivar, by crossing those species. The crossing may be natural or artificial. The process may optionally be completed by backcrossing to the recurrent parent, in which case introgression refers to infiltration of the genes of one species into the gene pool of another through repeated backcrossing of an interspecific hybrid with one of its parents. An introgression may also be described as a heterologous genetic material stably integrated in the genome of a recipient plant.

The term “in frame” means that nucleotide triplets (codons) are translated into a nascent amino acid sequence of the desired recombinant protein in a plant cell. Specifically, the present disclosure contemplates a first nucleic acid linked in reading frame to a second nucleic acid, wherein the first nucleotide sequence is a gene and the second nucleotide is a promoter or similar regulatory element.

The term “integrate” refers to the insertion of a nucleic acid sequence from a selected plant species, or from a plant that is from the same species as the selected plant, or from a plant that is sexually compatible with the selected plant species, into the genome of a cell of a selected plant species. “Integration” refers to the incorporation of only native genetic elements into a plant cell genome. In order to integrate a native genetic element, such as by homologous recombination, the present disclosure may “use” non-native DNA as a step in such a process. Thus, the present disclosure distinguishes between the “use of” a particular DNA molecule and the “integration” of a particular DNA molecule into a plant cell genome.

The term “introduction” or “introduced” refers to the insertion of a nucleic acid sequence into a cell, by methods including infection, transfection, transformation or transduction and includes the incorporation of a nucleic acid sequence into a eukaryotic or prokaryotic cell where the nucleic acid sequence may be incorporated into the genome of the cell, converted into an autonomous replicon, or transiently expressed.

The term “isolated” refers to any nucleic acid or compound that is physically separated from its normal, native environment. The isolated material may be maintained in a suitable solution containing, for instance, a solvent, a buffer, an ion, or other components, and may be in purified, or unpurified, form.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the average person skilled in molecular-biological techniques which techniques are for instance described in Lefebvre and Chevre, 1995; Lorez and Wenzel, 2007, Srivastava and Narula, 2004, Meksem and Kahl, 2005, Phillips and Vasil, 2001. General information concerning AFLP technology can be found in Vos et al. (1995, AFLP: a new technique for DNA fingerprinting, Nucleic Acids Res. 1995 November 11; 23(21): 4407-4414).

The term “monocotyledon (monocot) means a flowering plant whose embryos have one cotyledon or seed leaf. Examples of monocots include, but are not limited to turf grass, maize (corn), rice, oat, wheat, barley, sorghum, orchid, iris, lily, onion, palm, and duckweed.

The terms “native” and “wild-type” relative to a given plant trait or phenotype refers to the form in which that trait or phenotype is found in the same variety of plant in nature.

The “non-human mammals” of the disclosure comprise all non-human mammals capable of producing a “transgenic non-human mammal” having a “desirable phenotype”. Such mammals include non-human primates, murine species, bovine species, canine species, etc. Preferred non-human animals include bovine, porcine and ovine species, most preferably bovine species.

The term “nutritionally enhanced food” refers to a food, typically a processed food, to which a bovine milk protein has been added, in an amount effective to confer some health benefit, such as improved gut health, resistance to pathogenic bacteria, or iron transport, to a human consuming the food.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, “operably linked” elements, e.g., enhancers, do not have to be contiguous. Linking is accomplished by ligation at convenient restriction sites. If such sites do not exist, the synthetic oligonucleotide adaptors or linkers are used in accordance with conventional practice.

The term “plasmid” refers to a circular double-stranded (ds) DNA construct used as a cloning vector, and which forms an extrachromosomal self-replicating genetic element in many bacteria and some eukaryotes.

The term “plant” includes reference to whole plants, plant organs, plant tissues, and plant cells and progeny of same, but is not limited to angiosperms and gymnosperms such as Arabidopsis, potato, tomato, tobacco, alfalfa, lettuce, carrot, strawberry, sugarbeet, cassava, sweet potato, soybean, lima bean, pea, chick pea, maize (corn), turf grass, wheat, rice, barley, sorghum, oat, oak, eucalyptus, walnut, palm and duckweed as well as fern and moss. Thus, a plant may be a monocot, a dicot, a vascular plant reproduced from spores such as fern or a non-vascular plant such as moss, liverwort, hornwort and algae. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields. Expression of an introduced leader, trailer or gene sequences in plants may be transient or permanent. A “selected plant species” may be, but is not limited to, a species of any one of these “plants.”

The term “non-vascular plant” refers to a plant without a vascular system consisting of xylem and phloem, but many non-vascular plants has simpler tissues that are specialized for internal transport of water. Mosses and leafy liverworts have structures that look like leaves, but are not true leaves because they are single sheets of cells with no stomata, no internal air spaces and have no xylem or phloem. These organisms an elementary cuticle which was important in the evolution of land plants. All land plants have a life cycle with an alternation of generations between a diploid sporophyte and a haploid gametophyte, but in all non-vascular land plants the gametophyte generation is dominant. In these plants, the sporophytes grow from and are dependent on gametophytes for taking in water and mineral nutrients and for provision of photosynthate, the products of photosynthesis. Non-vascular plants include two distantly related groups: 1) Bryophytes, which is further categorized as three separate land plant Divisions, namely Bryophyta (mosses), Marchantiophyta (liverworts), and Anthocerotophyta (hornworts). In all bryophytes, the primary plants are the haploid gametophytes, with the only diploid portion being the attached sporophyte, consisting of a stalk and sporangium. Because these plants lack lignified water-conducting tissues, they can't become as tall as most vascular plants. 2) Algae, especially the green algae, which consists of several unrelated groups. Only those groups of algae included in the Viridiplantae are still considered relatives of land plants.

The term “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots”.

The term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

The term “plant species” refers to the group of plants belonging to various officially named plant species that display at least some sexual compatibility.

The term “plant transformation and cell culture” broadly refers to the process by which plant cells are genetically modified and transferred to an appropriate plant culture medium for maintenance, further growth, and/or further development.

The term “plant-derived food ingredients” refers to plant-derived food stuff, typically grain, but also including, separately, lectins, gums, sugars, plant-produced proteins and lipids, that may be blended or combined, alone or in combination with one or more plant-derived ingredients, to form an edible food.

The term “plasmid” refers to a circular double-stranded (ds) DNA construct used as a cloning vector, and which forms an extrachromosomal self-replicating genetic element in many bacteria and some eukaryotes

The term “polypeptide” refers to a biopolymer compound made up of a single chain of amino acid residues linked by peptide bonds. The term “protein” as used herein may be synonymous with the term “polypeptide” or may refer, in addition, to a complex of two or more polypeptides.

The term “promoter” or a “transcription regulatory region” or refers to nucleic acid sequences that influence and/or promote initiation of transcription. Promoters are typically considered to include regulatory regions, such as enhancer or inducer elements. The promoter will generally be appropriate to the host cell in which the target gene is being expressed. The promoter, together with other transcriptional and translational regulatory nucleic acid sequences (also termed “control sequences”), is necessary to express any given gene. In general, the transcriptional and translational regulatory sequences include, but are not limited to, promoter sequences, ribosomal binding sites, transcriptional start and stop sequences, translational start and stop sequences, and enhancer or activator sequences.

The term “proteolysis” or “proteolytic” or “proteolyze” means the breakdown of proteins into smaller polypeptides or amino acids. Uncatalyzed hydrolysis of peptide bonds is extremely slow. Proteolysis is typically catalyzed by cellular enzymes called proteases, but may also occur by intra-molecular digestion. Low pH or high temperatures can also cause proteolysis non-enzymatically. Limited proteolysis of a polypeptide during or after translation in protein synthesis often occurs for many proteins. This may involve removal of the N-terminal methionine, signal peptide, and/or the conversion of an inactive or non-functional protein to an active one.

The term “purifying” is used interchangeably with the term “isolating” and generally refers to the separation of a particular component from other components of the environment in which it was found or produced. For example, purifying a recombinant protein from plant cells in which it was produced typically means subjecting transgenic protein containing plant material to biochemical purification and/or column chromatography.

The term “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. As used herein, the term describes proteins that have been produced following the transfer of genes into the cells of plant host systems. “Recombinant” also broadly describes various technologies whereby genes can be cloned, DNA can be sequenced, and protein products can be produced.

The term “regeneration” refers to the development of a plant from tissue culture.

The term “regulatory sequences” refers to those sequences which are standard and known to those in the art, that may be included in the expression vectors to increase and/or maximize transcription of a gene of interest or translation of the resulting RNA in a plant system. These include, but are not limited to, promoters, peptide export signal sequences, introns, polyadenylation, and transcription termination sites. Methods of modifying nucleic acid constructs to increase expression levels in plants are also generally known in the art (see, e.g. Rogers et al., 260 J Biol. Chem. 3731-38, 1985; Cornejo et al., 23 Plant Mal. Biol. 567: 81, 1993). In engineering a plant system to affect the rate of transcription of a protein, various factors known in the art, including regulatory sequences such as positively or negatively acting sequences, enhancers and silencers, as well as chromatin structure may have an impact. The present disclosure provides that at least one of these factors may be utilized in engineering plants to express a protein of interest. The regulatory sequences of the present disclosure are native genetic elements, i.e., are isolated from the selected plant species to be modified.

The term “selectable marker” is typically a gene that codes for a protein that confers some kind of resistance to an antibiotic, herbicide or toxic compound, and is used to identify transformation events. Examples of selectable markers include the streptomycin phosphotransferase (spt) gene encoding streptomycin resistance, the phosphomannose isomerase (pmi) gene that converts mannose-6-phosphate into fructose-6 phosphate; the neomycin phosphotransferase (npt) gene encoding kanamycin and geneticin resistance, the hygromycin phosphotransferase (hpt or aphiv) gene encoding resistance to hygromycin, acetolactate synthase (als) genes encoding resistance to sulfonylurea-type herbicides, genes coding for resistance to herbicides which act to inhibit the action of glutamine synthase such as phosphinothricin or basta (e.g., the bar gene), or other similar genes known in the art. Alternatively, the term “selectable marker” refers to a selection system to generate antibiotic-free transgenic plants, which can be bio-safe markers. Examples of selectable antibiotic-free markers include, but not limited to, visible colors induced by anthocyanin accumulation for plant transformation, β-Glucuronidase (GUS) and green fluorescent protein (GFP) as visual markers, and the antisense gene for glutamate 1-semialdehyde aminotransferase (GSA-AT) that may interrupt chlorophyll synthesis by repressing partially or completely GSA-AT gene expression.

The term “sample” includes a sample from a plant, a plant part, a plant cell, or from a transmission vector, or a soil, water or air sample.

The term “seed” is meant to encompass all seed components, including, for example, the coleoptile and leaves, radicle and coleorhiza, scutellum, starchy endosperm, aleurone layer, pericarp and/or testa, either during seed maturation and seed germination.

The term “seed in a form for use as a food or food supplement” includes, but is not limited to, seed fractions such as de-hulled whole seed, flour (seed that has been de-hulled by milling and ground into a powder) a seed protein extract (where the protein fraction of the flour has been separated from the carbohydrate fraction) and/or a purified protein fraction derived from the transgenic grain.

The term “self-crossing”, “self-pollinated” or “self-pollination” means the pollen of one flower on one plant is applied (artificially or naturally) to the ovule (stigma) of the same or a different flower on the same plant.

The term “sequence identity” means nucleic acid or amino acid sequence identity in two or more aligned sequences, aligned using a sequence alignment program.

The term “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

The term “substantially unpurified form”, as applied to milk proteins in an extract of plants and/or a part thereof, means that the protein or proteins present in the extract are present in an amount less than 50% by weight.

The term “therapeutic agent” refers to one or more milk proteins or transgenic plants expressing one or more milk proteins administered in an amount effective to achieve a therapeutic effect. The milk protein or plants may be administered in a natural, unmodified form, or purified. The milk protein or plants may a source for the purified therapeutic agent. The therapeutic agent may include any necessary excipients or formulations for administration.

The term “transformation” refers to the transfer of nucleic acid (i.e., a nucleotide polymer) into a cell. As used herein, the term “genetic transformation” refers to the transfer and incorporation of DNA, especially recombinant DNA, into a cell.

The term “transformation of plant cells” refers to a process by which DNA is stably integrated into the genome of a plant cell. “Stably” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

The term “transgene” refers to a gene that will be inserted into a host genome, comprising a protein coding region. In the context of the instant disclosure, the elements comprising the transgene are isolated from the host genome.

The term “transgenic plant” means a genetically modified plant which contains at least one transgene. The term refers to a plant that has incorporated exogenous nucleic acid sequences, i.e., nucleic acid sequences which are not present in the native (“untransformed”) plant or plant cell. Thus a plant having within its cells a heterologous polynucleotide is referred to herein as a “transgenic plant”. The heterologous polynucleotide can be either stably integrated into the genome, or can be extra-chromosomal. Preferably, the polynucleotide of the present disclosure is stably integrated into the genome such that the polynucleotide is passed on to successive generations. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. “Transgenic” is used herein to include any cell, cell line, callus, tissue, plant part or plant, the genotype of which has been altered by the presence of heterologous nucleic acids including those transgenics initially so altered as well as those created by sexual crosses or asexual reproduction of the initial transgenics.

The term “transformant” refers to a cell, tissue or organism that has undergone transformation. The original transformant is designated as “T0” or “T₀.” Selfing the T0 produces a first transformed generation designated as “T1” or “T₁.”

The terms “transformed”, “stably transformed” or “transgenic” with reference to a cell, preferably, a plant cell means the (plant) cell has a non-native (heterologous) nucleic acid sequence integrated into its genome which is maintained through one or more generations.

The term “transient” refers to a period of time that is long enough to permit isolation of protein from a suitable plant tissue. Protein expression is at suitably high levels within at least about 1 hour, at least about 2 hours, at least about 3 hours, at least about 6 hours, at least about 12 hours, at least 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days after introduction of the expression construct into plant tissue. In one aspect, suitably high levels are obtained within 3-7 or 5-10 days and more preferably within 3-5 or 5-7 days, after introduction of an expression construct into the plant tissue.

The term “transient expression” or “transiently expressed” refers to expression in cells in which a virus, a transgene, a chimeric gene, or a recombinant/heterologous DNA sequence is introduced by viral infection or by such methods as Agrobacterium-mediated transformation, electroporation, or biolistic bombardment, but not selected for its stable maintenance.

The term “vacuum infiltration”, as used herein, relates to a method that allows the penetration of pathogenic bacteria, e.g. Agrobacterium, into the intercellular or interstitial spaces. Physically, the vacuum generates a negative atmospheric pressure that causes the air spaces between the cells in the plant tissue to decrease. The longer the duration and the lower the pressure, the less air space there is within the plant tissue. A subsequent increase in the pressure allows the bacterial suspension used in the infiltration to relocate into the plant tissue, and causes the Agrobacterium cells to contact the plant cells inside the plant tissue.

The term “variety” refers to a subdivision of a species, consisting of a group of individuals within the species that are distinct in form or function from other similar arrays of individuals. Also, the term “variety” has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

The term “vascular plant”, also known as tracheophytes and also higher plants, refers to a large group of plants (c. 308,312 accepted known species) that are defined as those land plants that have lignified tissues (the xylem) for conducting water and minerals throughout the plant and a specialized non-lignified tissue (the phloem) to conduct products of photosynthesis. Vascular plants include the clubmosses, horsetails, ferns, gymnosperms (including conifers) and angiosperms (flowering plants). Scientific names for the group include Tracheophyta and Tracheobionta. Vascular plants are distinguished by two primary characteristics: 1) Vascular plants have vascular tissues which distribute resources through the plant. This feature allows vascular plants to evolve to a larger size than non-vascular plants, which lack these specialized conducting tissues and are therefore restricted to relatively small sizes. 2) In vascular plants, the principal generation phase is the sporophyte, which is usually diploid with two sets of chromosomes per cell. Only the germ cells and gametophytes are haploid. By contrast, the principal generation phase in non-vascular plants is the gametophyte, which is haploid with one set of chromosomes per cell. In these plants, only the spore stalk and capsule are diploid.

The term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art. Accordingly, an “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

The term “whey protein” as used herein is the collection of globular proteins isolated from whey, which is the liquid remaining after milk has been curdled and strained. Generally, the protein fraction in whey constitutes approximately 10% of the total dry solids in whey. Whey protein is typically a mixture of β-lactoglobulin, α-lactalbumin, bovine serum albumin, and immunoglobulins. Whey protein in this disclosure can be referred to individual whey protein component (e.g. β-lactoglobulin, α-lactalbumin, bovine serum albumin, and immunoglobulins). See, e.g., McSweeney and Fox, 2013; and, McSweeney and O'Mahony, 2015, both supra.

The terms “wild type” or “WT” as used herein refer to the phenotype and/or genotype of the typical form of a species as it occurs in nature and/or commercially. In some instances, these terms refer to the non-transgenic form of a specific plant or variety of plants or species of plants. For example, a WT rice is a rice plant that does not comprise a DNA sequence coding for a bovine milk protein, while the corresponding non-WT rice plant is a transgenic rice plant which has incorporated into its DNA a sequence coding for a bovine milk protein.

The term “2A sequence”, “2A system”, or “2A expression system”, used herein, refers to nucleic acid sequence encoding 2A peptide itself or nucleic acid sequence encoding 2A peptide in one or more expression vectors/constructs. The average length of 2A peptides is 18-22 amino acids. The designation “2A” refers to a specific region of picornavirus polyproteins and arose from a systematic nomenclature adopted by researchers. In foot-and-mouth disease virus (FMDV), a member of Picornaviridae family, a 2A sequence appears to have the unique capability to mediate cleavage at its own C-terminus by an apparently enzyme-independent, novel type of reaction. This sequence can also mediate cleavage in a heterologous protein context in a range of eukaryotic expression systems. The 2A sequence is inserted between two genes of interest, maintaining a single open reading frame. Efficient cleavage of the polyprotein can lead to co-ordinate expression of active two proteins of interest. Self-processing polyproteins using the FMDV 2A sequence could therefore provide a system for ensuring coordinated, stable expression of multiple introduced proteins in cells including plant cells.

The term “IRES sequence”, used herein, refers to IRES sequences that are inserted into vectors/constructs to allow for expression of two genes from a single vector. An internal ribosome entry site, abbreviated IRES, is a RNA element that allows for translation initiation in an cap-independent manner, as part of the greater process of protein synthesis.

The term “% homology” is used interchangeably herein with the term “% identity” and refers to the level of nucleic acid or amino acid sequence identity between two or more aligned sequences, when aligned using a sequence alignment program. For example, 70% homology means the same thing as 70% sequence identity determined by a defined algorithm, and accordingly a homologue of a given sequence has greater than 80% sequence identity over a length of the given sequence. Exemplary levels of sequence identity include, but are not limited to, 80, 85, 90 or 95% or more sequence identity to a given sequence, e.g., the coding sequence for lactoferrin, as described herein.

### Bovine Milk Protein

Milk, mainly bovine milk, consumed in populations throughout the world, is a major source of protein in human diets. Bovine milk typically comprises around 30 grams per litre of protein. Approximately 4% of milk accounts for milk proteins, which consist of about 80% caseins and 20% whey proteins. While major components of whey proteins are α-lactalbumin (α-LA) and β-lactoglobulin (β-LG), casein proteins are classified into major subclasses α-casein (αS1- and αS2-), β-casein, and κ-casein, which are arranged in micelles (Swaisgood, 1982; Rodriquez et al., 1985). Furthermore, minor components such as bovine serum albumin, free amino acids, immunoglobulins, and proteolyzed fragments are present in the total protein concentration of milk (Maas et al., 1997; Elgar et al., 2000).

Among numerous specific proteins in the bovine milk, the primary group of milk proteins are 3 or 4 caseins, α-casein (αS1- and αS2-), β-casein, and κ-casein, which are a family of phosphoproteins. Each casein is a distinct molecule, but similar in its structure. Caseins form a multi-molecular, granular structure called a casein micelle in which some enzymes, water, and salts, such as calcium and phosphorous, are present. The micellar structure of casein in milk is significant in terms of a mode of digestion of milk in the stomach and intestine and a basis for separating some proteins and other components from cow milk. In practice, casein proteins in bovine milk can be separated from whey proteins by centrifugation or microfilteration to precipitate the casein proteins or by breaking the micellar structure by partial hydrolysis of the protein molecules with proteolytic enzymes.

Among caseins that make up the largest component (80%) of the bovine milk protein, β-caseins make up about 37% of the caseins. In the past two decades the body of evidence implicating casein proteins, especially beta-caseins, in a number of health disorders has been growing.

So far there are 12 identified genetic variants of β-casein reported: A1, A2, A3, B, C, D, E, F, G, H1, H2, and I (Kaminski S et al, 2007). Especially, the β-caseins can be categorized as beta-casein A1 and beta-casein A2. These two proteins are the predominant beta-caseins in milk consumed in most human populations. A1 and A2 beta-casein are genetic variants of the beta-casein milk protein that differ by one amino acid. A histidine amino acid is located at position 67 of the 209 amino acid sequence of beta-casein A1, whereas a proline is located at the same position of beta-casein A2. So the histidine amino acid in beta-casein variant A1 is substituted by proline in beta-casein variant A2 (Kaminski S et al., 2007). This single amino acid difference is, however, critically important to the enzymatic digestion of beta-caseins in the gut. The presence of histidine at position 67 allows a protein fragment comprising seven amino acids, known as beta-casomorphin-7 (BCM-7), to be produced on enzymatic digestion. Thus, BCM-7 is a digestion product of beta-casein A1. In the case of beta-casein A2, position 67 is occupied by a proline which hinders cleavage of the amino acid bond at that location. Thus, BCM-7 is not a digestion product of beta-casein A2.

Other beta-casein variants, such as beta-casein B and beta-casein C, also have histidine at position 67, and other variants, such as A3, D and E, have proline at position 67. But these variants are found only in very low levels, or not found at all, in milk from cows of European origin.

Thus, in the context of this disclosure, the term beta-casein A1 refers to any beta-casein having histidine at position 67, and the term beta-casein A2 refers to any beta-casein having proline at position 67.

BCM-7 is an opioid peptide and can potently activate opioid receptors throughout the body. BCM-7 has the ability to cross the gastrointestinal wall and enter circulation enabling it to influence systemic and cellular activities via opioid receptors. BCM-7 produced from beta-casein A1 interacts with the human digestive system, internal organs, and brainstem. While no direct causal relationships have been demonstrated between BCM-7 and these diseases due to a wide range of contributing factors for each illness, BCM-7 has been linked to type 1 diabetes, heart disease, autism, and other serious non-communicable diseases. A link between the consumption of beta-casein A1 in milk and milk products and the incidence of certain health conditions including type I diabetes (WO 1996/014577), coronary heart disease (WO 1996/036239) and neurological disorders (WO 2002/019832). Though A1 and A2 are the most common forms identified in dairy cattle breeds, the potential health benefits of A2 variant has been testified in several studies.

In some embodiments, the present disclosure teaches that beta-casein protein is selected from the group consisting of A1, A2, A3, B, C, D, E, F, H1, H2, I, and G genetic variant. In some embodiments, the beta-casein protein is A1, A2, or A3, D, or E variant of the beta-casein. In some embodiments, the beta-casein protein is A1 and/or A2 variant. In other embodiments, the beta-casein protein is A2 variant. In other embodiments, the present disclosure teaches the transgenic plants comprising a recombinant DNA construct encoding alpha-S1 casein protein. In other embodiments, the present disclosure teaches the transgenic plants comprising a recombinant DNA construct encoding alpha-S2 casein protein. In other embodiments, the present disclosure teaches the transgenic plants comprising a recombinant DNA construct encoding beta-casein protein including A1 variants and A2 variants. In other embodiments, the present disclosure teaches the transgenic plants comprising a recombinant DNA construct encoding beta-casein A1 protein. In other embodiments, the present disclosure teaches the transgenic plants comprising a recombinant DNA construct encoding beta-casein A2 protein. In other embodiments, the present disclosure teaches the transgenic plants comprising a recombinant DNA construct encoding kappa-S1 casein protein.

The major whey proteins in cow milk are α-lactalbumin (α-LA) and β-lactoglobulin (β-LG). Other whey proteins are serum albumin (a serum protein), immunoglobulins (antibodies), and various enzymes (such as lactoferrin, lysozyme, lactoperoxidase, lipase etc.), hormones (such as growth hormones), nutrient transporters, growth factors, disease resistance factors, and others. When whey proteins are not fully digested fully in digestive organs, some of the whey proteins may induce a localized or systemic immune response, known as milk protein allergy. β-lactoglobulin has been most often thought to be a cause of milk protein allergy.

Among various enzymes in milk proteins, lactoferrin and lysozyme play a critical role in defensive immune system. Lactoferrin is found at high concentrations within specific granules of polymorphonuclear leukocytes. Lysozyme is known as a major component of the secretory granules of neutrophils and macrophages and is released at the site of infection in the earliest stages of the immune response.

### Recombinant DNA Constructs for Transient Expression and Stable Transformation of Mammalian Milk Proteins

The present disclosure uses expression vectors that are recombinant DNA constructs (or heterologous DNA constructs or expression DNA cassettes) in which a chimeric gene is included with associated upstream and downstream sequences. Generally, expression vectors are designed for working in plants, and placing a chimeric gene that is operably linked to a 5′ upstream transcriptional regulatory region such as a promoter and a 3′ downstream transcriptional termination region such as a terminator. In the expression vectors, the 5′ upstream transcriptional regulatory region including a promoter is operably linked to the nucleic acid sequence encoding a milk protein found in mammalian milk. Also, the 3′ downstream transcriptional termination region is operably linked to the nucleic acid sequence encoding a milk protein found in mammalian milk.

As used herein, mammalian milk can refer to milk derived from bovine, human, goat, sheep, camel, buffalo, water buffalo, dromedary, llama and any combination thereof. In the present disclosure, mammalian milk protein can be produced in plants. In some embodiments, mammalian milk can be milk selected from bovine, human, goat, sheep, camel, buffalo, water buffalo, dromedary, llama and any combination thereof. In some embodiments, a mammalian milk is a bovine milk. For examples of human milk proteins, and their nucleic acid and amino acid sequences, that can be used in the compositions and methods of the present invention, see, e.g., U.S. Patent Application Publication No. 2003/0074700A1.

Importantly, a chimeric gene is inserted into a suitable plant-transformation vector having (i) companion sequences at the upstream and/or downstream of the chimeric gene, which are of plasmid or viral origin and provide necessary characteristics to the vector to permit the vector to move DNA from bacteria to the desired plant host; (ii) a selectable marker sequence; and (iii) a 3′ downstream transcriptional termination region generally at the opposite end of the vector from the transcription initiation regulatory region. One of the suitable plant-transformation vectors is a binary vector pCambia 1305.1. In general, the pCambia vector provides features such a high copy number in E. coli for high DNA yields, pVS1 replicon for high stability in Agrobacterium, restriction sites designed for modular plasmid modifications and adequate poly-linkers for introducing a chimeric gene, bacterial selection with chloramphenicol or kanamycin, plant selection with hygromycin B or kanamycin, and simple means to construct translational fusions to gusA reporter genes.

### Expression Vectors for Plant Transformation: Promoters

A chimeric gene included in expression vectors must be driven by nucleotide sequence comprising a transcriptional regulatory element, such as a promoter. Several types of promoters are now well known in the transformation arts, as are other regulatory elements that can be used alone or in combination with promoters.

A promoter can be a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain organs, such as leaves, roots, flowers, seeds and tissues such as fibers, xylem vessels, tracheids, or sclerenchyma. Such promoters are referred to as “tissue-preferred.” Promoters which initiate transcription only in certain tissue are referred to as “tissue-specific.” A “cell-type” specific promoter primarily drives expression in certain cell types in one or more organs, for example, vascular cells in leaves, roots, flowers, or seeds. An “inducible” promoter is a promoter which is under environmental control. Examples of environmental conditions that may affect transcription by inducible promoters include anaerobic conditions or the presence of light. Tissue-specific, tissue-preferred, cell-type specific, and inducible promoters constitute the class of “non-constitutive” promoters. A “constitutive” promoter is a promoter which is active under most environmental conditions.

Constitutive Promoters—

A constitutive promoter is operably linked to a gene for expression in plants or the constitutive promoter is operably linked to a nucleotide sequence encoding a signal sequence which is operably linked to a gene for expression in plants. Many different constitutive promoters can be utilized in the instant disclosure. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)). The ALS promoter, XbaI/NcoI fragment 5′ to the Brassica napus ALS3 structural gene (or a nucleotide sequence similarity to said XbaI/NcoI fragment), represents a particularly useful constitutive promoter. See PCT application WO96/30530.

In some embodiments, the constitutive promoter is a 35S promoter that is fused with a coding region of gene of interest. In some embodiments, the gene of interest comprises a nucleic acid sequence and/or a functional fragment thereof is a coding sequence for the bovine milk protein selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.

In some embodiments, the present disclosure provides constitutive promoters that are derived from dicot and/or monocot including, but not limited to, soybean, lima bean, Arabidopsis, tobacco and rice. In some embodiments, the presents disclosure provides promoters that are the most active in soybean.

New soybean (Glycine max (L.) Merr.) promoters, including but not limited to a soybean polyubiquitin (Gmubi) promoter, a soybean heat shock protein 90-like (GmHSP90L) promoter, a soybean Ethylene Response Factor (GmERF) promoter, have been well known to give strong constitutive expression, compared with the cauliflower mosaic virus 35S (CaMV35S) promoter, used as an expression standard. See, for examples, Chiera, J. M et al, (2007), Plant Cell Reports, 26(9), 1501-1509; Hernandez-Garcia et al, (2009), Plant cell reports, 28(5), 837-849; and Hernandez-Garcia et al, (2010), RUC plant biology, 10(1), 237, each of which is expressly incorporated herein by reference in their entirety.

In some embodiments, active constitutive soybean promoters are derived from GmScreamM1, GmScreamM4, GmScreamM8 genes (Zhang et al, Plant Science 241:189-198 (2015)) and GmubiXL genes (De La Torre and Finer, Plant Cell Reports 34:111-120 (2015)). In some embodiments, the present disclosure provides active constitutive soybean promoters comprise a GmScreamM1 promoter, a GmScreamM4 promoter, a GmScreamM8 promoter and a GmubiXL promoter. In some embodiments, the active constitutive soybean promoters disclosed above can be further modified with nucleotide substitution, addition and/or deletion for enhancing promoter activity. In other embodiments, the present disclosure provide a modified version of GmSM8, which is GmSM8-1 (SEQ ID NO:49).

In other embodiments, the most active constitutive soybean promoters disclosed herein show at least 1.1 folds, at least 1.2 folds, at least 1.3 folds, at least 1.4 folds, at least 1.5 folds, at least 1.6 folds, at least 1.7 folds, at least 1.8 folds, at least 1.9 folds, at least 2 folds, at least 3 folds, as least 4 folds, as least 5 folds, as least 6 folds, as least 7 folds, as least 8 folds, as least 9 folds, as least 10 folds, as least 11 folds, as least 12 folds, as least 13 folds, as least 14 folds, at least 15 folds or at least 20 folds higher expression than the 35S promoter in most of the tissues. In some embodiments, the tissues that have evaluated and/or tested for promoter activity are proliferative embryoenic tissues, procambium, vascular tissues, root tips, young embryo, mature embryo and the like. The promoters regulating highly expressing soybean genes are well known to those of ordinary skill in the art. See, for example, Zhang et al, Plant Science 241:189-198 (2015); and De La Torre and Finer, Plant Cell Reports 34:111-120 (2015); each of which is expressly incorporated herein by reference in their entirety.

In some embodiments, active constitutive soybean promoters including a GmScreamM1 (GmSM1) promoter (SEQ ID NO:46), a GmScreamM4 (GmSM4) promoter (SEQ ID NO:47), a GmScreamM8 (GmSm8) promoter (SEQ ID NO:48) and are identified, cloned and fused with a coding region of gene of interest. In some embodiments, active constitutive soybean promoters including a GmSM8-1 promoter (SEQ ID NO:49), in which nucleotide mismatches are introduced to a GmSM8 promoter (SEQ ID NO:48), is identified, cloned and fused with a coding region of gene of interest. In some embodiments, the gene of interest comprises a nucleic acid sequence and/or a functional fragment thereof is a coding sequence for the bovine milk protein selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase. In further embodiments, a nucleic acid sequence and/or a functional fragment thereof is a codon-optimized sequence selected from the group consisting of α-S1 casein, α-S2 casein, β-casein, κ-casein, α-lactalbumin, β-lactoglobulin, and lysozyme.

In some embodiments, the present disclosure provides nucleic acid sequences having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID No:46, SEQ ID No:47, SEQ ID No:48, and SEQ ID No:49.

In some embodiments, the constitutive soybean promoters disclosed above (e.g. GmSM1, GmSM4, GmSM8, GmSM8-1,) are adopted for driving full-length versions of transgenes and/or truncated versions of transgenes that do not containing signal peptide sequence. The transgenes under the control of the constitutive soybean promoters encode milk proteins provided in the present disclosure, and are fused with selectable markers such as GUS, GFP, and His-tag. In further embodiments, when the truncated versions of transgenes are driven by the constitutive soybean promoters, signal peptide-coding DNA sequences disclosed herein (including GmSM1, GmSM4, GmSM8, GmSM8-1, GmubiXL signal peptide) can be added to lead recombinant milk proteins to the purposed destination in which the proteins should be expressed.

Tissue-Specific or Tissue-Preferred Promoters—

A tissue-specific promoter is operably linked to a gene for expression in plants. Optionally, the tissue-specific promoter is operably linked to a nucleotide sequence encoding a signal sequence which is operably linked to a gene for expression in plants. Plants transformed with a gene of interest operably linked to a tissue-specific promoter produce the protein product of the transgene exclusively, or preferentially, in a specific tissue. Any tissue-specific or tissue-preferred promoter can be utilized in the instant disclosure. Exemplary tissue-specific or tissue-preferred promoters include, but are not limited to, a root-preferred promoter, such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from light-harvesting chlorophyll a/b binding protein (cab) or stromal RuVPC/Oase (rbc) (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from pollen-specific maize gene Zm13 (Hamilton et al., Plant. Mol. Biol. 18:211-218 (1992)).

In some embodiments, the present disclosure provides tissue-specific or tissue-preferred promoters present in dicot and/or monocot. In some embodiments, tissue-specific or tissue-preferred promoters are derived from dicot and/or monocot including, but not limited to, soybean, lima bean, Arabidopsis, tobacco and rice.

In some embodiments, the present disclosure provides promoters that are highly active in soybean developing seeds. In some embodiments, the active soybean tissue-specific promoters are derived from seed-preferentially expressed genes as described in Table 1. In some embodiments, the soybean tissue-specific promoters can be the soybean seed-specific promoters. The seed-specific promoters include, but being not limited to, AR-Pro1 promoter, AR-Pro2 promoter, AR-Pro3 promoter, AR-Pro4 promoter, AR-Pro5 promoter, AR-Pro6 promoter, AR-Pro7 promoter, AR-Pro8 promoter, and AR-Pro9 promoter. In other embodiments, the seed-specific promoters disclosed herein can be in dicot and monocot plants disclosed in this disclosure.

In the plant seeds, the seed-specific promoters provided in this disclosure may be equal and/or comparable in strength to the constitutive GmScreamM8 and GmubiXL promoters disclosed above, based on GFP expression, which reflects protein deposition. In other embodiments, the seed-specific promoters disclosed herein show at least 1.1 folds, at least 1.2 folds, at least 1.3 folds, at least 1.4 folds, at least 1.5 folds, at least 1.6 folds, at least 1.7 folds, at least 1.8 folds, at least 1.9 folds, at least 2 folds, at least 3 folds, as least 4 folds, as least 5 folds, as least 6 folds, as least 7 folds, as least 8 folds, as least 9 folds, as least 10 folds, as least 11 folds, as least 12 folds, as least 13 folds, as least 14 folds, at least 15 folds or at least 20 folds higher expression than the 35S promoter in developing and/or mature seeds of plants including dicot and monocot. The promoters regulating highly expressing soybean genes in a tissue-specific manner are well known to those of ordinary skill in the art. See, for example, Gunadi et al, Plant Cell, Tissue and Organ Culture 127:145-160, (2016); which is incorporated herein by reference in their entirety.

TABLE 1
Seed-specific expression cassettes

| | Length of the | Gene ID | |
|---|---|---|---|
| | Cloned | (Williams | |
| Expression | Promoter | 82. a2. | |
| Cassette | Region (bp) | v1) | Gene Annotation |
| AR-Pro1 | 1384 | Glyma.03G163500 | 12S Seed storage protein |
| | | | CRA1-related |
| AR-Pro2 | 1387 | Glyma.08G116300 | Cysteine protease |
| | | | family C1-related |
| AR-Pro3 | 1490 | Glyma.13G123500 | 12S Seed storage protein |
| | | | CRA1-related |
| AR-Pro4 | 1482 | Glyma.10G037100 | 12S Seed storage protein |
| | | | CRA1-related |
| AR-Pro5 | 1594 | Glyma.01G095000 | Kunitz family trypsin |
| | | | and protease |
| | | | inhibitor protein-related |
| AR-Pro6 | 1500 | Glyma.08G341500 | Kunitz family trypsin |
| | | | and protease |
| | | | inhibitor protein-related |
| AR-Pro7 | 1535 | Glyma.02G012600 | Legume lectin domain |
| AR-Pro8 | 1510 | Glyma.20G148400 | Cupin, functional |
| | | | in storage of |
| | | | nutritious substrates |
| AR-Pro9 | 1454 | Glyma.10G246300 | Cupin (Cupin_1) |

In some embodiments, seed-specific soybean promoters including a AR-Pro1 promoter (SEQ ID NO:28), a AR-Pro2 promoter (SEQ ID NO:30), a AR-Pro3 promoter (SEQ ID NO:32), a AR-Pro4 promoter (SEQ ID NO:34), a AR-Pro5 promoter (SEQ ID NO:36), a AR-Pro6 promoter (SEQ ID NO:38), a AR-Pro7 promoter (SEQ ID NO:40), a AR-Pro8 promoter (SEQ ID NO:42), and a AR-Pro9 promoter (SEQ ID NO:44), are identified, cloned and fused with a coding region of gene of interest. In some embodiments, the gene of interest comprises a nucleic acid sequence and/or a functional fragment thereof is a coding sequence for the bovine milk protein selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase. In further embodiments, a nucleic acid sequence and/or a functional fragment thereof is a codon-optimized sequence selected from the group consisting of α-S1 casein, α-S2 casein, β-casein, κ-casein, α-lactalbumin, β-lactoglobulin, and lysozyme.

In some embodiments, the present disclosure provides nucleic acid sequences having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID No:28, SEQ ID No:30, SEQ ID No:32, SEQ ID No:34, SEQ ID No:36, SEQ ID No:38, SEQ ID No:40, SEQ ID No:42, SEQ ID No:44.

In some embodiments, the present disclosure teaches sequence information of a AR-Pro1 signal peptide-coding DNA sequence (SEQ ID NO:29), a AR-Pro2 signal peptide-coding DNA sequence (SEQ ID NO:31), a AR-Pro3 signal peptide-coding DNA sequence (SEQ ID NO:33), a AR-Pro4 signal peptide-coding DNA sequence (SEQ ID NO:35), a AR-Pro5 signal peptide-coding DNA sequence (SEQ ID NO:37), a AR-Pro6 signal peptide-coding DNA sequence (SEQ ID NO:39), a AR-Pro7 signal peptide-coding DNA sequence (SEQ ID NO:41), a AR-Pro8 signal peptide-coding DNA sequence (SEQ ID NO:43), and a AR-Pro9 signal peptide-coding DNA sequence (SEQ ID NO:45).

In some embodiments, the present disclosure provides nucleic acid sequences having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID No:29, SEQ ID No:31, SEQ ID No:33, SEQ ID No:35, SEQ ID No:37, SEQ ID No:39, SEQ ID No:41, SEQ ID No:43, SEQ ID No:45.

In some embodiments, seed-specific and/or tissue-specific promoters disclosed above (e.g. AR-Pro1, AR-Pro2, AR-Pro3, AR-Pro4, AR-Pro5, AR-Pro6, AR-Pro7, AR-Pro8, and AR-Pro9 promoters) are used for driving full-length versions of transgenes and/or truncated versions of transgenes that do not containing signal peptide sequence. The transgenes driven by the seed-specific and/or tissue-specific promoters encode milk proteins provided in the present disclosure, and are fused with selectable markers such as GUS, GFP, and His-tag. In further embodiments, when the truncated versions of transgenes are controlled by seed-specific and/or tissue-specific promoters, signal peptide-coding DNA sequences disclosed herein (including AR-Pro1, AR-Pro2, AR-Pro3, AR-Pro4, AR-Pro5, AR-Pro6, AR-Pro7, AR-Pro8, and AR-Pro9 signal peptide) can be inserted before the truncated version of transgenes in the recombinant milk protein constructs.

In some embodiments, tissue-specific or tissue-preferred promoters includes seed specific promoters, which drive the nucleic acid sequence encoding signal peptide and chimeric fusion protein for tissue-specific expression.

Inducible Promoters—

An inducible promoter is operably linked to a gene for expression in plants. Optionally, the inducible promoter is operably linked to a nucleotide sequence encoding a signal sequence which is operably linked to a gene for expression in plants. With an inducible promoter the rate of transcription increases in response to an inducing agent. Any inducible promoter can be used in the instant disclosure. See Ward et al., Plant Mol. Biol. 22:361-366 (1993). Exemplary inducible promoters include, but are not limited to, that from the ACEI system which responds to copper (Mett et al., Proc. Natl. Acad. Sci. U.S.A. 90:4567-4571 (1993)); In2 gene from maize which responds to benzenesulfonamide herbicide safeners (Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. U.S.A. 88:0421 (1991)).

### Expression Vectors for Plant Transformation: Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized. The presence of a signal sequence directs a polypeptide to either an intracellular organelle or subcellular compartment or for secretion to the apoplast. Many signal sequences are known in the art. See, for example, Becker et al., Plant Mol. Bioi. 20:49 (1992); Close, P. S., Master's Thesis, Iowa State University (1993); Knox, C., et al., Plant Mol. Bioi. 9:3-17 (1987); Lerneret et al., Plant Physiol. 91:124-129 (1989); Frontes et al., Plant Cell. 3:483-496 (1991); Matsuoka et al., Proc. Natl. Acad. Sci. 88:834 (1991); Gould et al., J. Cell. Bioi. 108:1657 (1989); Creissen et al., Plant J. 2:129 (1991); Kalderon, et al., Cell. 39:499-509 (1984); Steifel, et al., Plant Cell 2:785-793 (1990).

Plant expression vectors, particularly binary vectors, and especially the minimally sized binary vectors according to any one of the preceding embodiments as described herein, which are functional in a plant cell and may be used within the method of the present disclosure, may further comprise a nucleotide sequence encoding a signal peptide that targets the newly expressed protein to a subcellular location. Signal peptides that may be used within such vector molecules comprise a vacuolar targeting sequence, a chloroplast targeting sequence, a mitochondrial targeting sequence, a sequence that induces the formation of protein bodies in a plant cell or a sequence that induces the formation of oil bodies in a plant cell.

In some embodiments, the targeting sequence is a signal peptide for import of a protein into the endoplasmic reticulum. Signal peptides are transit peptides that are located at the extreme N-terminus of a protein and cleaved co-translationally during translocation across the endoplasmatic reticulum membrane.

In other embodiments, the targeting sequence may be an endoplasmatic reticulum retention peptide. Endoplasmatic reticulum retention targeting sequences occur at the extreme C-terminus of a protein and can be a four amino acid sequence such as KDEL, HDEL or DDEL, wherein K is lysine, D is aspartic acid, E is glutamic acid, L is leucine and H is histidine.

In further embodiments, the targeting sequence may be a sequence that when fused to a protein results in the formation of non-secretory storage organelles in the endoplasmatic reticulum such as but not limited to those described in WO07/096,192, WO06/056483 and WO06/056484, which are incorporated herein by reference in their entirety. In certain embodiments of the disclosure, the targeting sequence can be a vacuolar targeting sequence, a chloroplast targeting sequence, a mitochondrial targeting sequence or any other sequence the addition of which results in a specific targeting of the protein fused there onto to a specific organelle within the plant or plant cell.

Further signal peptides can, for example, be predicted by the SignalP prediction tool (Emanuelsson et al., 2007, Nature Protocols 2: 953-971) and be used in this disclosure.

In some embodiments, the vectors provided in the disclosure and as defined in any one of the embodiments comprises in the T-DNA region a site-specific recombination site for site-specific recombination. In one embodiment, the site-specific recombination site is located downstream of the plant regulatory element. In another embodiment, the site-specific recombination site is located upstream of the plant regulatory element. In further embodiment, the recombination site is a LoxP site and part of a Cre-Lox site-specific recombination system. The Cre-Lox site-specific recombination system uses a cyclic recombinase (Cre) which catalyzes the recombination between specific sites (LoxP) that contain specific binding sites for Cre.

### Expression Vectors for Plant Transformation: Foreign Protein-Coding Genes

With transgenic plants developed according to the present disclosure, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981). In some embodiments, a transgenic plant provided for commercial production of foreign protein is soybean, tobacco, Arabidopsis, lima beans, rice, and duckweed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology CRC Press, Boca Raton 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

In one embodiment, the expression construct includes a transcription regulatory element, a promoter, which constitutively exhibits specifically upregulated activity of a chimeric gene. One example of such promoters is the 35S promoter from CaMV in the present disclosure.

The expression of the nucleic acid sequence encoding a bovine milk protein is of particular interest by a transcription initiation from region that is preferentially expressed in plant organs and/or tissues as well as constitutively expressed in whole plants or a part thereof. Examples of such preferential transcription initiation sequences include those sequences derived from sequences encoding plant genes expressed in organs and/or tissues including, but not limited to, seeds, leaves, stem, roots, inflorescences, and fruits.

In some cases, the promoter is derived from the same plant species as the plant cells into which the chimeric nucleic acid construct is to be introduced. Promoters for use in the instant disclosure will be typically derived from dicot plants such as soybean, lima beans, pea, chick pea, Arabidopsis, or tobacco as well as monocot plants such as duckweed, maize (corn), rice, barley, wheat, oat, rye, corm, millet, triticale or sorghum.

In some embodiments, the recombinant DNA construct contains the nucleic acid sequence coding for a heterologous protein, under the control of a promoter, which is such as a CaMV 35S promoter. The present disclosure provides polynucleotide sequences that code for bovine milk proteins including fragments of such bovine milk proteins, splicing variants, modified forms or functional equivalents thereof. Such nucleic acid sequences may be used in recombinant DNA constructs (also termed heterologous expression vectors), making bovine milk proteins expressed constitutively or tissue-preferentially or tissue-specifically or inducibly in appropriate host cells, plant organs/tissues, or whole plants.

A nucleic acid sequence encoding a functional fragment of a bovine milk protein may encode fragments or variations of bovine milk protein amino acid sequence that is modified by one or more amino acids from the native milk protein sequence, which are enclosed within the scope of the present disclosure. A “functional fragment of” bovine milk protein-encoding nucleic acid sequence means a “variant” bovine milk protein sequence which contains amino acid insertions or deletions, or both. A “variant” bovine milk protein-encoding nucleic acid sequence may encode a “variant” bovine milk protein sequence which contains a combination of any two or three of amino acid insertions, deletions, or substitution. The term “modified form” of a bovine milk protein similarly means a variant or derivative form of the native bovine milk protein or the nucleic acid sequence encoding such variants and/or modified form of the bovine milk.

In some embodiments, a functional fragment of a bovine milk contains at least one amino acid substitution, insertion, or deletion, which may take place at any residue within the sequence. However, such substitution, insertion, or deletion does not affect the biological and/or functional activity of the native bovine milk protein.

Furthermore, a nucleic acid sequence encoding a bovine milk protein may encode the same polypeptide as the reference polynucleotide or native sequence. However, the degeneracy of the genetic code causes the nucleic acid or polynucleotide sequences coding for the bovine milk protein to be altered by one or more bases from the reference or native nucleotide sequence.

Also, a nucleotide acid sequence encoding bovine milk proteins include “allelic variants” defined as an alternate form of a polynucleotide sequence which may have a substitution, deletion or addition of one or more nucleotides, which does not substantially change the biological function of the encoded polypeptide.

In some embodiments, the present disclosure teaches that a recombinant DNA construct comprises a nucleic acid encoding a bovine milk protein and/or a functional fragment thereof. In other embodiments, the present disclosure teaches that a recombinant DNA construct comprises a nucleic acid encoding a bovine milk protein and/or a functional fragment and/or a modified form thereof. In yet other embodiments, the present disclosure teaches that a recombinant DNA construct comprises a nucleic acid or polynucleotide sequence encoding a bovine milk protein, a variant, and/or allelic variants thereof.

In some embodiments, a recombinant DNA construct may contain a polynucleotide sequence coding for a given bovine milk protein, a variant or splice variant, a modified form, or a functional fragment thereof: (i) in combination with additional coding sequences; such as signal peptide; (ii) in combination with non-coding sequences, such as regulatory elements, promoter and terminator elements or 5′ and/or 3′ untranslated regions, for effective expression of the polynucleotide sequence in a host plant; (iii) in a vector or host environment in which the bovine milk protein coding sequence is a heterologous gene in isolation; and/or (iv) in isolation or (v) in synthesis.

In some embodiments, a recombinant DNA construct may contain the nucleic acid sequence which encodes the entire bovine milk protein, or a portion thereof. For example, a highly conserved portion of bovine milk protein sequences can be used for construction of heterologous expression cassettes and/or vectors.

In some embodiments, the present disclosure provides nucleic acid sequences encoding κ-casein, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:1. In some embodiments, a codon-optimized nucleic acid sequence encoding κ-casein has the nucleic acid sequence of SEQ ID NO:1.

In some embodiments, the present disclosure provides nucleic acid sequences encoding κ-casein without signal peptide, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:2. In some embodiments, a codon-optimized nucleic acid sequence encoding κ-casein without signal peptide has the nucleic acid sequence of SEQ ID NO:2.

In some embodiments, the present disclosure provides nucleic acid sequences encoding β-casein, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:3. In some embodiments, a codon-optimized nucleic acid sequence encoding β-casein has the nucleic acid sequence of SEQ ID NO:3.

In some embodiments, the present disclosure provides nucleic acid sequences encoding β-casein without signal peptide, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:4. In some embodiments, a codon-optimized nucleic acid sequence encoding β-casein without signal peptide has the nucleic acid sequence of SEQ ID NO:4.

In some embodiments, the present disclosure provides nucleic acid sequences encoding α-S1 casein, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:9. In some embodiments, a codon-optimized nucleic acid sequence encoding α-S1 casein has the nucleic acid sequence of SEQ ID NO:9.

In some embodiments, the present disclosure provides nucleic acid sequences encoding α-S2 casein, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:10. In some embodiments, a codon-optimized nucleic acid sequence encoding α-S2 casein has the nucleic acid sequence of SEQ ID NO:10.

In some embodiments, the present disclosure provides nucleic acid sequences encoding α-lactalbumin, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:19. In some embodiments, a codon-optimized nucleic acid sequence encoding α-lactalbumin has the nucleic acid sequence of SEQ ID NO:19.

In some embodiments, the present disclosure provides nucleic acid sequences encoding β-lactoglobulin, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:20. In some embodiments, a codon-optimized nucleic acid sequence encoding β-lactoglobulin has the nucleic acid sequence of SEQ ID NO:20.

In some embodiments, the present disclosure provides nucleic acid sequences encoding lysozyme, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:21. In some embodiments, a codon-optimized nucleic acid sequence encoding lysozyme has the nucleic acid sequence of SEQ ID NO:21.

In some embodiments, the present disclosure provides 2A and/or IRES sequences to simultaneously express at least two nucleic acid sequences encoding bovine milk proteins including α-S1 casein, α-S2 casein, β-casein, κ-casein, α-lactalbumin, β-lactoglobulin, serum albumin, lactoferrin, lysozyme, lactoperoxidase, immunoglobulin-A, and lipase. In some embodiments, the bovine milk proteins are α-S1 casein, α-S2 casein, β-casein, κ-casein, α-lactalbumin, β-lactoglobulin, and lysozyme. In some embodiments, the present disclosure provides constructs and/or vectors expressing at least two casein genes using 2A system. In some embodiments, the at least two casein genes that are engineered by 2A system are selected from codon-optimized nucleic acid sequences that encode α-S1 casein, α-S2 casein, β-casein, κ-casein, α-lactalbumin, β-lactoglobulin, and lysozyme. In other embodiments, the present disclosure provides a process of generating constructs and/or vectors expressing at least two casein gene using 2A system. The application of two bicistronic systems involving 2A and/or IRES sequences for genetic engineering is well known to those of ordinary skill in the art. See, for example, Kim et al, PLos One, 7(10): e48287, (2012); Ha et al. Plant Biotechnology Journal 8:928-938, (2010); and Halpin et al., Plant Journal, 17(4):453-459, (1999); each of which is incorporated herein by reference in their entirety.

In some embodiments, the present disclosure provides nucleic acid sequences encoding 2A peptide, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:13. In some embodiments, a codon-optimized nucleic acid sequence encoding 2A peptide has the nucleic acid sequence of SEQ ID NO:13.

### Expression Vectors for Plant Transformation: Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or a herbicide, or genes that encode an altered target which is insensitive to the inhibitor. A few positive selection methods are also known in the art.

One commonly used selectable marker gene for plant transformation is the neomycin phosphotransferase II (nptII) gene, isolated from transposon Tn5, which, when placed under the control of plant regulatory signals, confers resistance to kanamycin (Fraley et al., Proc. Natl. Acad. Sci. U.S.A., 80:4803 (1983)). Another commonly used selectable marker gene is the hygromycin phosphotransferase gene which confers resistance to the antibiotic hygromycin (Vanden Elzen et al., Plant Mol. Biol., 5:299 (1985)).

Additional selectable marker genes of bacterial origin that confer resistance to antibiotics include gentamycin acetyl transferase, streptomycin phosphotransferase, and aminoglycoside-3′-adenyl transferase, the bleomycin resistance determinant (Hayford et al., Plant Physiol. 86:1216 (1988), Jones et al., Mol. Gen. Genet., 210:86 (1987), Svab et al., Plant Mol. Biol. 14:197 (1990), and Hille et al., Plant Mol. Biol. 7:171 (1986)). Other selectable marker genes confer resistance to herbicides such as glyphosate, glufosinate or bromoxynil (Comai et al., Nature 317:741-744 (1985), Gordon-Kamm et al., Plant Cell 2:603-618 (1990) and Stalker et al., Science 242:419-423 (1988)).

Selectable marker genes for plant transformation that are not of bacterial origin include, for example, mouse dihydrofolate reductase, plant 5-enolpyruvylshikimate-3-phosphate synthase and plant acetolactate synthase (Eichholtz et al., Somatic Cell Mol. Genet. 13:67 (1987), Shah et al., Science 233:478 (1986), and Charest et al., Plant Cell Rep. 8:643 (1990)).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include beta-glucuronidase (GUS), beta-galactosidase, luciferase, and chloramphenicol acetyltransferase (Jefferson, R. A., Plant Mol. Biol. Rep. 5:387 (1987), Teeri et al., EMBO J. 8:343 (1989), Koncz et al., Proc. Natl. Acad. Sci U.S.A. 84:131 (1987), and DeBlock et al., EMBO J. 3:1681 (1984). Another approach to the identification of relatively rare transformation events has been use of a gene that encodes a dominant constitutive regulator of the Zea mays anthocyanin pigmentation pathway (Ludwig et al., Science 247:449 (1990)).

In vivo methods for visualizing GUS activity that do not require destruction of plant tissue are also available. However, these in vivo methods for visualizing GUS activity have not proven useful for recovery of transformed cells because of low sensitivity, high fluorescent backgrounds and limitations associated with the use of luciferase genes as selectable markers.

A gene encoding Green Fluorescent Protein (GFP) has been utilized as a marker for gene expression in prokaryotic and eukaryotic cells (Chalfie et al., Science 263:802 (1994)). GFP and mutants of GFP may be used as screenable markers.

In some embodiments, the vector contains a selectable, screenable, or scoreable marker gene. These genetic components are also referred to herein as functional genetic components, as they produce a product that serves a function in the identification of a transformed plant, or a product of agronomic utility. The DNA that serves as a selection or screening device may function in a regenerable plant tissue to produce a compound that would confer upon the plant tissue resistance to an otherwise toxic compound. A number of screenable or selectable marker genes are known in the art and can be used in the present disclosure. Genes of interest for use as a marker would include but are not limited to GUS, green fluorescent protein (GFP), luciferase (LUX), among others. In certain embodiments, the vector comprises an aadA gene with associated regulatory elements encoding resistance to spectinomycin in plant cells. In some embodiments, the aadA gene comprises a chloroplast transit peptide (CTP) sequence that directs the transport of the AadA gene product to the chloroplast of a transformed plant cell. In other embodiments, the vector comprises a spectinomycin resistance gene with appropriate regulatory elements designed for expression in a bacterial cell, such as an Agrobacterium cell, so that the selection reagent may be added to a co-cultivation medium, and allowing obtention of transgenic plants for instance without further use of the selective agent after the co-culture period. In other embodiments, the Bar gene has been widely used as a selectable marker for plant transformation. Glufosinate (also known as phosphinothricin and often sold as an ammonium salt) is a naturally occurring broad-spectrum systemic herbicide produced by several species of Streptomyces soil bacteria. Plants also metabolize bialaphos, another naturally occurring herbicide, directly into glufosinate. The compound irreversibly inhibits glutamine synthetase, an enzyme necessary for the production of glutamine and for ammonia detoxification, giving it antibacterial, antifungal and herbicidal properties. Application of glufosinate to plants leads to reduced glutamine and elevated ammonia levels in tissues, halting photosynthesis, resulting in plant death. Transgenic cells and plants expressing this gene are resistant to the herbicides Basta (registered in Europe), Bialaphos (registered in Japan) and Ignite (registered in the USA). In other embodiments, the present disclosure also teaches the use of selectable markers including bar gene conferring resistance to glufosinate (also known as phosphinothricin) or bialaphos, and the aadA gene conferring resistance to spectinomycin and streptomycin.

A new visual selectable marker gene that confers tolerance to multiple abiotic stresses in transgenic tomato is known such as Jin F. et al, (2012) Transgenic Res 21:1057-1070, which is expressly incorporated herein by reference in its entirety.

In some embodiments, the present disclosure teaches the use of a selectable marker, including GUSplus™ and GFP genes.

In some embodiments, the present disclosure provides nucleotide sequence information of GUS gene as a selection marker, which is fused with 6×His-tag.

In some embodiments, the present disclosure provides nucleotide sequence information of GFP gene as a selection marker, which is fused with 6×His-tag. In some embodiments, the disclosure teaches nucleic acid sequences encoding GFP and 6×His-tag, and/or functional fragments and variations thereof comprising a nucleic acid sequence that shares at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID No:14. In some embodiments, the nucleic acid sequence encoding GFP and 6×His-tag has the nucleic acid sequence of SEQ ID NO:14.

### Codon Optimization

Nucleic acid sequences which encode substantially the same or a functionally equivalent amino acid sequence may be used for cloning and expressing a given bovine milk protein, as described herein by the codon-optimized coding sequences. The degeneracy of codons is the redundancy of the genetic code, which is explained as the multiplicity of three-base pair codon combinations that direct specific amino acid. The degeneracy of the genetic codon offers a feature of fault-tolerance for mutations in sequence. For the practice of the present disclosure, the degeneracy of the genetic code allows for multiple nucleic acid sequences encoding a given bovine milk protein to be generated. For example, the triplet GAA and GAG specifies the amino acid glutamic acid, which is clearly different in the third position. The amino acid serine is specified by TCA, TCG, TCC, TCU, AGT, AGC, which are substantially different in the first, second, and third position. Such degeneracies in the nucleotide sequence variants, but coding for the same codon can be applied in the same way as described herein for a given bovine milk protein-encoding nucleic acid sequence.

In some embodiments, it may be advantageous for a person in ordinary skill in the art to use a bovine milk protein-encoding polynucleotide sequences possessing non-naturally occurring codons. The patterns of codon usage differ in the case of eukaryotic hosts (Murray et al., 1989; Campbell et al. 1990). It has been shown that production of recombinant protein in transgenic barley grain was enhanced by codon optimization of the gene (Horvath et al., 2000; Jensen et al., 1996). Codon can be preferred to generate high level of recombinant RNA transcripts that may be more stable for a longer half-life, than naturally-occurring transcripts and/or to consequently increase the bovine milk protein expression level. Codon-optimized sequences are utilized to practice the present disclosure.

In some embodiments, the present disclosure provides sequence information of four types of casein protein (α-S1, α-S2, β-, κ-). α-S1-casein protein sequence is deposited as GenBank accession No. ACG63494.1 and α-S2-casein protein sequence is deposited as GenBank accession No. NP 776953.1. In other embodiments, β-casein protein sequence is deposited as GenBank accession No. AGT56763.1. In further embodiments, κ-casein protein sequence is deposited as GenBank accession No. AAQ87923.1.

In some embodiments, the present disclosure provides sequence information of whey protein including α-lactalbumin, β-lactoglobulin, and lysozyme. α-lactalbumin protein sequence is deposited as GenBank accession No. NP_776803.1, β-lactoglobulin protein sequence deposited as GenBank accession No. NP_776354.2 and lysozyme protein sequence deposited as GenBank accession No. NP_001071297.1.

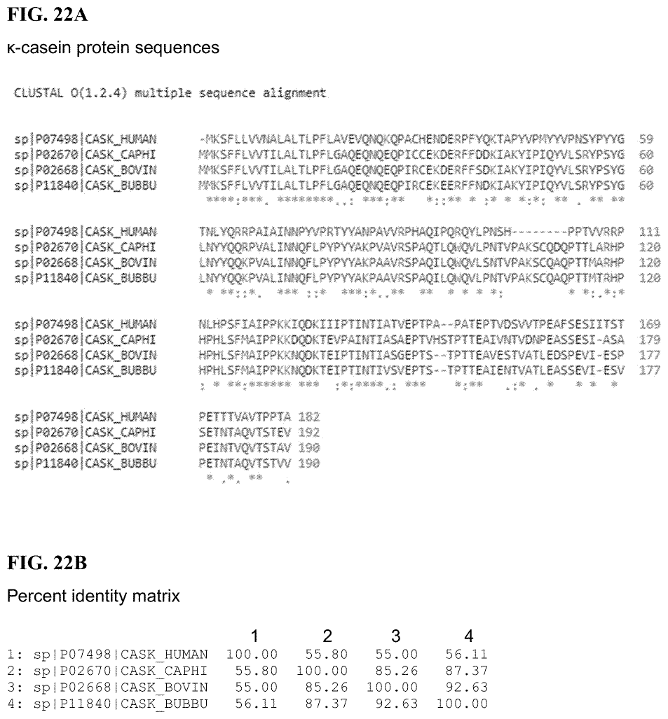

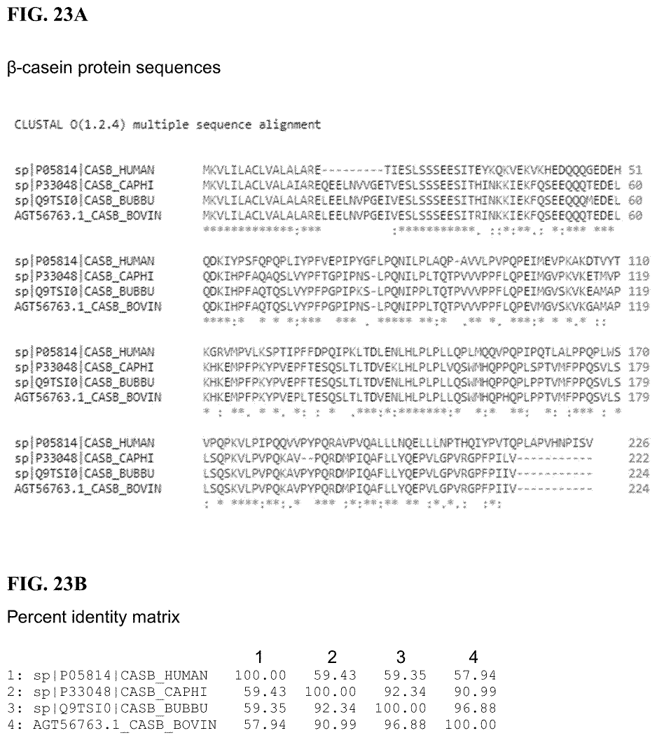

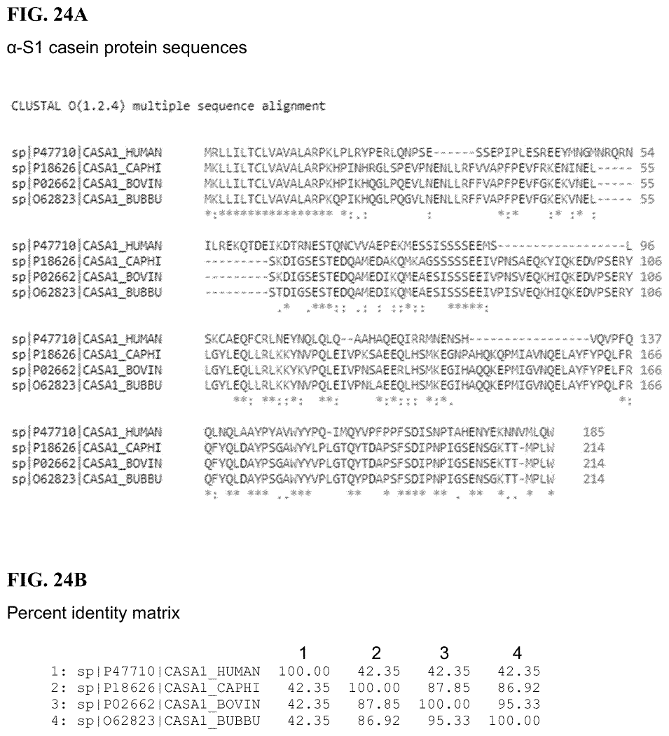

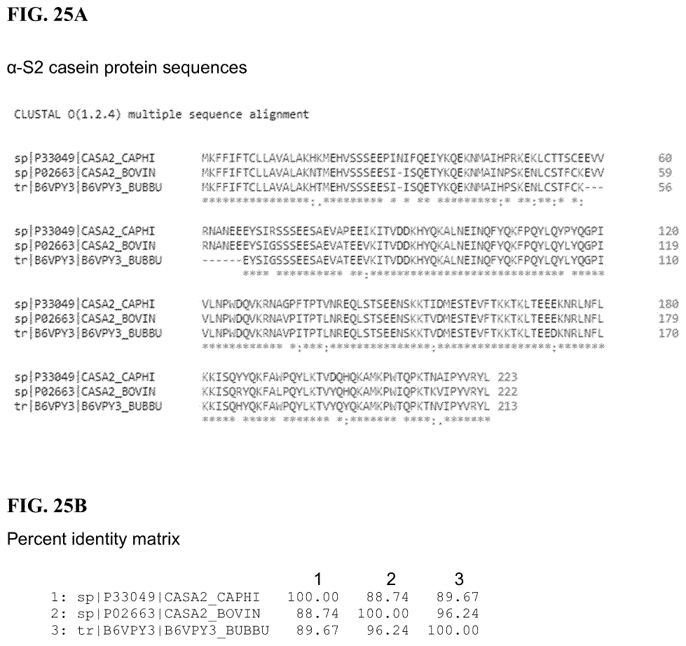

In some embodiments, the present disclosure provides sequence information of four types of casein protein (α-S1, α-S2, β-, κ-) from bovine (Bos taurus), human, goat (Capra hircus) and water buffalo (Bubalus bubalis). In some embodiments, α-S1, α-S2, β-, κ-casein protein sequences from bovine, human, goat, and water buffalo, as presented in FIG. 22A-25B, can be codon-optimized for expressing human, goat, and water buffalo casein proteins in plants disclosed in the present disclosure. Casein proteins from other species are well known in the art. See Barlowska, J., Szwajkowska, M., Litwiñczuk, Z., & Król, J. (2011). Nutritional value and technological suitability of milk from various animal species used for dairy production. Comprehensive reviews in food science and food safety, 10(6), 291-302, which is incorporated by reference in its entirety.

In some embodiments, the disclosure teaches κ-casein protein sequence that is generated from codon-optimized SEQ ID NO:1. In a particular embodiment, the κ-casein protein comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:5 is provided. In some embodiments, the κ-casein protein has the amino acid sequence of SEQ ID NO:5.

In some embodiments, the disclosure teaches κ-casein protein sequence without signal peptide that is generated from codon-optimized SEQ ID NO:2. In a particular embodiment, the κ-casein protein without signal peptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:6 is provided. In some embodiments, the κ-casein protein without signal peptide has the amino acid sequence of SEQ ID NO:6.

In other embodiments, the disclosure teaches β-casein protein sequence that is generated from codon-optimized SEQ ID NO:3. In a particular embodiment, the β-casein protein comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:7 is provided. In some embodiments, the β-casein protein has the amino acid sequence of SEQ ID NO:7.

In other embodiments, the disclosure teaches β-casein protein sequence without signal peptide that is generated from codon-optimized SEQ ID NO:4. In a particular embodiment, the β-casein protein without signal peptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:8 is provided. In some embodiments, the β-casein protein without signal peptide has the amino acid sequence of SEQ ID NO:8.

In other embodiments, the disclosure teaches α-S1 casein protein sequences that is generated from codon-optimized SEQ ID NO:9. In a particular embodiment, the α-S1 casein protein comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:11 is provided. In some embodiments, the α-S1 casein protein has the amino acid sequence of SEQ ID NO:11.

In other embodiments, the disclosure teaches α-S2 casein protein sequences that is generated from codon-optimized SEQ ID NO:10. In a particular embodiment, the α-S2 casein protein comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:12 is provided. In some embodiments, the α-S2 casein protein has the amino acid sequence of SEQ ID NO:12.

In other embodiments, the disclosure teaches α-lactalbumin protein sequence that is generated from codon-optimized SEQ ID NO:19. In a particular embodiment, the α-lactalbumin protein comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:22 is provided. In some embodiments, the α-lactalbumin protein has the amino acid sequence of SEQ ID NO:22.

In other embodiments, the disclosure teaches β-lactoglobulin protein sequences that is generated from codon-optimized SEQ ID NO:20. In a particular embodiment, the β-lactoglobulin protein comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:23 is provided. In some embodiments, the β-lactoglobulin casein protein has the amino acid sequence of SEQ ID NO:23.

In other embodiments, the disclosure teaches lysozyme protein sequences that is generated from codon-optimized SEQ ID NO:21. In a particular embodiment, the lysozyme casein protein comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO:24 is provided. In some embodiments, the α-S2 casein protein has the amino acid sequence of SEQ ID NO:24.

### Production of Transgenic Plants Expressing Bovine Milk Proteins

For producing transgenic plant stably expressing a protein of interest, plant cells or tissues are transformed with expression constructs (heterologous nucleic acid constructs, e.g., vectors/plasmids into which the gene of interest has been inserted) using various transformation techniques. It is preferred to use the vectors/plasmids in which foreign DNA sequences would be stably integrated into the host genome. In order to enhance plant gene expression, effective introduction of vectors/plasmids is an important aspect of the disclosure.

Integration of expression constructs into the host plant genome is preferably permanent so that the introduced expression constructs are inherited onto next plant generations. The skilled person in the art will recognize that a wide variety of transformation techniques exist in the art, and new techniques are continually becoming available.

Any technique that is suitable for the target host plant may be employed within the scope of the present disclosure. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. The skilled artisan can refer to the literature for details and select suitable techniques for use in the methods of the present disclosure. The methods for plant transformation practiced in the present disclosure are given.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety. In some embodiments, the present disclosure teaches the use of a variety of different transformation methods including, but not limited to, electroporation; microinjection; microprojectile bombardment, also known as particle acceleration or biolistic bombardment; viral-mediated transformation; and Agrobacterium-mediated transformation.

Agrobacterium tumefaciens is a naturally occurring bacterium that is capable of inserting its DNA (genetic information) into plants, resulting in a type of injury to the plant known as crown gall. Most species of plants can now be transformed using this method, including cucurbitaceous species.

The DNA constructs used for transformation in the methods of present disclosure may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for aminoglycoside adenyltransferase (aadA) conferring resistance to spectinomycin or streptomycin (e.g. U.S. Pat. No. 5,217,902; or Sandvang, 1999). For plant transformation, the host bacterial strain is often Agrobacterium tumefaciens ABI, C58, LBA4404, EHA101, or EHA105 carrying a plasmid having a transfer function for the expression unit. Other strains known to those skilled in the art of plant transformation can function in the present disclosure.

Bacterially-mediated gene delivery (e.g. Agrobacterium-mediated; U.S. Pat. Nos. 5,563,055; 5,591,616; 5,693,512; 5,824,877; 5,981,840) can be made into cells in the living meristem of an embryo excised from a seed (e.g. U.S. Pat. No. 6,384,301), and the meristematic region may be cultured in the presence of a selection agent such as spectinomycin. The result of this step is the termination or at least growth retardation of most of the cells into which the foreign genetic construction has not been delivered with the simultaneous formation of shoots, which arise from a single transformed meristematic cell, or small cluster of cells including transformed meristematic cells. In some embodiments, the meristem can be cultivated in the presence of spectinomycin, streptomycin or other selective agent, tolerance to which is encoded by the aadA gene. Examples of various selectable markers and genes providing resistance against them are disclosed in Miki and McHugh (2004) Journal of Biotechnology 107(3):193-232.

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The Biolistic® Gene Gun was invented in 1983-1984 at Cornell University by John Sanford, Edward Wolf, and Nelson Allen. It and its registered trademark are now owned by E. I. du Pont de Nemours and Company. Most species of plants have been transformed using this method.

The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1.

Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present disclosure. See, for example, U.S. Pat. No. 7,250,554, which utilizes P-DNA instead of T-DNA in the transformation vector.

A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988). Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. Nos. 5,204,253, 5,015,580). A third direct method uses fibrous forms of metal or ceramic consisting of sharp, porous or hollow needle-like projections that literally impale the cells, and also the nuclear envelope of cells. Both silicon carbide and aluminium borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. However, the efficiencies of each of these indirect or direct methods in introducing foreign DNA into plant cells are invariably extremely low, making it necessary to use some method for selection of only those cells that have been transformed, and further, allowing growth and regeneration into plants of only those cells that have been transformed. For efficient plant transformation, a selection method must be employed such that whole plants are regenerated from a single transformed cell and every cell of the transformed plant carries the DNA of interest. These methods can employ positive selection, whereby a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of non-transformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. Nos. 5,034,322, 6,174,724 and 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet 79: 625-631(1990), U.S. Pat. Nos. 4,795,855, 5,378,824 and 6,107,549). In addition, the dihydrofolate reductase (dhfr) gene, which confers resistance to the anticancer agent methotrexate, has been used for selection (Bourouis et al., EMBO J. 2(7): 1099-1104 (1983).

Non-limiting examples of binary vectors suitable for soybean species transformation and transformation methods are described by Yi et al. 2006 (Transformation of multiple soybean cultivars by infecting cotyledonary-node with Agrobacterium tumefaciens, African Journal of Biotechnology Vol. 5 (20), pp. 1989-1993, 16 Oct. 2006), Paz et al., 2004 (Assessment of conditions affecting Agrobacterium-mediated soybean transformation using the cotyledonary node explant, Euphytica 136: 167-179, 2004), U.S. Pat. Nos. 5,376,543, 5,416,011, 5,968,830, and 5,569,834, or by similar experimental procedures well known to those skilled in the art. Soybean plants can be transformed by using any method described in the above references.

The expression control elements used to regulate the expression of a given protein can either be the expression control element that is normally found associated with the coding sequence (homologous expression element) or can be a heterologous expression control element. A variety of homologous and heterologous expression control elements are known in the art and can readily be used to make expression units for use in the present disclosure. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Lastly, plant promoters such as prolifera promoter, fruit specific promoters, Ap3 promoter, heat shock promoters, seed specific promoters, etc. can also be used.

Either a gamete-specific promoter, a constitutive promoter (such as the CaMV or Nos promoter), an organ-specific promoter (such as the E8 promoter from tomato), or an inducible promoter is typically ligated to the protein or antisense encoding region using standard techniques known in the art. The expression unit may be further optimized by employing supplemental elements such as transcription terminators and/or enhancer elements.

Thus, for expression in plants, the expression units will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a pre-existing vector.

In the construction of heterologous promoter/structural gene or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835-846 (1984)) or the nopaline synthase signal (Depicker et al., Mol. and Appl. Genet. 1:561-573 (1982)). The resulting expression unit is ligated into or otherwise constructed to be included in a vector that is appropriate for higher plant transformation. One or more expression units may be included in the same vector. The vector will typically contain a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. Replication sequences, of bacterial or viral origin, are generally also included to allow the vector to be cloned in a bacterial or phage host; preferably a broad host range for prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector, as is known in the art. For instance, in the case of Agrobacterium transformations, T-DNA sequences will also be included for subsequent transfer to plant chromosomes.

To introduce a desired gene or set of genes by conventional methods requires a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as improved fatty acid composition, and to introduce these genes into already useful varieties of plants. Once the foreign genes have been introduced into a plant, that plant can then be used in conventional plant breeding schemes (e.g., pedigree breeding, single-seed-descent breeding schemes, reciprocal recurrent selection) to produce progeny which also contain the gene of interest.

Genes can be introduced in a site directed fashion using homologous recombination. Homologous recombination permits site-specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

### Host Plants for Transformation

The host plants used for transformation in the methods of the present disclosure include dicotyledonous and monocotyledonous plants. In some embodiments, the host plants used in the methods of the present disclosure are derived from dicots, including Arabidopsis, tobacco, tomato, potato, sweet potato, cassava, legumes including alfalfa, lima beans, pea, chick pea, soybean, carrot, strawberry, lettuce, oak, maple, walnut, rose, mint, squash, daisy, and cactus. Also another monocot host plant is selected from the group consisting of turf grass, maize (corn), rice, oat, wheat, barley, sorghum, orchid, iris, lily, onion, palm, and duckweed.

In order to produce transgenic plants that express bovine milk proteins, cells or tissues derived from the host plants are transformed with a recombinant DNA construct comprising the coding sequence for a bovine milk protein. The transformed plant cells or tissues express the coding sequence for the bovine milk protein, which is such a result of the successful integration of the heterologous nucleic acid construct. The appropriate selection agent in medium is used to identify and select the transformed plant cells or tissues that express the nucleic acid sequence encoding the bovine milk protein. Then, whole plants are regenerated from the selected plant cells or tissues that stably express the bovine milk protein from the heterologous nucleic acid sequences. Techniques for regenerating whole plants from transformed plant cells or tissues are well known in the art. Transgenic plant lines generated in the methods of the present disclosure can be maintained by genetic crosses using conventional plant breeding techniques.

In some embodiments, the present disclosure teaches methods of producing a transgenic plant, said methods comprising the steps of: (a) introducing at least one expression cassette capable of expressing a bovine milk protein into a plant, a part thereof, or a cell thereof; (b) obtaining the transgenic plant, the part thereof, or the cell thereof, which stably expresses the bovine milk protein; (c) cultivating the transgenic plant, the part thereof, or the cell thereof and (d) harvesting the transgenic plant, the part thereof, or the cell thereof. In some embodiments, the transgenic plant is a dicot plant selected from the group consisting of soybean, lima bean, Arabidopsis, and tobacco. In yet some embodiments, the transgenic plant is a monocot plant, such as rice and duckweed.

In some embodiments, the present disclosure teaches methods of producing a transgenic monocot plant, said methods comprising the steps of: (a) introducing at least one expression cassette capable of expressing a bovine milk protein into a monocot plant, a part thereof, or a cell thereof; (b) obtaining the transgenic monocot plant, the part thereof, or the cell thereof, which stably expresses the bovine milk protein; (c) cultivating the transgenic monocot plant, the part thereof, or the cell thereof; and (d) harvesting the transgenic monocot plant, the part thereof, or the cell thereof. In such embodiments the transgenic monocot plant is selected from the group consisting of turf grass, maize (corn), rice, oat, wheat, barley, sorghum, orchid, iris, lily, onion, palm, and duckweed. In yet some embodiments, the transgenic plant is a monocot plant, such as rice and duckweed.

In some embodiments, the present disclosure teaches methods of producing a transgenic dicot plant, said methods comprising the steps of: (a) introducing at least one expression cassette capable of expressing a bovine milk protein into a dicot plant, a part thereof, or a cell thereof (b) obtaining the transgenic dicot plant, the part thereof, or the cell thereof, which stably expresses the bovine milk protein; (c) cultivating the transgenic dicot plant, the part thereof, or the cell thereof; and (d) harvesting the transgenic dicot plant, the part thereof, or the cell thereof. In such embodiments, the transgenic dicot plant is selected from the group consisting of Arabidopsis, tobacco, tomato, potato, sweet potato, cassava, legumes including alfalfa, lima bean, pea, chick pea, soybean, carrot, strawberry, lettuce, oak, maple, walnut, rose, mint, squash, daisy, and cactus. In other embodiments, the transgenic dicot plant is selected from the group consisting of soybean, lima bean, Arabidopsis, and tobacco.

The present invention provides methods of producing a transgenic dicot or monocot plant containing the recombinant DNA constructs for milk protein expression. Such methods comprise utilizing the dicot or monocot plants comprising the chimeric genes as described herein.

The present invention also provides methods of breeding a transgenic dicot or monocot plant containing the recombinant DNA constructs for milk protein expression. In one embodiment, such methods comprise: i) making a cross between the dicot or monocot plant with nucleic acid sequences coding for bovine milk protein and/or fragments and variations thereof as described above to a second dicot or monocot plant to make F1 plants; ii) backcrossing said F1 plants to said second dicot or monocot plant, respectively; iii) repeating backcrossing step until said nucleic acid sequences are integrated into the genome of said second tomato or other plant species, respectively. Optionally, such method can be facilitated by molecular markers. In such embodiments the transgenic monocot plant is selected from the group consisting of turf grass, maize (corn), rice, oat, wheat, barley, sorghum, orchid, iris, lily, onion, palm, and duckweed. In yet some embodiments, the transgenic plant is a monocot plant, such as rice and duckweed. In such embodiments, the transgenic dicot plant is selected from the group consisting of Arabidopsis, tobacco, tomato, potato, sweet potato, cassava, legumes including alfalfa, lima bean, pea, chick pea, soybean, carrot, strawberry, lettuce, oak, maple, walnut, rose, mint, squash, daisy, and cactus. In other embodiments, the transgenic dicot plant is selected from the group consisting of soybean, lima bean, Arabidopsis, and tobacco.

### Detecting Expression of Recombinant Bovine Milk Protein

Transiently-transformed plant tissues or stably-transformed plant plants are screened for the expression of the recombinant bovine milk protein that may be confirmed using standard analytical techniques such as 1) antibody-dependent methods; enzyme-linked immunosorbent assay (ELISA), protein immunoprecipitation, immunoelectrophoresis, western blot, and protein immunostaining, together with assays for a biological activity specific to the particular protein being expressed, 2) spectrometry methods; high-performance liquid chromatography (HPLC), and liquid chromatography-mass spectrometry (LC/MS), and 3) PCR methods that detect expression level of recombinant transcripts, which can be an indicator of protein expression and abundance.

Examples of bovine milk proteins include α-S1 casein, α-S2 casein, β-casein, κ-casein, α-lactalbumin, β-lactoglobulin, serum albumin, lactoferrin, lysozyme, lactoperoxidase, immunoglobulin-A, and lipase. Recombinant DNA sequences in an expression vector/cassette in the present disclosure constructs may include genomic and cDNA sequences encoding bovine milk proteins as well as codon-optimized polynucleotide sequences encoding bovine milk proteins. When a recombinant DNA construct containing a bovine milk protein-coding sequence are used for stable transformation, the bovine milk protein-coding sequence of the recombinant DNA construct is preferably integrated in a random manner into the genome of the host plants used for transgenesis. Such random integration results in a transgenic plant will generally be located at a random position in the genome of the host plants.

Producing Bovine Milk Protein from Transgenic Plants

The transgenic plants of the present disclosure described herein contain milk proteins of mammalian origin in whole plants, preferably, in tissues and/or organs usually feasible as nutritionally enhanced foods. The contents of bovine milk proteins in the transgenic plants are not limited to specific ranges as far as they exhibit desirable amounts in transgenic plants. Protein content in the transgenic plants is about 1% or higher, 2% or higher, 3% or higher, 4% or higher, 5% or higher, 6% or higher, 7% or higher, 8% or higher, 9% or higher, preferably, 10% or higher, 20% or higher, 30% or higher, 40% or higher, 50% or higher, per the total protein weight of the soluble protein extractable from the whole plant and/or plant tissues/organs. Also, protein content in the transgenic plants are about 0.1 ng or higher, 1 ng or higher, 10 ng or higher, preferably 100 ng (=0.1 ug) or higher, 1 μg or higher, per one kilogram by fresh weight, while the values may be different based on the plant species or methods of transformation.

The present disclosure provides transgenic plants as a whole as they are harvested or plant parts as the form of isolated tissues of leaves, stems, roots, fruits, peels, buds, seeds, petals, other edible tissues or even inedible tissues. Depending on the need, the transgenic plants can be further processed by cutting, peeling, pulverizing, squeezing, extracting, or any other step into the form of cut vegetables, cut fruits, powders, juice, extracts, etc. Such methods of processing plants or plant parts are well known to those having ordinary skill in the art.

The present disclosure also provides the methods of producing a bovine milk protein from the transgenic plants. To produce the bovine milk protein, the total proteins, including soluble and insoluble proteins, are isolated and extracted from the transgenic plants as a whole and/or the form of isolated tissues of leaves, stems, roots, fruits, peels, buds, seeds, petals, other edible tissues, or even inedible tissues. The total proteins can be separated into soluble and insoluble proteins depending on the purpose of purifying the bovine milk protein. According to the features of the bovine milk protein solubility, the bovine milk protein of interest may be further purified. The methods of processing plants or plant parts are well known to those of ordinary skill in the art. See, for example, U.S. Pat. Nos. 5,891,433 and 6,455,759; U.S. Patent Application Publication Nos. 2002/0002714 and 2016/0220622; and International Patent Application Publication Nos. WO/1999/024592 and WO/2016197584, and each of which is expressly incorporated herein by reference in their entirety.

In some embodiments, the present disclosure teaches methods of producing a bovine milk protein from a transgenic plant, said methods comprising the steps of: (a) extracting the bovine milk protein from the transgenic plant, the part thereof, or the cell thereof; and (b) purifying the bovine milk protein from the transgenic plant, the part thereof, or the cell thereof. In some embodiments, the transgenic plant is a dicot plant selected from the group consisting of soybean, lima bean, Arabidopsis, and tobacco. In yet some embodiments, the transgenic plant is a monocot plant, such as rice and duckweed.

In other embodiments, the present disclosure teaches methods of producing a bovine milk protein from a transgenic monocot plant, said methods comprising the steps of: (a) extracting the bovine milk protein from the transgenic monocot plant, the part thereof, or the cell thereof; and (b) purifying the bovine milk protein from the transgenic monocot plant, the part thereof, or the cell thereof. In such embodiments the transgenic monocot plant is selected from the group consisting of turf grass, maize (corn), rice, oat, wheat, barley, sorghum, orchid, iris, lily, onion, palm, and duckweed. In yet some embodiments, the transgenic plant is a monocot plant, such as rice and duckweed.

In some embodiments, the present disclosure teaches a method of producing a bovine milk protein from a transgenic dicot plant, said method comprising the steps of: (a) extracting the bovine milk protein from the transgenic dicot plant, the part thereof, or the cell thereof; and (b) purifying the bovine milk protein from the transgenic dicot plant, the part thereof, or the cell thereof. In such embodiments, the transgenic dicot plant is selected from the group consisting of Arabidopsis, tobacco, tomato, potato, sweet potato, cassava, legumes including alfalfa, lima beans, pea, chick pea, soybean, carrot, strawberry, lettuce, oak, maple, walnut, rose, mint, squash, daisy, and cactus. In embodiments, the transgenic dicot plant is selected from the group consisting of soybean, Arabidopsis, and tobacco.

### Producing Milk Proteins in Embryos

Biolistically transformed somatic embryos have been used to quickly assess the effect of a transgenic construct on the seed protein, as in Herman et al (2003) Plant Physiology 132(1):36-43. Soybean somatic embryos cultured in Soybean Histodifferentiation and Maturation (SHaM) medium were examined for their suitability as a model system for developing an understanding of assimilate partitioning and metabolic control points for protein and oil biosynthesis in soybean seed (Truong (2013) J Exp Bot. 64(10): 2985-2995). A modified soybean histodifferentiation and maturation (SHaM) medium produces differentiated embryos that are similar in protein and lipid composition to seed, expediting protein analysis. The similarity between SHaM matured embryos and seed was demonstrated in Nishizawa and Ishimoto (2009) Plant Biotechnology 26(5):543-550, by showing that the total protein profile shifts over the maturation period, closely resembling dry seed by 25 days, including the accumulation of important seed-storage proteins. Soybean somatic embryos therefore appear to be a suitable experimental model with which to study the synthesis of seed components.

Also, the presence of protein storage body specific proteins was confirmed by electron microscopy. Herman et al. (2014; Frontiers in Plant Science 5:437) showed that the cotyledon, rather than the axis, of mature suspension embryos express seed promoted transgenes, like stable transformants do in seed. Pierce et al (2015, PloS one 10(9), e0138196) demonstrated this dramatically when carotenoid metabolites were produced in both mature embryos and transgenic seed, changing the color of the tissue to dark orange, but other plant structures were left unaffected when transgenes were under the control of the seed specific lectin promoter. Oleic acid composition of SHaM matured embryos was demonstrated, but T1 seed was also used as outlined in U.S. Pat. No. 8,927,809. SHaM embryos can be a system for the evaluation of transgenic approaches to improve soybean quality. Somatic mature embryo model systems are well known to those of ordinary skill in the art. See, for example, Schmidt (2005) Plant Cell Reports 24:383-391; Truong, Q. et al, Journal of Experimental Botany (2013) 64(1), 2985-2995; Herman, E. M., Frontiers in Plant Science (2014) 5:437; Pierce et al (2015, PloS one 10(9), e0138196); Nishizawa, K. and Ishimoto, M., Plant Biotechnology (2009) 26(5):543-550 and U.S. Pat. No. 8,927,809; each of which is expressly incorporated herein by reference in their entirety.

SHaM embryos show greater developmental uniformity, have compositions that are more seed like (Schmidt (2005) Plant Cell Reports 24:383-391), and have proven to be an excellent system for testing genes that lead to oil content increases in mature seed (Meyer et al., 2012, U.S. Pat. No. 8,143,473). SHaM embryos are now a proven system for the evaluation of transgenic approaches to improve soybean quality.

Somatic embryos are the target tissue for one commonly used method of soybean genetic transformation (Finer and McMullen, 1991). Somatic embryos can provide a preview of the ultimate composition of mature soybean seed, within 10 weeks of the initial transformation (Kinney (1996) Journal of Food Lipids 3 (4): 273-292).

In some embodiments, the present disclosure teaches soybean somatic embryo model systems such as mature embryos, somatic embryos, SHaM embryos, a modified Sham embryo, and/or embryogenic callus. In one embodiment, the present disclosure teaches matured embryos for producing milk proteins disclosed in the present disclosure. In another embodiment, the present disclosure teaches somatic embryos for producing milk proteins disclosed in the present disclosure. In further another embodiment, the present disclosure teaches SHaM embryos for producing milk proteins disclosed in the present disclosure. In yet another embodiment, the present disclosure teaches embryogenic callus tissue for producing milk proteins disclosed in the present disclosure.

### Post-Translational Modification of Proteins

Protein post-translational modification (PTM) increases the functional diversity of the proteome by the covalent addition of functional groups or proteins, proteolytic cleavage of regulatory subunits or degradation of entire proteins. These modifications include phosphorylation, glycosylation, ubiquitination, nitrosylation, methylation, acetylation, lipidation and proteolysis and influence almost all aspects of normal cell biology and pathogenesis. The proteins of the disclosure may undergo one or more post-translational modifications.

Within the last few decades, scientists have discovered that the human proteome is vastly more complex than the human genome. While it is estimated that the human genome comprises between 20,000 and 25,000 genes, the total number of proteins in the human proteome is estimated at over 1 million. See, e.g., International Human Genome Sequencing Consortium (2004) “Finishing the euchromatic sequence of the human genome.” Nature. 431, 931-45; and Jensen O. N. (2004) “Modification-specific proteomics: Characterization of post-translational modifications by mass spectrometry.” Curr Opin Chem Biol. 8, 33-41.

These estimations demonstrate that single genes encode multiple proteins. Genomic recombination, transcription initiation at alternative promoters, differential transcription termination, and alternative splicing of the transcript are mechanisms that generate different mRNA transcripts from a single gene. See, Ayoubi T. A. and Van De Ven W. J. (1996) “Regulation of gene expression by alternative promoters.” FASEB J. 10, 453-60.

The increase in complexity from the level of the genome to the proteome is further facilitated by protein post-translational modifications (PTMs). PTMs are chemical modifications that play a key role in functional proteomics, because they regulate activity, localization and interaction with other cellular molecules such as proteins, nucleic acids, lipids, and cofactors.

Post-translational modifications are key mechanisms to increase proteomic diversity. While the genome comprises 20-25,000 genes, the proteome is estimated to encompass over 1 million proteins. Changes at the transcriptional and mRNA levels increase the size of the transcriptome relative to the genome, and the myriad of different post-translational modifications exponentially increases the complexity of the proteome relative to both the transcriptome and genome.

Additionally, the human proteome is dynamic and changes in response to a legion of stimuli, and post-translational modifications are commonly employed to regulate cellular activity. PTMs occur at distinct amino acid side chains or peptide linkages and are most often mediated by enzymatic activity. Indeed, it is estimated that 5% of the proteome comprises enzymes that perform more than 200 types of post-translational modifications. See, Walsh C. (2006) “Posttranslational modification of proteins: Expanding nature's inventory.” Englewood, Colo.: Roberts and Co. Publishers. xxi, 490 p.p. These enzymes include kinases, phosphatases, transferases and ligases, which add or remove functional groups, proteins, lipids or sugars to or from amino acid side chains, and proteases, which cleave peptide bonds to remove specific sequences or regulatory subunits. Many proteins can also modify themselves using autocatalytic domains, such as autokinase and autoprotolytic domains.

Post-translational modification can occur at any step in the life cycle of a protein. For example, many proteins are modified shortly after translation is completed to mediate proper protein folding or stability or to direct the nascent protein to distinct cellular compartments (e.g., nucleus, membrane). Other modifications occur after folding and localization are completed to activate or inactivate catalytic activity or to otherwise influence the biological activity of the protein. Proteins are also covalently linked to tags that target a protein for degradation. Besides single modifications, proteins are often modified through a combination of post-translational cleavage and the addition of functional groups through a step-wise mechanism of protein maturation or activation.

Protein PTMs can also be reversible depending on the nature of the modification. For example, kinases phosphorylate proteins at specific amino acid side chains, which is a common method of catalytic activation or inactivation. Conversely, phosphatases hydrolyze the phosphate group to remove it from the protein and reverse the biological activity. Proteolytic cleavage of peptide bonds is a thermodynamically favorable reaction and therefore permanently removes peptide sequences or regulatory domains.

As noted above, there are a large number of different PTMs that are possible with respect to the disclosed proteins. A few of the common PTMs are discussed below.

i. Phosphorylation

Reversible protein phosphorylation, principally on serine, threonine or tyrosine residues, is one of the most important and well-studied post-translational modifications. Phosphorylation plays critical roles in the regulation of many cellular processes including cell cycle, growth, apoptosis and signal transduction pathways

ii. Glycosylation

Protein glycosylation is acknowledged as one of the major post-translational modifications, with significant effects on protein folding, conformation, distribution, stability and activity. Glycosylation encompasses a diverse selection of sugar-moiety additions to proteins that ranges from simple monosaccharide modifications of nuclear transcription factors to highly complex branched polysaccharide changes of cell surface receptors. Carbohydrates in the form of aspargine-linked (N-linked) or serine/threonine-linked (O-linked) oligosaccharides are major structural components of many cell surface and secreted proteins.

iii. Ubiquitination

Ubiquitin is an 8-kDa polypeptide consisting of 76 amino acids that is appended to the Îμ-NH2 of lysine in target proteins via the C-terminal glycine of ubiquitin. Following an initial monoubiquitination event, the formation of a ubiquitin polymer may occur, and polyubiquitinated proteins are then recognized by the 26S proteasome that catalyzes the degradation of the ubiquitinated protein and the recycling of ubiquitin.

iv. S-Nitrosylation

Nitric oxide (NO) is produced by three isoforms of nitric oxide synthase (NOS) and is a chemical messenger that reacts with free cysteine residues to form S-nitrothiols (SNOs). S-nitrosylation is a critical PTM used by cells to stabilize proteins, regulate gene expression and provide NO donors, and the generation, localization, activation and catabolism of SNOs are tightly regulated.

S-nitrosylation is a reversible reaction, and SNOs have a short half life in the cytoplasm because of the host of reducing enzymes, including glutathione (GSH) and thioredoxin, that denitrosylate proteins. Therefore, SNOs are often stored in membranes, vesicles, the interstitial space and lipophilic protein folds to protect them from denitrosylation. See, Gaston B. M. et al. (2003) “S-nitrosylation signaling in cell biology.” Mol Interv. 3, 253-63. For example, caspases, which mediate apoptosis, are stored in the mitochondrial intermembrane space as SNOs. In response to extra- or intracellular cues, the caspases are released into the cytoplasm, and the highly reducing environment rapidly denitrosylates the proteins, resulting in caspase activation and the induction of apoptosis.

S-nitrosylation is not a random event, and only specific cysteine residues are S-nitrosylated. Because proteins may contain multiple cysteines and due to the labile nature of SNOs, S-nitrosylated cysteines can be difficult to detect and distinguish from non-S-nitrosylated amino acids. The biotin switch assay, developed by Jaffrey et al., is a common method of detecting SNOs, and the steps of the assay are listed below. See, Jaffrey S. R. and Snyder S. H. (2001) “The biotin switch method for the detection of S-nitrosylated proteins.” Sci STKE. 2001, p 11. All free cysteines are blocked.→All remaining cysteines (presumably only those that are denitrosylated) are denitrosylated.→The now-free thiol groups are then biotinylated. Biotinylated proteins are detected by SDS-PAGE and Western blot analysis or mass spectrometry. See, Han P. and Chen C. (2008) “Detergent-free biotin switch combined with liquid chromatography/tandem mass spectrometry in the analysis of S-nitrosylated proteins.” Rapid Commun Mass Spectrom. 22, 1137-45.

v. Methylation

The transfer of one-carbon methyl groups to nitrogen or oxygen (N- and O-methylation, respectively) to amino acid side chains increases the hydrophobicity of the protein and can neutralize a negative amino acid charge when bound to carboxylic acids. Methylation is mediated by methyltransferases, and S-adenosyl methionine (SAM) is the primary methyl group donor.

Methylation occurs so often that SAM has been suggested to be the most-used substrate in enzymatic reactions after ATP. Additionally, while N-methylation is irreversible, O-methylation is potentially reversible. Methylation is a well-known mechanism of epigenetic regulation, as histone methylation and demethylation influences the availability of DNA for transcription. Amino acid residues can be conjugated to a single methyl group or multiple methyl groups to increase the effects of modification.

vi. N-Acetylation

N-acetylation, or the transfer of an acetyl group to nitrogen, occurs in almost all eukaryotic proteins through both irreversible and reversible mechanisms. N-terminal acetylation requires the cleavage of the N-terminal methionine by methionine aminopeptidase (MAP) before replacing the amino acid with an acetyl group from acetyl-CoA by N-acetyltransferase (NAT) enzymes. This type of acetylation is co-translational, in that N-terminus is acetylated on growing polypeptide chains that are still attached to the ribosome. While 80-90% of eukaryotic proteins are acetylated in this manner, the exact biological significance is still unclear.

Acetylation at the ε-NH₂ of lysine (termed lysine acetylation) on histone N-termini is a common method of regulating gene transcription. Histone acetylation is a reversible event that reduces chromosomal condensation to promote transcription, and the acetylation of these lysine residues is regulated by transcription factors that contain histone acetyltransferase (HAT) activity. While transcription factors with HAT activity act as transcription co-activators, histone deacetylase (HDAC) enzymes are co-repressors that reverse the effects of acetylation by reducing the level of lysine acetylation and increasing chromosomal condensation.

Sirtuins (silent information regulator) are a group of NAD-dependent deacetylases that target histones. As their name implies, they maintain gene silencing by hypoacetylating histones and have been reported to aid in maintaining genomic stability. See, Imai S. et al. (2000) “Transcriptional silencing and longevity protein SIR2 is an NAD-dependent histone deacetylase.” Nature. 403, 795-800.

While acetylation was first detected in histones, cytoplasmic proteins have been reported to also be acetylated, and therefore acetylation seems to play a greater role in cell biology than simply transcriptional regulation. See, Glozak M. A. et al. (2005) “Acetylation and deacetylation of non-histone proteins.” Gene. 363, 15-23. Furthermore, crosstalk between acetylation and other post-translational modifications, including phosphorylation, ubiquitination and methylation, can modify the biological function of the acetylated protein. See, Yang X. J. and Seto E. (2008) “Lysine acetylation: Codified crosstalk with other posttranslational modifications.” Mol Cell. 31, 449-61.

Protein acetylation can be detected by chromosome immunoprecipitation (ChIP) using acetyllysine-specific antibodies or by mass spectrometry, where an increase in histone by 42 mass units represents a single acetylation.

vii. Lipidation

Lipidation is a method to target proteins to membranes in organelles (endoplasmic reticulum [ER], Golgi apparatus, mitochondria), vesicles (endosomes, lysosomes) and the plasma membrane. The four types of lipidation are: C-terminal glycosyl phosphatidylinositol (GPI) anchor; N-terminal myristoylation; S-myristoylation; and S-prenylation. Each type of modification gives proteins distinct membrane affinities, although all types of lipidation increase the hydrophobicity of a protein and thus its affinity for membranes. The different types of lipidation are also not mutually exclusive, in that two or more lipids can be attached to a given protein.

GPI anchors tether cell surface proteins to the plasma membrane. These hydrophobic moieties are prepared in the ER, where they are then added to the nascent protein en bloc. GPI-anchored proteins are often localized to cholesterol- and sphingolipid-rich lipid rafts, which act as signaling platforms on the plasma membrane. This type of modification is reversible, as the GPI anchor can be released from the protein by phosphoinositol-specific phospholipase C. Indeed, this lipase is used in the detection of GPI-anchored proteins to release GPI-anchored proteins from membranes for gel separation and analysis by mass spectrometry.

N-myristoylation is a method to give proteins a hydrophobic handle for membrane localization. The myristoyl group is a 14-carbon saturated fatty acid (C14), which gives the protein sufficient hydrophobicity and affinity for membranes, but not enough to permanently anchor the protein in the membrane. N-myristoylation can therefore act as a conformational localization switch, in which protein conformational changes influence the availability of the handle for membrane attachment. Because of this conditional localization, signal proteins that selectively localize to membrane, such as Src-family kinases, are N-myristoylated.

N-myristoylation is facilitated specifically by N-myristoyltransferase (NMT) and uses myristoyl-CoA as the substrate to attach the myristoyl group to the N-terminal glycine. Because methionine is the N-terminal amino acid of all eukaryotic proteins, this PTM requires methionine cleavage by the above-mentioned MAP prior to addition of the myristoyl group; this represents one example of multiple PTMs on a single protein.

S-palmitoylation adds a C16 palmitoyl group from palmitoyl-CoA to the thiolate side chain of cysteine residues via palmitoyl acyl transferases (PATs). Because of the longer hydrophobic group, this anchor can permanently anchor the protein to the membrane. This localization can be reversed, though, by thioesterases that break the link between the protein and the anchor; thus, S-palmitoylation is used as an on/off switch to regulate membrane localization. S-palmitoylation is often used to strengthen other types of lipidation, such as myristoylation or farnesylation. S-palmitoylated proteins also selectively concentrate at lipid rafts.

S-prenylation covalently adds a farnesyl (C15) or geranylgeranyl (C20) group to specific cysteine residues within 5 amino acids from the C-terminus via farnesyl transferase (FT) or geranylgeranyl transferases (GGT I and II). Unlike S-palmitoylation, S-prenylation is hydrolytically stable. Approximately 2% of all proteins are prenylated, including all members of the Ras superfamily. This group of molecular switches is farnesylated, geranylgeranylated or a combination of both. Additionally, these proteins have specific 4-amino acid motifs at the C-terminus that determine the type of prenylation at single or dual cysteines. Prenylation occurs in the ER and is often part of a stepwise process of PTMs that is followed by proteolytic cleavage by Rce1 and methylation by isoprenyl cysteine methyltransferase (ICMT).

viii. Proteolysis

Peptide bonds are indefinitely stable under physiological conditions, and therefore cells require some mechanism to break these bonds. Proteases comprise a family of enzymes that cleave the peptide bonds of proteins and are critical in antigen processing, apoptosis, surface protein shedding, and cell signaling.

The family of over 11,000 proteases varies in substrate specificity, mechanism of peptide cleavage, location in the cell and the length of activity. While this variation suggests a wide array of functionalities, proteases can generally be separated into groups based on the type of proteolysis. Degradative proteolysis is critical to remove unassembled protein subunits and misfolded proteins and to maintain protein concentrations at homeostatic concentrations by reducing a given protein to the level of small peptides and single amino acids. Proteases also play a biosynthetic role in cell biology that includes cleaving signal peptides from nascent proteins and activating zymogens, which are inactive enzyme precursors that require cleavage at specific sites for enzyme function. In this respect, proteases act as molecular switches to regulate enzyme activity.

Proteolysis is a thermodynamically favorable and irreversible reaction. Therefore, protease activity is tightly regulated to avoid uncontrolled proteolysis through temporal and/or spatial control mechanisms including regulation by cleavage in cis or trans and compartmentalization (e.g., proteasomes, lysosomes).

The diverse family of proteases can be classified by the site of action, such as aminopeptidases and carboxypeptidase, which cleave at the amino or carboxy terminus of a protein, respectively. Another type of classification is based on the active site groups of a given protease that are involved in proteolysis. Based on this classification strategy, greater than 90% of known proteases fall into one of four categories as follows: Serine proteases, Cysteine proteases, Aspartic acid proteases, and Zinc metalloproteases. However, Threonine protease, Glutamic protease, Asparagine peptide lyase are also known as other proteases.

In some embodiments, the present disclosure teaches proteolysis of milk proteins including, but not limited to α-S1 casein, α-S2 casein, β-casein, κ-casein, α-lactalbumin, β-lactoglobulin, serum albumin, lactoferrin, lysozyme, lactoperoxidase, immunoglobulin-A, and lipase. In some embodiments, the present disclosure teaches that the proteolysis of milk proteins results in formation of peptides and free amino acids. In other embodiments, the proteolysis of milk proteins is the proteolysis of casein proteins including α-S1 casein, α-S2 casein, β-casein, and κ-casein, which results in formation of peptides and free amino acids. In some embodiments, peptides and free amino acids, which are proteolyzed from the milk proteins are partly responsible for taste descriptors. In other embodiments, the present disclosure teaches proteolysis of the milk proteins produces proteolytic products of casein proteins such as casein-like peptides.

### Food Applications

Milk protein concentrates (MPCs) and milk protein isolates (MPIs) are high-quality proteins that are present in milk. MPCs and MPIs contain both casein and whey proteins in the similar ratio as milk, which are functionally active without being denatured. Specifically, MPCs and MPIs become important source of protein for nutritional and functional properties in various commercial applications because proteins in them are higher than whole or skim milk powder while lactose is lower (Patel et al, 2014).

Dependent on the protein content, MPCs can be utilized for nutrition-enhanced products. For example, lower-protein MPCs (42 to 50% protein content) are used as ingredients in cheese, yogurt, and soup products, while higher-protein MPCs (70% or higher protein content) are used as ingredients in beverages, medical foods, enteral foods, weight management products, powdered dietary supplements, sports nutrition products, and protein bar products.

In one aspect of the present disclosure, the transgenic plants or a part thereof can be used to produce important ingredients and components in foods to improve nutritional properties, but not limited to, such as processed cheese, cream cheese, fresh cheese, yogurt and fermented dairy products, frozen dairy products, desserts, baked goods, toppings, low-fat spreads, dairy-based dry mixes, soups, sauces, salad dressing, geriatric nutrition, ice cream, creamer, follow-up formula, baby formula, infant formula, milk, butter alternatives, growing up milks, low-lactose products and beverages, medical and clinical nutrition products, protein/nutrition bar applications, sports beverages, meal replacement beverages, and weight management food and beverages. In yet other embodiments, the transgenic plants or a part thereof can be processed into a powder form by grinding as a dietary supplement or baking powder. In some embodiments, the transgenic soybeans could be used for traditional soy food production, such as tofu, soy milk, miso, soy sauce, tempeh, natto, teriyaki, and meat alternatives, etc. In some embodiments, transgenic cereal plants, including maize (corn), rice, barley, wheat, oat, sorghum, millet, rye, triticale, fonio, buckwheat, quinoa, and chia, may produce bovine casein proteins and grains of the transgenic cereal plants may be ground into the form of powder for the purpose of dietary supplement or the use of a baking powder.

In one aspect of the present disclosure, the transgenic plants or a part thereof can be used to produce milk that contains at least 20% A2 beta-casein by weight of total beta-casein, at least 30% A2 beta-casein by weight of total beta-casein, at least 40% A2 beta-casein by weight of total beta-casein, at least 50% A2 beta-casein by weight of total beta-casein, at least 60% A2 beta-casein by weight of total beta-casein, at least 70% A2 beta-casein by weight of total beta-casein, at least 80% A2 beta-casein by weight of total beta-casein, at least 90% A2 beta-casein by weight of total beta-casein, at least 95% A2 beta-casein by weight of total beta-casein, and 100% A2 beta-casein by weight of total beta-casein.

In another aspect of the present disclosure, the transgenic plants or a part thereof comprising a recombinant DNA construction encoding specific variant of beta-casein including A1, A2, A3, B, C, D, E, F, H1, H2, I, and G using synthetic biology can be used to produce including, but not limited to milk, baby formula, infant formula, various dairy products such as cheese, cream cheese, fresh cheese, yogurt and fermented dairy products, frozen dairy products. In further embodiments, the transgenic plants or a part thereof comprising a recombinant DNA construction encoding specific variant of beta-casein including A2 beta-casein can be used to produce including, but not limited to milk, baby formula, infant formula, various dairy products such as cheese, cream cheese, fresh cheese, yogurt and fermented dairy products, frozen dairy products.

In another aspect of the present disclosure, the transgenic plants or a part thereof can be also used to produce important ingredients and components in foods to enhance functional properties, but not limited to, such as water binding, thickening, viscosity, emulsification, foaming and whipping, gelling/gelation, heat stability, and color/flavor development. The functional properties of bovine milk proteins extracted form transgenic plants and a part thereof can be applied for a variety of food products; for example, 1) water binding, thickening, and viscosity may be applied for soups, sauces, meat products, bakery products, confectionary, chocolate, yogurt, and cheese, 2) emulsification for soups, sauces, ice cream, confectionary, meat products, coffee whitener, 3) foaming and whipping for ice cream, desserts, and whipped toppings, 4) gelation (gelling) for cheese, yogurt, bakery, and confectionary, 5) heat stability for recombined milk, soups, sauces, and clinical nutrition, and 6) color/flavor development for chocolate and confectionary. The methods of manufacturing dairy substitutes, and compositions comprising animal-free milk fats and proteins for food application is well known to those of ordinary skill in the art. See, for example, International Patent Application Publication No. WO/2016/029193, which is expressly incorporated herein by reference in its entirety.

In other aspects, the bovine protein can be isolated, concentrated, and/or hydrolyzed from the transgenic plants to make protein isolate or protein concentrate or protein hydrolysate, which is used to make products described above according to the nutritional and functional properties.

### Non-Food Applications

Among milk proteins, casein proteins have preferably been used in the production of nonfood products for decades. The main protein in bovine milk, casein is based on four major components, α-S1 casein (38%), α-S2-casein (10%), β-casein (36%), and κ-casein (13%) and a minor constituent, γ-CN (3%). Each constituent varies in amino acid composition, molecular weight, isoelectric point and hydrophilicity (Kinsella et al., 1984, and Kinsella et al., 1989). Due to high amount of polar groups, solubility, molecular flexibility for intermolecular interactions, availability of chemical modification, casein can be used in several technical applications such as protective coating and foams, paper coating, adhesives or injection molding disposables. For example, casein can be used for 1) coating such as paint, ink, paper, packaging, leather finishing, textile coating, 2) adhesive such as a water-based glue, 3) plastic such as rigid or disposable plastic, film or foil in packaging application, and 4) surfactant like emulsifier or detergent.

In some aspects of the present disclosure, the bovine milk proteins, preferably, casein proteins, extracted and/or purified from the transgenic plants or a part thereof can be used to manufacture plastics in the form of bags, packaging material, buttons, buckles, and imitation ivory. In other aspects of the present disclosure, the bovine milk proteins, preferably, casein proteins, extracted and/or purified from the transgenic plants or a part thereof also can be used as adhesive for wood (i.e. plywood), coatings for paper and cardboard, synthetic fibers, and paints. Furthermore, the bovine milk proteins described herein can be applied for manufacturing emulsifier, detergent and/or tooth remineralization products.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will occur to those skilled in the art.

The following experiments demonstrate constructions of codon-optimized DNA sequences of milk proteins, methods of testing and producing the demonstrated proteins, and results from the experiments.

### Example 1. Construction of Expression Vectors for Plant Transformation for Transient and Stable Expression of Recombinant Milk Proteins

The soybean codon-optimized nucleic acid sequences coding for milk proteins were synthesized and cloned into a binary vector pCambia1305.1, respectively. The codon-optimized DNA sequences are listed in Table 2 as follows; i) OKC1 (Optimized Kappa Casein version 1) ii) OKC1-T (Optimized Kappa Casein Truncated version 1), iii) OBC1 (Optimized Beta Casein version 1), iv) OBC1-T (Optimized Beta Casein Truncated version 1), v) OS1C1 (Optimized alpha S1 Casein version 1), vi) OS2C1 (Optimized alpha S2 Casein version 1), vii) OLA1 (Optimized Alpha Lactalbumin version 1), viii) OLG1 (Optimized Beta Lactoglobulin 1), and ix) OLY1 (Optimized Lysozyme C version 1).

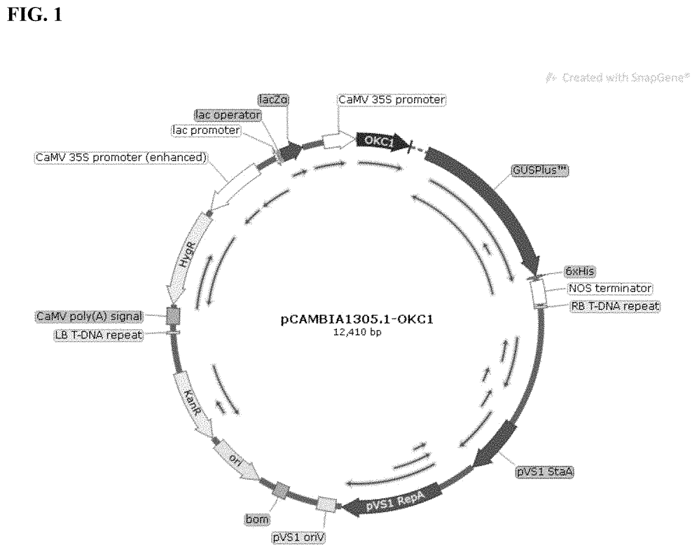

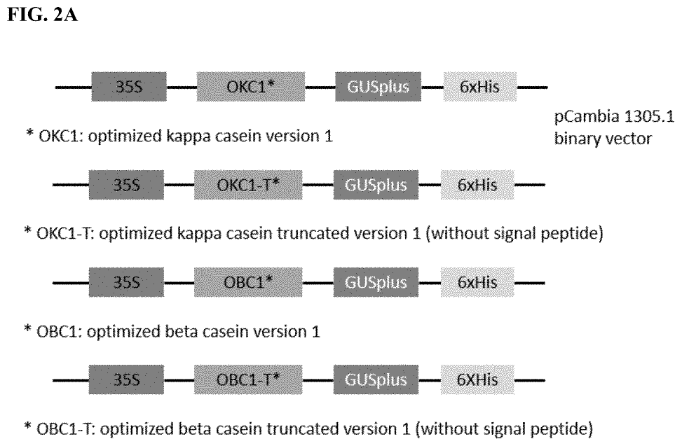

Each of the codon-optimized nucleic acid sequences encoding milk proteins was inserted between a CaMV 35S promoter and GUSPlus™ gene in a binary vector pCambia1305.1. For the insert ligation, two restriction enzymes, NcoI and BglII, were used for creating blunt ends in the pCambia1305.1 vector and the codon-optimized DNA insert. As an example, the pCambia1305.1 vector fused with OKC1 (Optimized Kappa Casein version 1) insert is illustrated in FIG. 1. Using the same experimental procedures various transgene constructs were generated for testing expression of milk proteins including α-S1 casein, α-S2 casein, β-casein, κ-casein, α-lactalbumin, β-lactoglobulin, and lysozyme. FIG. 2A-2B illustrates that an individual transgene is driven by 35S promoter and fused with GUSPlus™ and 6×His-tag, which is followed by Nos-terminator. Four constructs are illustrated in FIG. 2A with four distinct types of transgenes encoding milk proteins such as casein proteins. Each transgene is labeled as i) OKC1 (Optimized Kappa Casein version 1) ii) OKC1-T (Optimized Kappa Casein version 1-Truncated), iii) OBC1 (Optimized Beta Casein version 1), and OBC1-T (Optimized Beta Casein version 1-Truncated). The truncated version of transgenes do not have signal peptide sequence at their 5′ end. FIG. 2B illustrates three constructs were generated with three distinct types of transgenes encoding milk proteins such as whey proteins. Each transgene is labeled as i) OLA1 (Optimized Alpha Lactalbumin version 1), ii) OLG1 (Optimized Beta Lactoglobulin 1), and iii) OLY1 (Optimized Lysozyme C version 1). The protein expression can be detected visually by GUS staining assay and measured by western blot analysis using Anti-6×His tag antibody.

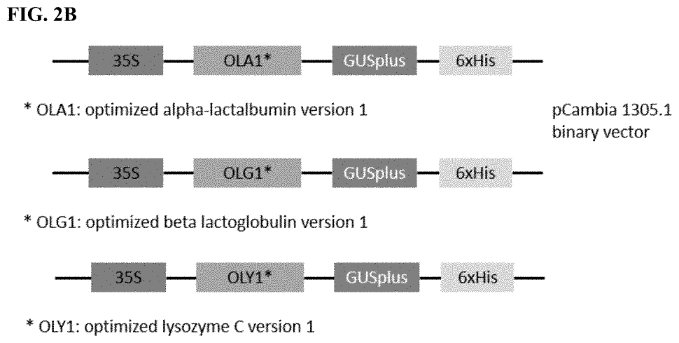

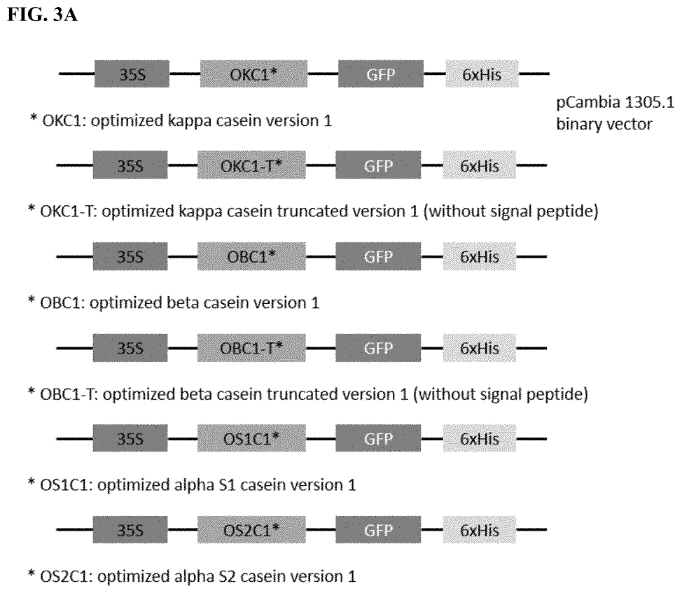

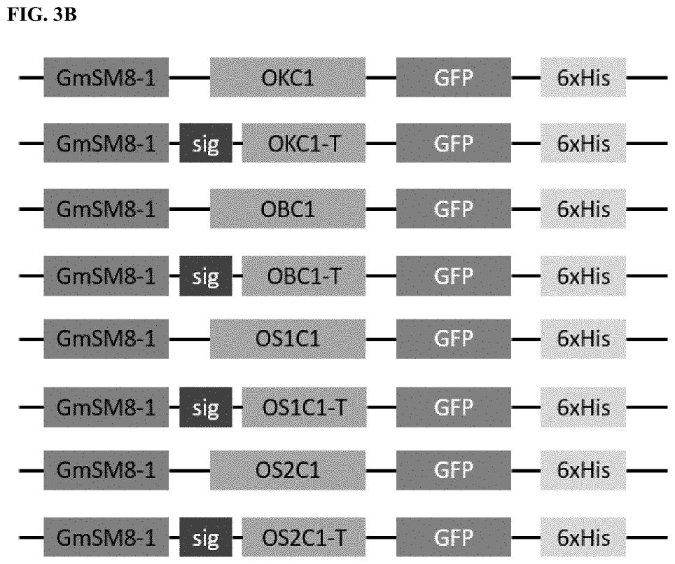

Furthermore, other transgene expression vectors were generated with a new selectable marker GFP gene. These three new sets of expression vectors have a new visible GFP marker instead of GUSPlus™ as illustrated in FIGS. 3A-3C. In these sets, three different types of promoters were utilized for driving expression of milk proteins in these expression vectors. First, a constitutive CaMV 35S promoter was adopted for expressing GFP-fused milk proteins including κ-casein, β-casein, α-S1 casein, and α-S2 casein. Six transgenes, disclosed in FIG. 3A and Table 2, were fused with GFP:6×His-tag (SEQ ID NO:14) in a forward direction, and then is followed by Nos-terminator for transcription termination. Six constructs were generated with six distinct types of transgenes encoding milk proteins. Each transgene is labeled as i) OKC1 (Optimized Kappa Casein version 1), ii) OKC1-T (Optimized Kappa Casein Truncated version 1), iii) OBC1 (Optimized Beta Casein version 1), iv) OBC1-T (Optimized Beta Casein Truncated version 1), v) OS1C1 (Optimized alpha S1 Casein version 1), vi) 052C1 (Optimized alpha S2 Casein version 1). The protein expression was detected visually by GFP expression under blue light (488 nm) and measured by western blot analysis using Anti-His tag antibody.

Second, a series of six constructs were generated out of eight expression cassettes illustrated in FIG. 3B. Eight distinct types of transgenes encoding milk proteins are listed as i) OKC1 (Optimized Kappa Casein version 1), ii) OKC1-T (Optimized Kappa Casein Truncated version 1), iii) OBC1 (Optimized Beta Casein version 1), iv) OBC1-T (Optimized Beta Casein Truncated version 1), v) OS1C1 (Optimized alpha S1 Casein version 1), vi) OS1C1-T (Optimized alpha S1 Casein Truncated version 1), vii) OS2C1 (Optimized alpha S2 Casein version 1), and viii) OS2C1-T (Optimized alpha S2 Casein Truncated version 1). In these constructs, each transgene is controlled under a soybean constitutive GmSM8-1 promoter. Two constructs for expressing truncated α-S1 casein and truncated α-S2 casein can be generated according to the disclosure. The protein expression was detected visually by GFP expression under blue light and measured by western blot analysis using Anti-6×His tag antibody.

Third, a soybean tissue-specific AR-Pro3 promoter was tested to drive individual transgene fused with GFP and 6×His-tag and express GFP-fusion milk proteins including α-S1 casein, α-S2 casein, β-casein, κ-casein. Six constructs were generated out of eight expression cassettes illustrated in FIG. 3C. Eight distinct types of transgenes encoding milk proteins. Each transgene are labeled as i) OKC1 (Optimized Kappa Casein version 1), ii) OKC1-T (Optimized Kappa Casein Truncated version 1), iii) OBC1 (Optimized Beta Casein version 1), iv) OBC1-T (Optimized Beta Casein Truncated version 1), v) OS1C1 (Optimized alpha S1 Casein version 1), vi) OS1C1-T (Optimized alpha S1 Casein Truncated version 1), vii) OS2C1 (Optimized alpha S2 Casein version 1), and viii) OS2C1-T (Optimized alpha S2 Casein Truncated version 1). Two constructs for expressing truncated α-S1 casein and truncated α-S2 casein can be generated according to the disclosure. The protein expression was detected visually by GFP expression under blue light and measured by western blot analysis using Anti-His tag antibody.

After transgene expression vectors/plasmids were constructed, the vectors/plasmids were then introduced into A. tumefaciens LBA4404 and A. tumefaciens GV3101 cells and stored at −80C for transformation use. The same protocol was adopted for all other recombinant expression vectors disclosed in FIGS. 2A-2B, and 3A-3C.

Table 2 is a list of codon-optimized DNA sequences, including OKC1 (SEQ ID NO:1), OKC1-T (SEQ ID NO:2), OBC1 (SEQ ID NO:3), OBC1-T (SEQ ID NO:4), OS1C1 (SEQ ID NO:9), OS2C1 (SEQ ID NO:10), OLA1 (SEQ ID NO:19), OLG1 (SEQ ID NO:20), and OLY1 (SEQ ID NO:21).

Table 3 is a list of protein sequences listed as SEQ ID NO:5, SEQ ID NO:6, SEQ ID NO:7, SEQ ID NO:8, SEQ ID NO:11, SEQ ID NO:12, SEQ ID NO:22, SEQ ID NO:23, and SEQ ID NO:24. These protein sequences are machine-translated using an ExPASy Bioinformatics Resource Portal tool from codon-optimized nucleotide sequences, listed as SEQ ID NO:1, SEQ ID NO:2, SEQ ID NO:3, SEQ ID NO:4, SEQ ID NO:9, SEQ ID NO:10, SEQ ID NO:19, SEQ ID NO:20, and SEQ ID NO:21, respectively.

TABLE 2
List of Codon-Optimized DNA Sequences

| Sequence | | Sequence | |
|---|---|---|---|
| Identifier | Nucleotide Sequence | Identifier | Nucleotide Sequence |
| SEQ ID | ATGATGAAATCTTTTTTTCTCGTT | SEQ ID | ATGCAAGAGCAGAATCAAGAGC |
| NO: 1 | GTAACTATTTTGGCTTTGACTTTG | NO: 2 | AGCCAATCCGTTGTGAGAAGGAC |
| OKC1 | CCCTTTCTTGGAGCACAAGAGCA | OKC1-T | GAGAGGTTCTTCTCAGACAAGAT |
| sequence | GAATCAAGAGCAGCCAATCCGTT | sequence | CGCCAAATATATACCCATACAAT |
| (573 bp) | GTGAGAAGGACGAGAGGTTCTTC | (513 bp) | ATGTACTCTCACGCTACCCTAGCT |
| | TCAGACAAGATCGCCAAATATAT | | ACGGGCTTAACTACTATCAGCAA |
| | ACCCATACAATATGTACTCTCAC | | AAACCTGTAGCACTGATAAATAA |
| | GCTACCCTAGCTACGGGCTTAAC | | CCAGTTTCTCCCCTATCCCTATTA |
| | TACTATCAGCAAAAACCTGTAGC | | TGCTAAACCTGCCGCCGTGAGGA |
| | ACTGATAAATAACCAGTTTCTCC | | GTCCAGCACAAATACTTCAGTGG |
| | CCTATCCCTATTATGCTAAACCT | | CAAGTGCTCAGTAACACCGTGCC |
| | GCCGCCGTGAGGAGTCCAGCAC | | AGCAAAAAGCTGCCAGGCTCAGC |
| | AAATACTTCAGTGGCAAGTGCTC | | CCACCACAATGGCCCGTCATCCC |
| | AGTAACACCGTGCCAGCAAAAA | | CATCCTCACCTTAGCTTCATGGCA |
| | GCTGCCAGGCTCAGCCCACCACA | | ATCCCACCAAAGAAGAATCAAGA |
| | ATGGCCCGTCATCCCCATCCTCA | | CAAGACCGAAATACCTACCATCA |
| | CCTTAGCTTCATGGCAATCCCAC | | ACACAATTGCATCTGGAGAGCCT |
| | CAAAGAAGAATCAAGACAAGAC | | ACCAGTACACCAACAACTGAGGC |
| | CGAAATACCTACCATCAACACAA | | AGTAGAGTCTACTGTTGCTACCCT |
| | TTGCATCTGGAGAGCCTACCAGT | | TGAGGACAGCCCCGAGGTTATAG |
| | ACACCAACAACTGAGGCAGTAG | | AGTCCCCACCTGAGATAAATACC |
| | AGTCTACTGTTGCTACCCTTGAG | | GTGCAGGTGACAAGTACCGCCGT |
| | GACAGCCCCGAGGTTATAGAGTC | | ATAA |
| | CCCACCTGAGATAAATACCGTGC | | |
| | AGGTGACAAGTACCGCCGTATAA | | |
| SEQ ID | ATGAAGGTCTTGATATTGGCATG | SEQ ID | ATGAGAGAGCTTGAGGAACTCAA |
| NO: 3 | TCTGGTAGCCTTGGCACTGGCTA | NO: 4 | CGTACCAGGGGAGATTGTAGAAT |
| OBC1 | GAGAGCTTGAGGAACTCAACGT | OBC1-T | CTCTTTCCAGCAGCGAGGAGAGT |
| sequence | ACCAGGGGAGATTGTAGAATCTC | sequence | ATCACAAGGATTAATAAGAAAAT |
| (675 bp) | TTTCCAGCAGCGAGGAGAGTATC | (633 bp) | CGAAAAGTTCCAAAGTGAGGAAC |
| | ACAAGGATTAATAAGAAAATCG | | AACAACAAACCGAAGACGAACTT |
| | AAAAGTTCCAAAGTGAGGAACA | | CAAGACAAAATACACCCATTTGC |
| | ACAACAAACCGAAGACGAACTT | | ACAGACACAATCACTCGTGTATC |
| | CAAGACAAAATACACCCATTTGC | | CATTCCCAGGACCAATCCCAAAC |
| | ACAGACACAATCACTCGTGTATC | | AGTTTGCCTCAAAACATACCTCC |
| | CATTCCCAGGACCAATCCCAAAC | | ACTGACTCAAACTCCAGTTGTGG |
| | AGTTTGCCTCAAAACATACCTCC | | TACCTCCTTTCCTGCAACCAGAA |
| | ACTGACTCAAACTCCAGTTGTGG | | GTGATGGGTGTCTCAAAAGTAAA |
| | TACCTCCTTTCCTGCAACCAGAA | | GGGGGCAATGGCTCCTAAACATA |
| | GTGATGGGTGTCTCAAAAGTAAA | | AAGAGATGCCTTTCCCTAAGTAT |
| | GGGGGCAATGGCTCCTAAACATA | | CCAGTGGAACCACTGACTGAGAG |
| | AAGAGATGCCTTTCCCTAAGTAT | | TCAATCACTTACCCTCACTGATGT |
| | CCAGTGGAACCACTGACTGAGA | | AGAGAACCTTCACCTCCCCCTGC |
| | GTCAATCACTTACCCTCACTGAT | | CTTTGCTGCAAAGCTGGATGCAC |
| | GTAGAGAACCTTCACCTCCCCCT | | CAACCTCATCAGCCTCTTCCTCCC |
| | GCCTTTGCTGCAAAGCTGGATGC | | ACCGTTATGTTTCCCCCTCAGTCC |
| | ACCAACCTCATCAGCCTCTTCCT | | GTCCTGAGTTTGAGTCAGTCCAA |
| | CCCACCGTTATGTTTCCCCCTCA | | AGTCTTGCCTGTTCCCCAAAAAG |
| | GTCCGTCCTGAGTTTGAGTCAGT | | CAGTGCCATACCCACAAAGGGAC |
| | CCAAAGTCTTGCCTGTTCCCCAA | | ATGCCAATACAAGCATTCCTCCTT |
| | AAAGCAGTGCCATACCCACAAA | | TACCAGGAGCCCGTACTCGGCCC |
| | GGGACATGCCAATACAAGCATTC | | TGTGCGTGGTCCTTTCCCTATTAT |
| | CTCCTTTACCAGGAGCCCGTACT | | AGTCTAA |
| | CGGCCCTGTGCGTGGTCCTTTCC | | |
| | CTATTATAGTCTAA | | |
| SEQ ID | ATGAAACTTCTCATTCTTACCTGT | SEQ ID | ATGAAGTTTTTTATCTTTACCTGT |
| NO: 9 | CTGGTGGCTGTAGCTCTCGCCCG | NO: 10 | TTGCTTGCAGTCGCCTTGGCCAA |
| OS1C1 | CCCAAAACATCCCATAAAACATC | OS2C1 | GAATACTATGGAACACGTAAGCT |
| sequence | AAGGATTGCCCCAGGAAGTACTC | sequence | CAAGTGAAGAATCTATAATAAGT |
| (645 bp) | AACGAGAATCTCCTCCGTTTTTT | (669 bp) | CAAGAGACATATAAGCAAGAGA |
| | CGTTGCTCCTTTCCCCGAAGTGTT | | AAAACATGGCAATAAATCCCTCC |
| | CGGGAAGGAAAAAGTAAACGAG | | AAGGAGAATCTTTGTAGCACTTT |
| | CTTTCAAAGGACATCGGCTCTGA | | TTGCAAAGAAGTTGTGAGAAATG |
| | AAGTACCGAGGATCAGGCTATG | | CAAATGAGGAAGAATACTCAATA |
| | GAAGATATCAAGCAAATGGAGG | | GGCAGCTCTTCCGAAGAATCTGC |
| | CCGAATCTATAAGTTCTTCAGAA | | TGAAGTCGCTACTGAAGAGGTCA |
| | GAAATAGTTCCCAACTCAGTGGA | | AAATAACAGTTGACGACAAGCAT |
| | GCAGAAGCACATTCAGAAAGAA | | TATCAAAAAGCCCTGAATGAAAT |
| | GACGTGCCCAGCGAGCGCTATCT | | AAACCAGTTCTACCAAAAATTTC |
| | GGGATATTTGGAACAGCTGCTCA | | CCCAATACCTCCAGTACCTTTATC |
| | GACTGAAAAAGTACAAGGTGCC | | AAGGACCCATAGTCCTCAACCCT |
| | TCAGCTCGAAATCGTACCCAATA | | TGGGATCAGGTCAAGCGTAATGC |
| | GTGCTGAAGAAAGGTTGCACTCA | | TGTTCCAATAACACCAACACTCA |
| | ATGAAAGAGGGGATTCACGCAC | | ATCGTGAACAACTGTCTACCTCA |
| | AACAAAAAGAGCCTATGATCGG | | GAAGAAAATTCCAAAAAAACTGT |
| | AGTAAATCAAGAACTGGCATACT | | GGATATGGAAAGTACAGAAGTTT |
| | TTTATCCCGAGTTGTTTCGCCAAT | | TTACTAAAAAGACCAAGCTCACC |
| | TCTATCAACTGGATGCCTACCCT | | GAGGAGGAAAAAAATAGATTGA |
| | TCCGGTGCATGGTACTACGTACC | | ATTTTCTTAAGAAGATCAGTCAA |
| | CCTCGGTACTCAATATACCGATG | | CGCTATCAGAAGTTCGCCCTTCC |
| | CTCCCTCCTTTTCCGACATTCCTA | | ACAATACCTCAAGACTGTATACC |
| | ATCCTATAGGTTCCGAGAATAGC | | AACATCAGAAGGCCATGAAGCCT |
| | GAAAAGACCACCATGCCCTTATG | | TGGATTCAGCCCAAAACAAAGGT |
| | GTAA | | AATCCCCTATGTTAGATACTTGTA |
| | | | A |
| SEQ ID | ATGATGAGTTTCGTTTCTCTCCTG | SEQ ID | ATGAAATGTCTCCTTTTGGCATTG |
| NO: 19 | CTCGTGGGTATTCTTTTTCATGCA | NO: 20 | GCACTCACATGCGGAGCACAAGC |
| OLA1 | ACACAGGCTGAACAACTGACCA | OLG1 | CTTGATCGTAACACAGACTATGA |
| sequence | AGTGCGAAGTATTCAGGGAACTG | sequence | AGGGTCTTGATATACAGAAGGTG |
| (429 bp) | AAGGACCTTAAAGGGTATGGCG | (537 bp) | GCCGGGACTTGGTACAGTTTGGC |
| | GCGTGTCTCTGCCTGAGTGGGTA | | AATGGCCGCATCCGACATCTCCT |
| | TGTACTACTTTTCATACATCAGG | | TGTTGGACGCACAATCAGCCCCA |
| | GTATGACACCCAAGCTATTGTTC | | TTGCGTGTGTACGTAGAAGAGCT |
| | AGAACAATGATTCAACTGAATAT | | TAAACCAACTCCCGAGGGGGATC |
| | GGTTTGTTCCAGATAAATAATAA | | TGGAAATTCTGCTCCAGAAATGG |
| | AATTTGGTGCAAAGACGACCAA | | GAGAACGGTGAGTGCGCCCAGAA |
| | AACCCTCACAGCAGCAACATTTG | | GAAGATCATCGCAGAGAAGACCA |
| | CAACATCTCCTGTGATAAATTTC | | AAATTCCAGCAGTATTCAAAATC |
| | TGGACGATGACCTGACCGACGAC | | GACGCATTGAACGAAAATAAGGT |
| | ATCATGTGTGTTAAAAAGATTCT | | GCTCGTACTGGACACTGATTATA |
| | CGATAAGGTCGGTATTAACTATT | | AGAAGTATCTCCTTTTCTGTATGG |
| | GGCTCGCTCACAAGGCATTGTGC | | AGAACTCAGCAGAGCCTGAACAG |
| | AGTGAGAAACTTGATCAATGGCT | | AGTCTTGCCTGCCAATGCCTTGTT |
| | CTGTGAAAAACTTTGA | | CGTACCCCAGAGGTAGATGATGA |
| | | | AGCTCTGGAAAAGTTCGATAAGG |
| | | | CCCTTAAGGCTCTGCCTATGCAC |
| | | | ATTAGGCTTTCTTTCAATCCAACT |
| | | | CAACTTGAGGAACAATGTCACAT |
| | | | TTAG |
| SEQ ID | ATGAAAGCCCTCCTGATCGTGGG | | |
| NO: 21 | TCTGCTCCTCTTGAGCGTTGCAG | | |
| OLY1 | TACAGGGTAAGAAATTTCAGCGT | | |
| sequence | TGCGAACTTGCCAGGACACTGAA | | |
| (447 bp) | GAAACTTGGGCTCGACGGGTATC | | |
| | GTGGAGTGTCATTGGCTAACTGG | | |
| | GTCTGTCTTGCACGTTGGGAATC | | |
| | CAATTACAACACTCGTGCCACCA | | |
| | ACTACAACCGTGGCGATAAGTCC | | |
| | ACCGATTATGGCATCTTTCAGAT | | |
| | TAACTCTCGCTGGTGGTGTAACG | | |
| | ATGGCAAAACCCCCAAGGCAGT | | |
| | AAACGCCTGTAGGATCCCATGTT | | |
| | CTGCTTTGCTCAAGGACGATATA | | |
| | ACACAGGCTGTGGCTTGCGCCAA | | |
| | GAGAGTAGTACGTGATCCCCAAG | | |
| | GTATCAAGGCTTGGGTCGCTTGG | | |
| | AGGAACAAGTGTCAGAATAGAG | | |
| | ACTTGCGCAGCTATGTACAAGGC | | |
| | TGTCGCGTTTAA | | |

TABLE 3
List of Protein Sequences

| Sequence | Protein Sequence | Sequence | Protein Sequence |
|---|---|---|---|
| Identifier | (Amino Acid) | Identifier | (Amino Acid) |
| SEQ ID | MMKSFFLVVTILALTLPFLGA | SEQ ID | MQEQNQEQPIRCEKDERFFSDKI |
| NO: 5 | QEQNQEQPIRCEKDERFFSDK | NO: 6 | AKYIPIQYVLSRYPSYGLNYYQQ |
| | IAKYIPIQYVLSRYPSYGLNY | | KPVALINNQFLPYPYYAKPAAVR |
| | YQQKPVALINNQFLPYPYYA | | SPAQILQWQVLSNTVPAKSCQAQ |
| | KPAAVRSPAQILQWQVLSNT | | PTTMARHPHPHLSFMAIPPKKNQ |
| | VPAKSCQAQPTTMARHPHPH | | DKTEIPTINTIASGEPTSTPTTEAV |
| | LSFMAIPPKKNQDKTEIPTINT | | ESTVATLEDSPEVIESPPEINTVQV |
| | IASGEPTSTPTTEAVESTVATL | | TSTAV* |
| | EDSPEVIESPPEINTVQVTSTA | | |
| | V* | | |
| SEQ ID | MKVLILACLVALALARELEEL | SEQ ID | MRELEELNVPGEIVESLSSSEESIT |
| NO: 7 | NVPGEIVESLSSSEESITRINKK | NO: 8 | RINKKIEKFQSEEQQQTEDELQD |
| | IEKFQSEEQQQTEDELQDKIH | | KIHPFAQTQSLVYPFPGPIPNSLPQ |
| | PFAQTQSLVYPFPGPIPNSLPQ | | NIPPLTQTPVVVPPFLQPEVMGVS |
| | NIPPLTQTPVVVPPFLQPEVM | | KVKGAMAPKHKEMPFPKYPVEP |
| | GVSKVKGAMAPKHKEMPFP | | LTESQSLTLTDVENLHLPLPLLQS |
| | KYPVEPLTESQSLTLTDVENL | | WMHQPHQPLPPTVMFPPQSVLSL |
| | HLPLPLLQSWMHQPHQPLPPT | | SQSKVLPVPQKAVPYPQRDMPIQ |
| | VMFPPQSVLSLSQSKVLPVPQ | | AFLLYQEPVLGPVRGPFPIIV* |
| | KAVPYPQRDMPIQAFLLYQEP | | |
| | VLGPVRGPFPIIV* | | |
| SEQ ID | MKLLILTCLVAVALARPKHPI | SEQ ID | MKFFIFTCLLAVALAKNTMEHVS |
| NO: 11 | KHQGLPQEVLNENLLRFFVA | NO: 12 | SSEESIISQETYKQEKNMAINPSK |
| | PFPEVFGKEKVNELSKDIGSE | | ENLCSTFCKEVVRNANEEEYSIGS |
| | STEDQAMEDIKQMEAESISSS | | SSEESAEVATEEVKITVDDKHYQ |
| | EEIVPNSVEQKHIQKEDVPSE | | KALNEINQFYQKFPQYLQYLYQG |
| | RYLGYLEQLLRLKKYKVPQL | | PIVLNPWDQVKRNAVPITPTLNR |
| | EIVPNSAEERLHSMKEGIHAQ | | EQLSTSEENSKKTVDMESTEVFT |
| | QKEPMIGVNQELAYFYPELFR | | KKTKLTEEEKNRLNFLKKISQRY |
| | QFYQLDAYPSGAWYYVPLGT | | QKFALPQYLKTVYQHQKAMKP |
| | QYTDAPSFSDIPNPIGSENSEK | | WIQPKTKVIPYVRYL* |
| | TTMPLW* | | |
| SEQ ID | MMSFVSLLLVGILFHATQAE | SEQ ID | MKCLLLALALTCGAQALIVTQT |
| NO: 22 | QLTKCEVFRELKDLKGYGGV | NO: 23 | MKGLDIQKVAGTWYSLAMAAS |
| | SLPEWVCTTFHTSGYDTQAIV | | DISLLDAQSAPLRVYVEELKPTPE |
| | QNNDSTEYGLFQINNKIWCK | | GDLEILLQKWENGECAQKKIIAE |
| | DDQNPHSSNICNISCDKFLDD | | KTKIPAVFKIDALNENKVLVLDT |
| | DLTDDIMCVKKILDKVGINY | | DYKKYLLFCMENSAEPEQSLACQ |
| | WLAHKALCSEKLDQWLCEK | | CLVRTPEVDDEALEKFDKALKAL |
| | L* | | PMHIRLSFNPTQLEEQCHI* |
| SEQ ID | MKALLIVGLLLLSVAVQGKK | | *: Translation stop |
| NO: 24 | FQRCELARTLKKLGLDGYRG | | |
| | VSLANWVCLARWESNYNTR | | |
| | ATNYNRGDKSTDYGIFQINSR | | |
| | WWCNDGKTPKAVNACRIPCS | | |
| | ALLKDDITQAVACAKRVVRD | | |
| | PQGIKAWVAWRNKCQNRDL | | |
| | RSYVQGCRV* | | |

### Example 2. Transient Expression of Milk Proteins (Casein and Whey) in Tobacco and Soybean

Sequence information of four types of casein protein (α-S1, α-S2, β, κ) were collected from NCBI (GenBank accession No. ACG63494.1 (SEQ ID NO:15), NP_776953.1 (SEQ ID NO:16), AGT56763.1 (SEQ ID NO:17), AAQ87923.1 (SEQ ID NO:18)), as well as three types of whey protein (α-lactalbumin, β-lactoglobulin, lysozyme) were collected from NCBI (GenBank accession No NP_776803.1 (SEQ ID NO:25), NP_776354.2 (SEQ ID NO:26), NP_001071297.1 (SEQ ID NO:27)), representing sequence information of seven bovine milk proteins. Then, the sequences obtained from GenBank were optimized based on soybean codon usage frequency (idtdna.com/CodonOpt). See, for examples, Plotkin et al., Nat. Rev. Genet. 12:32-42, 2011; Sharp, Nucl. Acids. Res. 15:1281-1295, 2009; and Cannarozzi et al., Cell 141:355-367, 2010. Each synthesized fragment from the soybean codon-optimized DNA sequences encoding milk proteins was ligated into pCambia1305.1 binary vector which has either GUSPlus or GFP, followed by 6×His tag at 3′ end separately using Infusion ligation (In-Fusion® HD Cloning System CE, www.clontech.com).

The optimized transgene fragments inserted into the vector are provided in FIGS. 2A-3C and Table 2. In case of the truncated versions of OKC1-T, OBC1-T, OS1C1-T, and 052C1-T constructs, signal sequences are removed from the original versions, which are OKC1, OBC1, OS1C1, and 052C1, respectively, for further functional analyses.

For the transient transformation, Agrobacterium strain was prepared. Each transgene construct in the pCambia1305.1 vector was introduced into Agrobacterium strain LBA4404 or GV3101 separately. A couple of single colonies containing the provided transgene construct of each were inoculated into 5 ml LB medium with 50 mg/l Kanamycin and 100 mg/l streptomycin and were incubated overnight at 30 degree with shaking at 150 rpm. The growing cell cultures were then used to inoculate 180 ml of LB media (50 mg/l Kanamycin and 100 mg/l streptomycin) for subculture under the same growing condition for 20 hours. The Agrobacterium cells were collected by centrifugation at 3000 g for 10 minutes. Cell pellets were resuspended in 90 ml MS media and centrifuged again. These resuspension and centrifugation steps were repeated to wash Agrobacterium cells. Final cell pellets were resuspended in 90 ml infiltration buffer (MS media with 0.1 mM acetosyringone and 0.5 mM DTT).

For the tobacco plants, the syringe infiltration method was used. The agrobacterium resuspension culture was pressure-infiltrated into the abaxial or adaxial surface of N. tabacum (cultivar Xanthi) leaves using a 1 ml sterile syringe for the tobacco plants. 4-6 weeks old tobacco N. tabacum (cultivar Xanthi) leaves were used for syringe infiltration.

For the soybean plants, each soybean seedling was put in a plastic bag containing 45 ml bacteria suspension as described above, and the whole bag was put in a sonicator (3 L digital ultrasonic cleaner) and sonicated at 40 kHz for 30 s while the leaves were merged in the buffer. The seedlings were taken out of the sonicator and submerged in a glass flask contained 45 ml bacteria suspension. The flask was set placed in a 2 gallons vacuum chamber attached to a 3CFM single stage vacuum pump and exposed to three 3-minute periods of vacuum to facilitate the plant uptake of bacteria suspension. After the infiltration, seedlings were transferred to soil and grown for three days.

The experiments transiently expressing milk proteins including casein and whey proteins were performed with soybean cultivars including Williams 82 and Wyandot 14. Specifically, both soybean cultivars, Williams 82 and Wyandot 14, were germinated on pre-moistened filter paper, and later transferred to soil. For normal growth, 18-hour day and 6-hour night photoperiods were provided without interruption during the entire germination and seedling growth process.

### Example 3. Visual Detection of Transient Expression of Milk Proteins (Casein and Whey) in Tobacco and Soybean

Since GUSPlus™ was fused with codon-optimized transgenes of interest in each construct, histochemical staining of beta-Glucuronidase (GUS) was used to evaluate transient expression in the tobacco leaves, and also the soybean seedlings. 3 days after the agro-infiltration, leaves were taken off from the seedlings and submerged in the staining buffer provided by beta-Glucuronidase (GUS) reporter gene staining kit (sigma, www.sigmaaldrich.com/united-states.html). After 24 hours of staining and 6 hours distaining, the leaves were scanned and blue areas would be calculated by publicly-available ImageJ program.

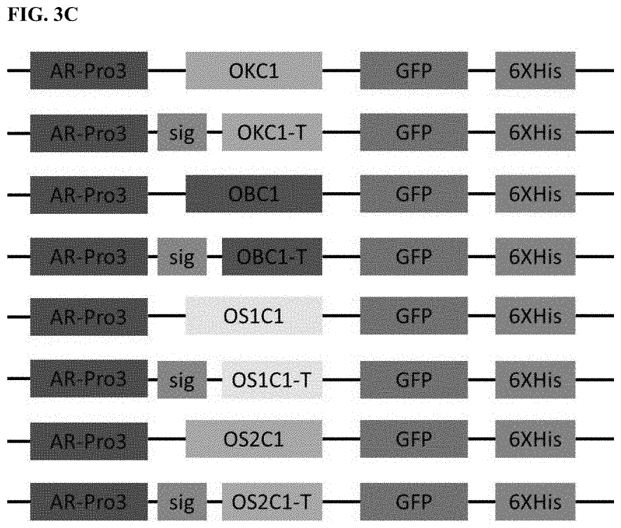

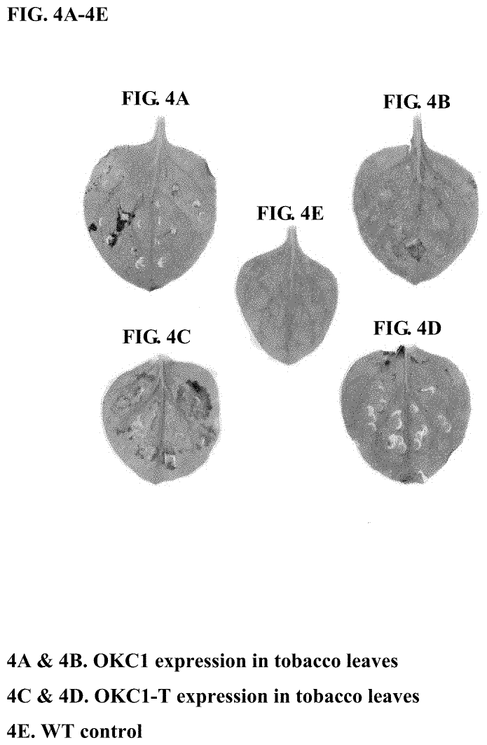

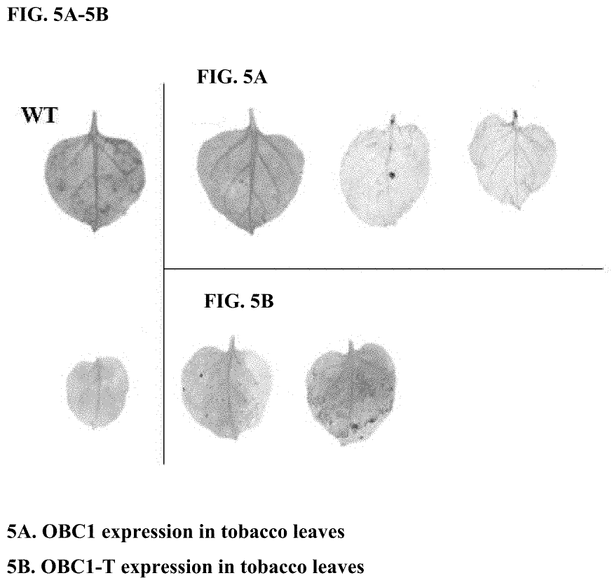

Infiltration experiments using Agrobacterium carrying recombinant expression vectors illustrated in FIGS. 2A and 2B showed successful transient expressions of milk proteins including κ-casein protein (OKC1), truncated κ-casein protein without signal peptide (OKC1-T), β-casein protein (OBC1), truncated β-casein protein without signal peptide (OBC1-T), α-lactalbumin (OLA1), β-lactoglobulin (OLG1) in independent experimental setting. FIG. 4A-4E illustrates obvious GUS staining in the tobacco leaves, signifying successful expression of κ-casein protein (FIGS. 4A and 4B) and κ-casein protein without signal peptide (FIGS. 4C and 4D), compared to a WT control (FIG. 4E). FIG. 5A-5B shows apparent GUS staining, indicating expressions of both β-casein protein (FIG. 5A) and β-casein protein without signal peptide (FIG. 5B) in the tobacco leaves. FIG. 6A-6B shows expressions of whey proteins, α-lactalbumin (FIG. 6A) and β-lactoglobulin (FIG. 6B), compared to a WT control. The GUS staining is displayed such as dots, spots, stains with distinctively dark color in FIGS. 4A-6B.

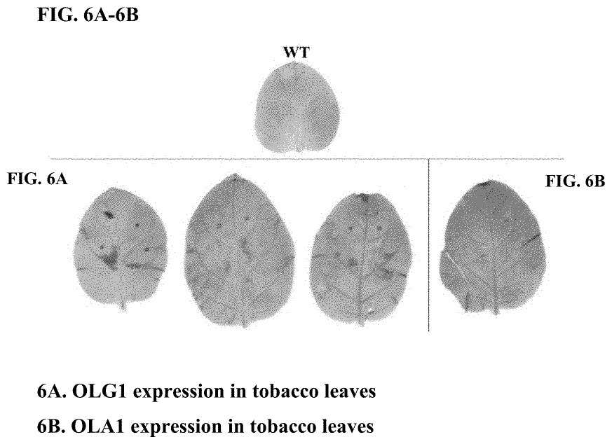

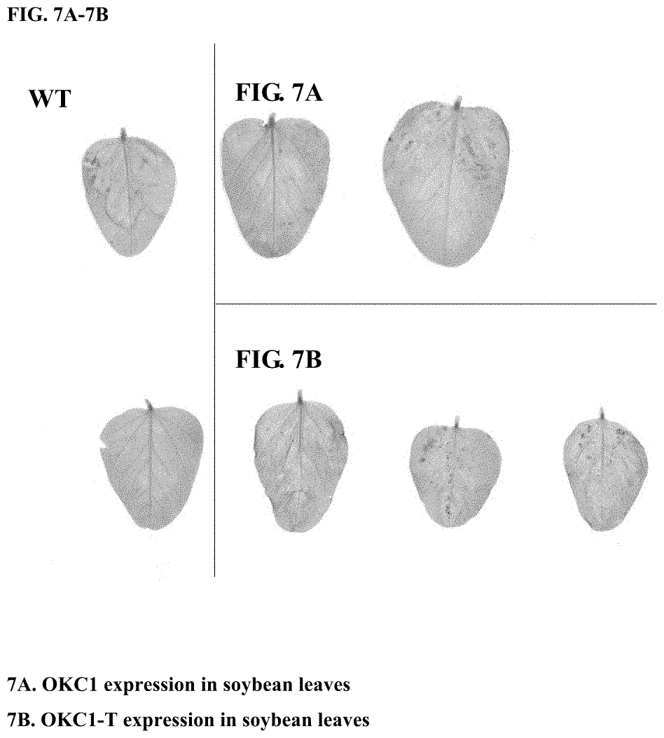

In soybean leaves, transient expressions of milk proteins including κ-casein protein (OKC1), κ-casein protein without signal peptide (OKC1-T), β-casein protein (OBC1), and β-casein protein without signal peptide (OBC1-T), were detected in independent experimental setting. FIG. 7A-7B illustrates obvious GUS staining in the soybean leaves, indicating expression of κ-casein protein (FIG. 7A) and κ-casein protein without signal peptide (FIG. 7B), compared to a WT control. FIG. 8A-8B also illustrates apparent GUS staining, showing expressions of both β-casein protein (FIG. 8A) and β-casein protein without signal peptide (FIG. 8B). The GUS staining is displayed such as dots, spots, stains with distinctively dark color in FIGS. 7A-8B.

### Example 4. Visual Detection of Stable Expression of Milk Proteins in Tobacco (N. tabacum) and Rice (O. sativa)

For stable transformation, Agrobacterium strain was prepared. Each transgene construct in the pCambia1305.1 vector was introduced into Agrobacterium strain LBA4404 separately and glycerol freezer stock was prepared from a single bacterial colony containing the provided transgene construct. 40-50 ul of each glycerol stock were inoculated into 20 ml of MGL medium (pH 7.0) with Kanamycin and streptomycin separately and were incubated overnight at 28 degree with shaking at 250 rpms. 5 ml of the growing cell cultures were then used to inoculate 15 ml of TY medium containing the same antibiotics plus acetosyringone for subculture under the same growing condition overnight. Dilute the overnight culture by adding 1.5 ml of the culture to 20 ml TY medium (pH 5.5) containing 200 uM acetosyringone. O.D at 600 nm should be within the range of 0.1 to 0.2.

For stable tobacco transgenic lines, the tobacco NT1 leaves were cut into 1 cm² squares and suspended in the Agrobacterium solution soaking for 10 mins. Place leaf pieces abaxial side down in petridish containing co-cultivation MS medium modified with 30 g/L sucrose, 2.0 mg/L kinetin, 2.0 mg/L IAA and 200 uM acetosyringone pH 5.6-5.8. After 3 days of co-cultivation period, transfer leaf pieces to induction medium consisting of MS medium modified with 30 g/L sucrose, 2.0 mg/L kinetin, 2.0 mg/L IAA, 400 mg/L carbenicillin, 250 mg/L cefotaxime, and 250 mg/L kanamycin sulfate. The plates were incubated for 10 days and then subculture to fresh medium of the same formulation. Subculture tissue every 21 days until shoots form. When transferred to rooting medium modified with 0.2 mg/L IBA, shoots should root in about 14 days. The stable tobacco transformation technique is well known to a person of ordinary skill in the art. The transformation protocol can be obtained from UC Davis plant transformation facility (ucdptf.ucdavis.edu/services).

For stable rice transgenic lines, Agrobacterium-mediated transformation was conducted in mature seed-derived callus tissues of japonica and/or indica rice cultivars. The rice transformation was performed by the following methods described in Hiei, Y., & Komari, T. (2008) “Agrobacterium-mediated transformation of rice using immature embryos or calli induced from mature seed” Nature Protocols, 3(5), 824.

To select plants expressing transgene constructs provided in this disclosure, antibiotic selection media was used for further growth of regenerated shoots, and the regenerated plants expressing the constructs demonstrated in this disclosure successfully grew.

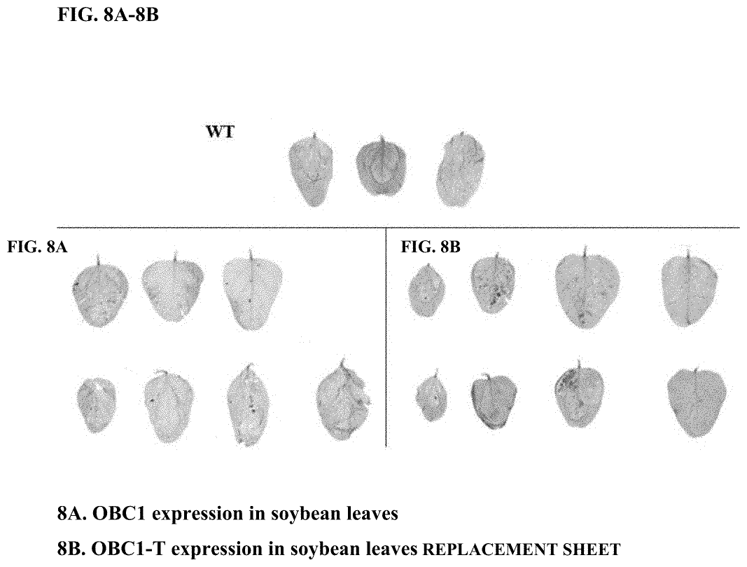

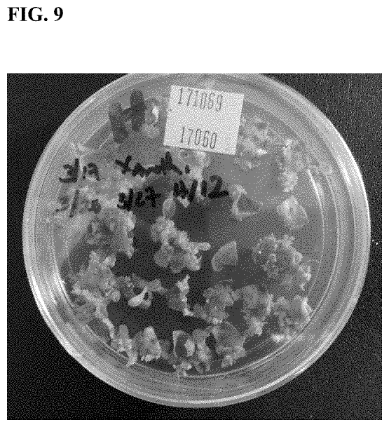

The protocol used for GUS staining described in Example 3 was used to visualize GUS activity in the results disclosed in Example 4. FIG. 9 shows successfully-regenerated tobacco plants, which suggests stable expression of κ-casein protein. From the selection media, seven plants fully regenerated with expression of 35S:OKC1:GUS construct, and ten stably-transformed plants expressed 35S:OKC1-T:GUS. FIG. 10A-10B confirms stable expression of κ-casein protein by the expression of GUS protein that is fused to κ-casein protein. The expression of GUS protein indicates the expression of the fusion protein between κ-casein and GUS. FIGS. 10A and 10B show OKC1 and OKC1-T expression in stable transgenic tobacco leaves, respectively. The GUS staining is displayed such as dots, spots, stains with distinctively dark color in FIG. 10A-10B.

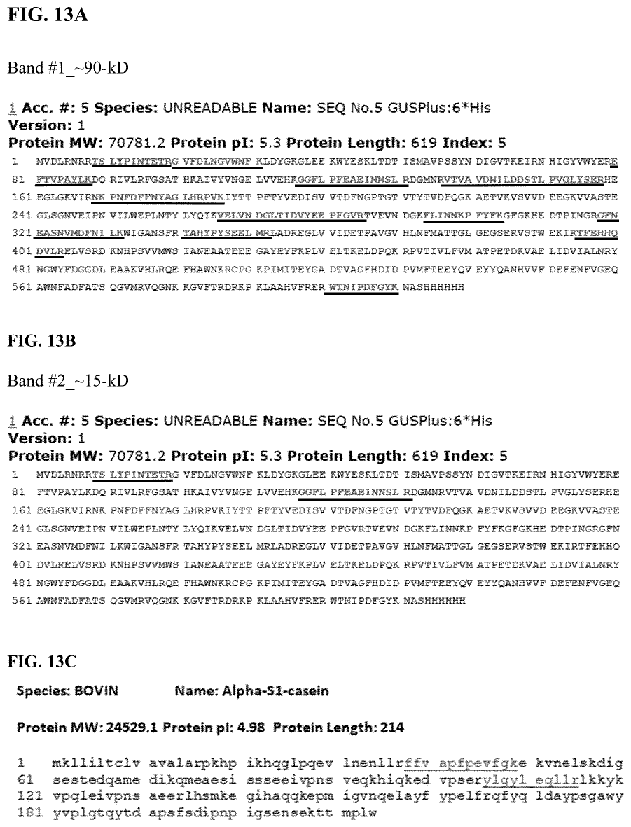

FIG. 14A shows stable expression of truncated κ-casein protein by the expression of GUS protein in stable transgenic rice leaves. The GUS staining is displayed such as dots, spots, stains with distinctively dark color in FIG. 14A.

Stable T₀ transgenic tobacco plants expressing two (truncated and full-length) versions of κ-casein protein, and stable T₀ transgenic rice plants expressing the truncated κ-casein were generated through Agrobacterium-mediated transformation as shown in FIGS. 10A-10B and 14. The leaves of the primary transformants (T₀) were GUS-positive. The seed from the self-fertilized T₀ plants were viable. The resulting T₁ seedlings harboring the transgene can be tested to confirm GUS-positive staining.

### Example 5. Molecular Detection of Stable Expression of Milk Proteins in Tobacco and Rice

To analyze protein expression molecularly and semi-quantitatively, western blot analysis was performed on protein extracts from plants described herein. All the plant species tested including tobacco, soybean, rice, and embryogenic callus of lima bean were transiently and/or stably transformed with recombinant expression vectors disclosed in FIGS. 1, 2A-2B and 3A-3C. The western blot analysis was carried out by the protocol generally well-known to persons with the ordinary skill in the art.

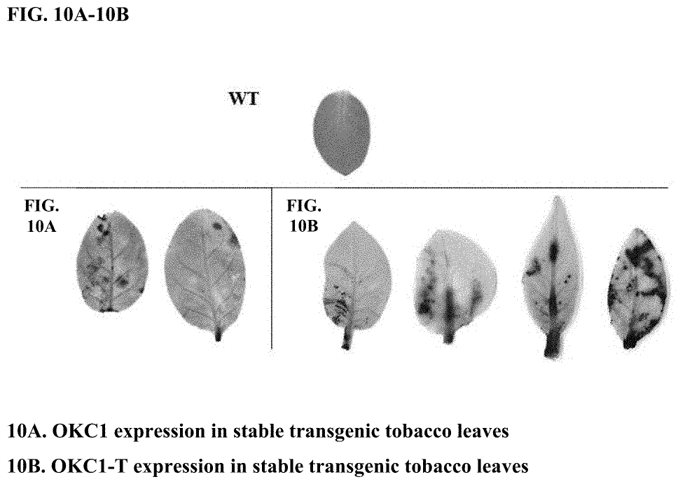

Protein lysates were extracted from stable transgenic tobacco leaf tissues (FIG. 10B) in 50 ul extraction buffer (100 mM EDTA pH 8.0, 120 mM Tris-HCl pH 6.8, 4% SDS, 12% Sucrose, 200 mM DTT) per 10 mg tissue. Lysate (about 50 ug of protein per well) was used for western blot analysis. As a reference, a protein extract was prepared of wilt-type tobacco plants. The poly-epitope control used in western blots, includes GFP, 6×His, FLAG and Myc epitopes. The poly-epitope control has an expected size of 90 kDa and the gel well was loaded with 420 ng of control protein in each blot. FIG. 11 shows that GUS-fused recombinant κ-casein protein without signal peptide were detected in stable transgenic tobacco leaf tissues at ˜90 kDa, while no signal was detected in control plants and the purified bovine κ-casein protein without His-tag. Since a primary antibody against the poly-histidine epitope was used, recombinant milk proteins tagged with 6×His were observed when the recombinant fusion proteins were successfully expressed in the stable tobacco plants. About ˜90 kDa recombinant κ-casein protein without signal peptide was found in stable transgenic tobacco plants, OKC1-T:GUSplus 010 and OKC1-T:GUSplus 011. Expression of recombinant κ-casein proteins in tobacco were further confirmed by SDS-PAGE gel analysis coupled with mass spectrometry (MS) using transgenic leaf tissues.

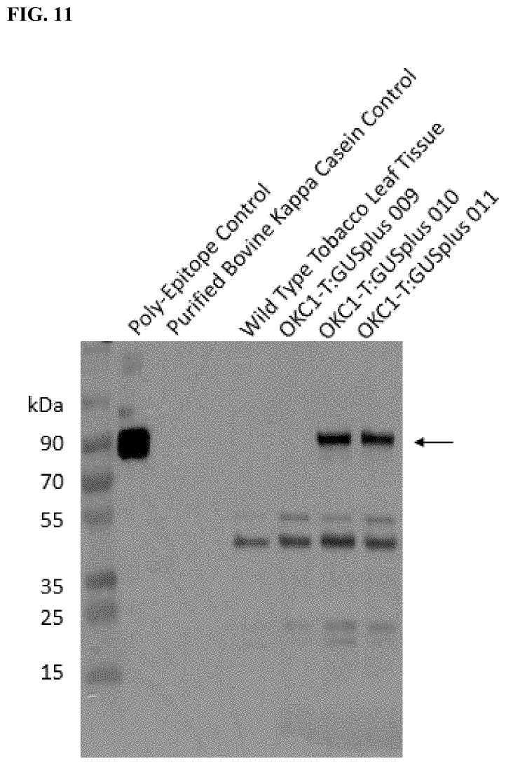

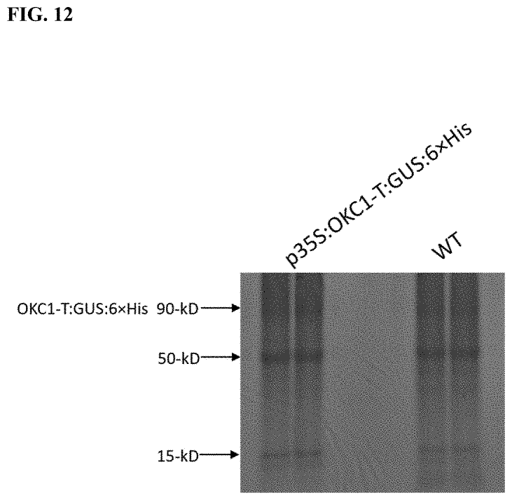

To detect and identify target recombinant proteins in complex biological samples, mass spectrometry was utilized. Proteins extracted from tobacco leaf tissue samples were further concentrated by using 1 g of leaf tissue and enriching for poly-histidine tagged proteins from the lysate using a Ni-NTA affinity column (ThermoFisher Scientific, HisPur Ni-NTA Spin Column). SDS-PAGE analysis was used to separate and resolve proteins isolated from tobacco transgenic leaf tissues. The Optimized Kappa Casein Truncated version 1 (OKC1-T: GUS: 6×His) protein has expected size of ˜90 kDa, along with two other bands at ˜50 kDa and ˜15 kDa, which are illustrated in FIG. 12. All three sizes of bands from transgenic plant sample were excised from the gel, and further processed for mass spectrometry (MS) analysis. As a reference, proteins recovered from WT control tissue were also used for MS analysis. The concentrated proteins were digested with trypsin to produce peptide fragments. Then, the peptide fragments were analyzed via mass spectrometry. Detection of the at least one signature peptide is indicative of presence of target protein in the sample. FIGS. 13A and 13B shows correlation of the determined peptide sequence with the OKC1-T:GUS:6×His protein sequence. Among the 46 peptides specific to the transgenic sample identified from the ˜90 kDa bands, 12 of them matched regions of the OKC1-T:GUS:6×His protein sequence, especially GUS protein sequence that cannot be present in wild-type sample (FIG. 13A). Two out of 31 peptides specific to the transgenic sample from the ˜15 kDa band also matched a portion of the OKC1-T:GUS:6×His protein sequence (FIG. 13B), which could be the result of protein cleavage during treatment. No peptides in the ˜50 kDa band matched to the transgenic peptide sequence.

Similar results were obtained when protein was extracted from lima bean tissue transiently expressing αS1-casein. Two out of five transgenic sample-specific peptides matched recombinant protein sequence from OS1C1:GFP:6×His (OS1C1 comes from Optimized Alpha 51 Casein version 1). Data not provided herein. Peptides identified from mass spec analysis matching αS1-casein were listed in FIG. 13C.

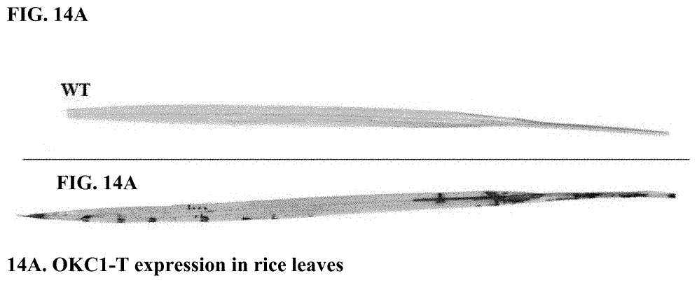

FIG. 15 shows anti-His western blot data detecting expression of truncated recombinant κ-casein protein under the control of the CaMV 35S promoter from stable transgenic rice plants. The experimental procedure for the western blot analysis was identical as described above. Protein lysates were extracted from 80 mg of stable transgenic rice plant leaf tissue, OKC1-T:GUSplus 002, OKC1-T:GUSplus 003, OKC1-T:GUSplus 004. Purified Bovine Kappa Casein was used as a negative control. It was observed that truncated κ-casein protein was expressed in transgenic rice plants, at least OKC-1:GUSplus 003 as shown in FIG. 15.

As illustrated in FIG. 3B, the expression cassettes comprising 1) GmSM8-1 promoter, 2) the codon-optimized milk proteins coding sequences, including α-S1 casein, α-S2 casein, β-casein, κ-casein, 3) GFP as a marker, 4) 6×His were transformed and integrated into the tobacco plants.

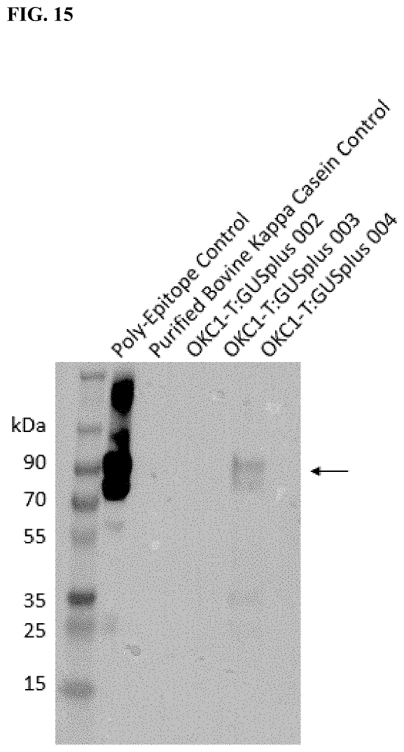

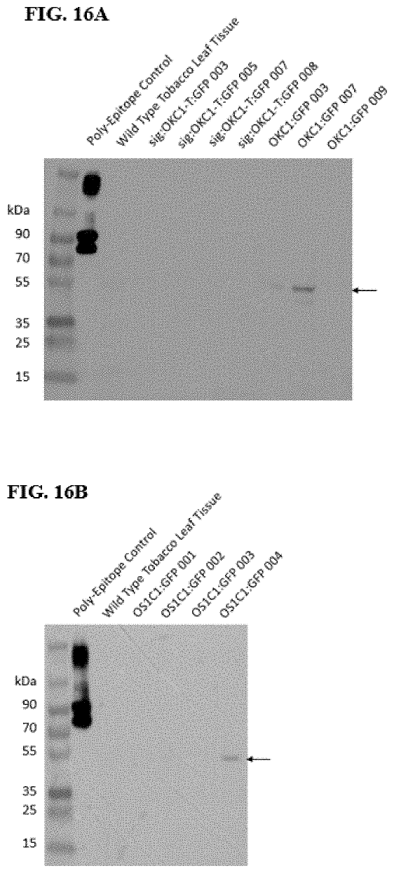

To test stable expression of recombinant milk proteins in tobacco plants, the western blot analyses were performed. FIGS. 16A and 16B show expression of two (truncated and full-length) versions of κ-casein and α-S1 casein proteins under the control of the constitutive GmSM8-1 promoter in stable transgenic tobacco leaf tissues, respectively. Protein lysates for the western blot analysis were extracted from stable transgenic tobacco plants, possessing expression cassettes described in FIG. 3B as follows: 1) sig:OKC1-T:GFP, 2) OKC1:GFP, 3) OS1C1:GFP. Protein lysate extracted from wild type tobacco leave tissues was used as a negative control. 50 ug of protein lysate was loaded into each sample well. Recombinant protein expression was recognized by antibody against the poly-histidine epitope. The poly-epitope control used in western blots, includes GFP, 6×His, FLAG and Myc epitopes. The poly-epitope control has an expected size of 90 kDa and the gel well was loaded with 420 ng of control protein in each blot. The transgenic tobacco plants 003 and 007 having OKC1:GFP and plant 004 having OS1C1:GFP were confirmed with expression of recombinant κ-casein and recombinant α-S1 casein, respectively, as shown in FIGS. 16A and 16B.

### Example 6. Molecular Detection of Transient Expression of Milk Proteins in Soybean (G. max) and Lima Bean (P. lunatus) Embryogenic Callus

Recombinant transgene constructs illustrated in FIGS. 3A and 3B were transformed into embryogenic callus cultures of Soybean and Lima bean using Biolistic bombardment transformation as described in Finer and McMullen, (1991), In Vitro Cell and Develop Biol—Plant 27:175-182).

Green fluorescent protein (GFP) is used extensively as a reporter protein to monitor cellular processes, including intracellular protein trafficking and secretion. It has been noted that GFP oligomerizes in the secretory pathway of endocrine cells, which indicates that oligomerization of GFP and its potential role in GFP transport (Jain et al, 2001; Sanpp El et al, 2003).

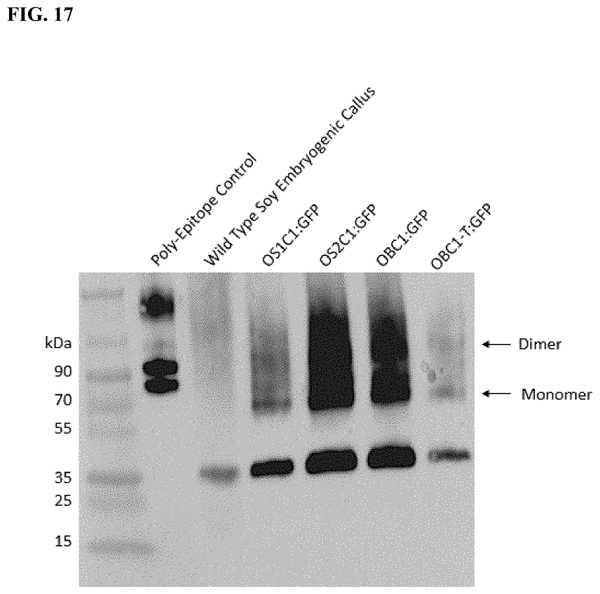

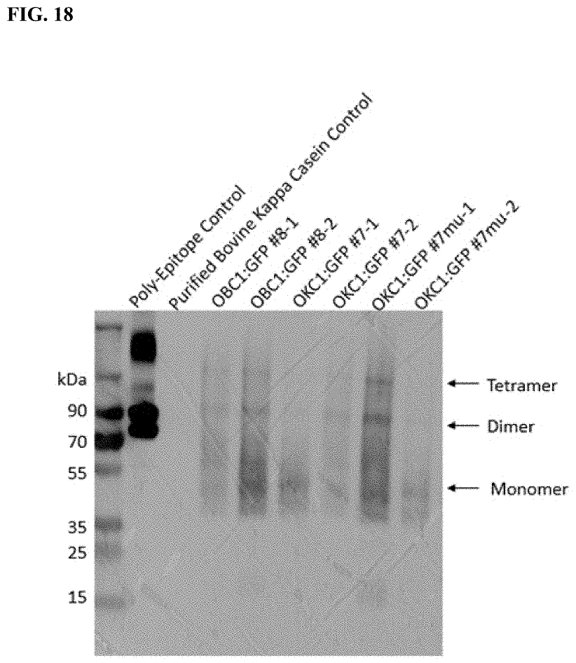

To test transient expression of recombinant milk proteins in Soybean and Lima bean embryogenic calli, western blot analyses were carried out using anti-His antibody. Frozen lyophilized embryogenic callus tissue was finely ground with mortar and pestle and solubilized in PBS (137 mM NaCl; 2.7 mM KCl; 4.3 mM Na2HPO4; 1.47 mM KH2PO4). Soluble lysate was enriched using Ni-NTA affinity chromatography (ThermoFisher Scientific, HisPur Ni-NTA Spin Column) and elution samples were analyzed by SDS PAGE. FIG. 17 shows expression of recombinant α-S1 casein, α-S2 casein, truncated and full-length β-casein proteins. Also, the results indicate that the GFP-fused recombinant casein proteins in FIG. 17 adopt monomeric and dimeric complexes in embryogenic callus tissues. Monomeric and dimeric signal in western blots after transfer from SDS-PAGE was observed under reducing conditions. Similarly, FIG. 18 shows expression of recombinant β-casein and κ-casein proteins with monomeric, dimeric, and even tetrameric complexes in embryogenic callus tissues under reducing conditions. Lysate containing 50 ug of protein was loaded into each sample well. Recombinant protein expression was recognized by antibody against the poly-histidine epitope. The poly-epitope control used in western blots, includes GFP, 6×His, FLAG and Myc epitopes. The poly-epitope control has an expected size of 90 kDa and the gel well was loaded with 420 ng of control protein in each blot.

### Example 7. Visual Detection of Transient and Stable Expression of Milk Proteins in Soybean (G. max) and Lima Bean (P. lunatus) Embryogenic Callus

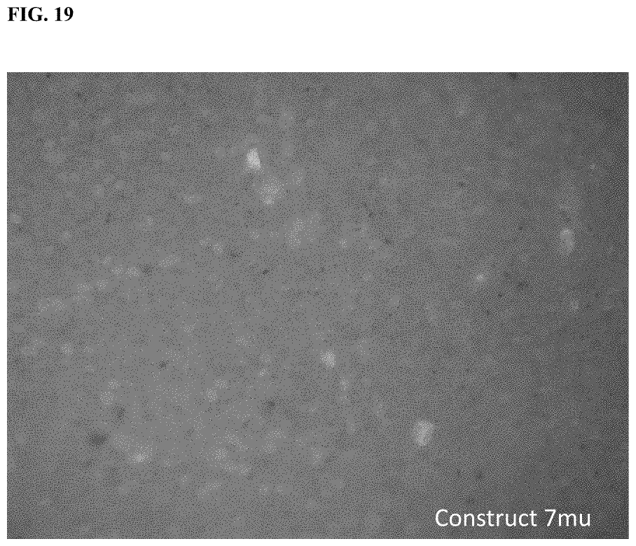

The immature embryogenic lima bean callus tissues as described in FIG. 18. have transient expression of OKC1:GFP:6×His under the control of the GmSM8-1 promoter. To further confirm expression of the recombinant κ-casein fused with GFP protein, the presence of GFP expression was visualized under blue light. Florescent expression of recombinant κ-casein protein under the control of the GmSM8-1 promoter in embryogenic soybean callus tissues was detected as illustrated in FIG. 19. The embryogenic lima bean calli having transgene construct of OKC1:GFP:6×His #7mu successfully express GFP-fused κ-casein protein.

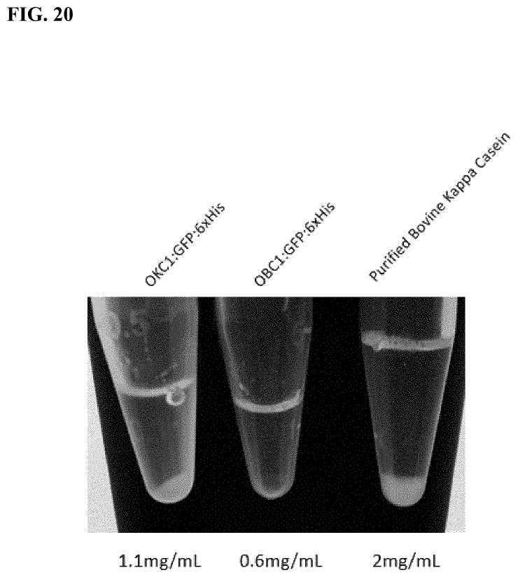

Also, recombinant κ-casein and β-casein proteins were purified from Lima and Soy bean embryogenic callus tissues. FIG. 20 illustrates milky eluant resulting from purification of recombinant milk proteins, OKC1:GFP:6×His and OBC1:GFP:6×His purified from Lima and Soy bean embryogenic callus tissues. The purified recombinant proteins resulted in milky eluants at a concentration of about 1.1 mg/ml of κ-casein from calli expressing OKC1:GFP:6×His and 0.6 mg/ml of β-casein from calli expressing OBC1:GFP:6×His, which are comparable to a solution of control protein at 2.0 mg/ml of the purified bovine κ-casein (Sigma-Aldrich). Purification was achieved by grinding 0.5 g of lyophilized frozen lima bean transformed embryogenic callus and solubilizing in cold PBS buffer (137 mM NaCl; 2.7 mM KCl; 4.3 mM Na2HPO4; 1.47 mM KH2PO4). The soluble sample was enriched using a Ni-NTA affinity column (ThermoFisher Scientific, HisPur Ni-NTA Spin Column). Elution resulted in ˜100 uL of milky solution. This result indicates that not only the transgenic plants expressing recombinant milk proteins, but also embryogenic callus and/or somatic embryo expressing recombinant milk proteins can produce milk proteins for the purpose of food industrial, non-food industrial, pharmaceutical, and commercial uses described in this disclosure.

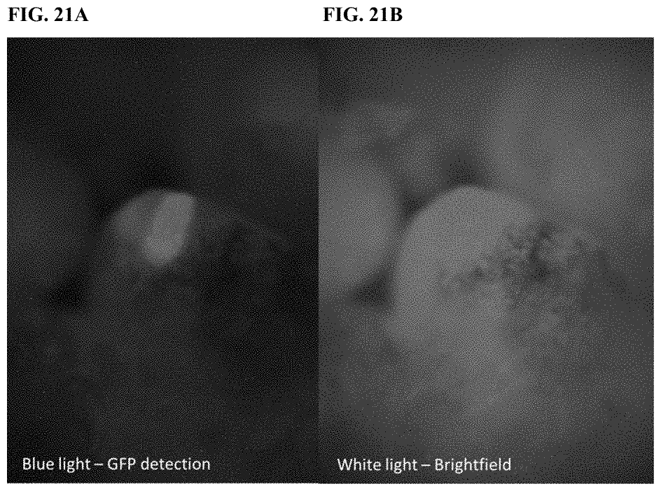

To generate stable transgenic soybean plants for expression of milk proteins, plasmid DNA containing three constructs (AR-Pro3:OKC1:GFP; gmSM8-1:OKC1:GFP; gmSM8-1:OBC1:GFP) were introduced into embryogenic cultures respectively. Biolistic bombardment transformation was performed into embryogenic soybean variety Jack, as described in Finer and McMullen, (1991), In Vitro Cell and Develop Biol—Plant 27:175-182). Using hygromycin as a selectable marker, the resistant embryogenic events were recovered and visually screened for the presence of GFP to confirm the fusion protein expression (FIG. 21A).

FIG. 21A-21B shows florescence expression of recombinant κ-casein protein under the control of the GmSM8-1 promoter in embryogenic soybean callus tissues. Clones are placed on embryo development medium (e.g. M6AC) for maturation and development of seed-like traits. The leaves of the primary transformants (T₀) regenerated from calli are fluorescent under blue light indicating high levels of GFP expression. The seed from the self-fertilized T₀ plants are viable, and the resulting T₁ seedlings harboring the transgene can be tested to detect fluorescence.

### Example 8. Molecular Read-Out for Expression of Milk Proteins in Tobacco, Soybean, Lima Bean, and Rice

To quantify transgene expression level of the provided target proteins including α-S1, α-S2, β-, κ-casein proteins, α-lactalbumin, β-lactoglobulin, and lysozyme, quantificational analysis using GUS enzyme activity analysis kit is performed according to experimental procedure as described in (www.markergene.com/gus-reporter-gene-activity-detection-kit.html). Also, Trizol and RNA purification kit is used to extract RNA from leave, callus, embryo and/or seed tissues and transgene expression level is tested by RT-PCR and real-time quantitative PCR techniques. RT-PCR results can be used as a molecular read-out of milk protein expression.

Expression pattern and level of transcripts corresponding to the provided target proteins including α-S1, α-S2, β-, κ-casein proteins, α-lactalbumin, β-lactoglobulin, and lysozyme are observed and measured.

### Example 9. Expression and Production of Milk Proteins in Arabidopsis and Duckweed

Recombinant DNA constructs with transgenes, for example, such constructs described in FIGS. 2A-3C are transformed into Arabidopsis using Arabidopsis transformation protocol well known in the art. GUS activity and GFP expression are detected from transgenic Arabidopsis plants into whose genome each of various chimeric transgenes encoding bovine milk proteins provided herein is stably integrated. Also, GUS and/or GFP-fusion milk proteins are observed from western blot analyses. Expression corresponding to the demonstrated target proteins including α-S1, α-S2, β-, κ-casein proteins, α-lactalbumin, β-lactoglobulin, and lysozyme are observed and measured using experimental methods described in Examples 5 and 6. Modification in protocols may be applied according to plant types and conditions.

The contents of the milky eluent from the expressed milk protein in Arabidopsis plants are measured. RT-PCR can be performed to measure a molecular read-out of milk protein expression in the transgenic Arabidopsis plants.

Recombinant DNA constructs with transgenes, for example, such constructs described in FIGS. 2A-3C are transformed into Duckweed using duckweed transformation protocol well known in the art. GUS activity and GFP expression are detected from transgenic duckweed plants into whose genome each of various chimeric transgenes encoding bovine milk proteins provided herein is transiently or stably integrated. Also, GUS and/or GFP-fusion milk proteins are observed from western blot analyses. Expression corresponding to the demonstrated target proteins including α-S1, α-S2, β-, κ-casein proteins, α-lactalbumin, β-lactoglobulin, and lysozyme are observed and measured using experimental methods described in Examples 5 and 6. Modification in protocols may be applied according to plant types and conditions.

The contents of the milky eluent from the expressed milk protein in transgenic duckweed plants are measured. RT-PCR can be performed to measure a molecular read-out of milk protein expression in transgenic duckweed plants.

### Example 10. Expression and Production of Milk Proteins in Somatic Embryos and/or Mature Embryos

Recombinant DNA constructs with transgenes, for example, such constructs described in FIGS. 2A-3C are transformed into somatic embryos and/or mature embryos using microprojectile bombardment, such as particle acceleration or biolistic bombardment and/or Agrobacterium-mediated transformation. Somatic embryos can be prepared from soybean, lima bean, tobacco and/or rice. Milk proteins including α-S1, α-S2, β-, κ-casein proteins, α-lactalbumin, β-lactoglobulin, and lysozyme, are detected in said somatic embryos using GUS staining and/or GFP detection test. Also, GUS and/or GFP-fusion milk proteins are observed from western blot analyses. Expression corresponding to the demonstrated target proteins including α-S1, α-S2, β-, κ-casein proteins, α-lactalbumin, β-lactoglobulin, and lysozyme are observed and measured using experimental methods described in Examples 5 and 6. Modification in protocols may be applied according to plant types and conditions. Transgene expression level is also tested by RT-PCR and real-time quantitative PCR techniques. RT-PCR results can be used as a molecular read-out of milk protein expression. Furthermore, the contents of the milky eluent of the expressed milk proteins in somatic embryos and/or mature embryos are measured.

Although the foregoing disclosure has been described in some detail by way of illustration and examples, which are for purposes of clarity of understanding, it will be apparent to those skilled in the art that certain changes and modifications may be practiced without departing from the spirit and scope of the disclosure, which is delineated in the appended claims. Therefore, the description should not be construed as limiting the scope of the disclosure.

### NUMBERED EMBODIMENTS OF THE DISCLOSURE

Notwithstanding the appended claims, the disclosure sets forth the following numbered embodiments:

### Transgenic Plants

1. A transgenic plant comprising a recombinant DNA construct, said construct comprising
(i) a promoter,
(ii) a nucleic acid sequence encoding a bovine milk protein and/or a functional fragment thereof, which is operably linked to said promoter, and
(iii) a termination sequence;
   wherein the bovine milk protein and/or the functional fragment thereof is expressed in the transgenic plant and/or a part thereof; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.
2. The transgenic plant of claim 1, wherein the plant is a dicot plant selected from the group consisting of soybean, lima bean, Arabidopsis, and tobacco.
3. The transgenic plant of claim 1, wherein the plant is a monocot plant selected from the group consisting of duckweed, rice, maize, oat, barley, and wheat.
4. The transgenic plant of any one of claims 1-3, wherein the promoter is selected from a Cauliflower mosaic virus (CaMV) 35S promoter, a plant constitutive promoter, and a plant tissue-specific promoter.
5. The transgenic plant of any one of claims 1-4, wherein the plant constitutive promoter comprises an nucleic acid having at least 90% sequence identity to SEQ ID No:46, SEQ ID No:47, SEQ ID No:48, and SEQ ID No:49.
6. The transgenic plant of any one of claims 1-4, wherein the plant tissue-specific promoter comprises an nucleic acid having at least 90% sequence identity to SEQ ID No:28, SEQ ID No:30, SEQ ID No:32, SEQ ID No:34, SEQ ID No:36, SEQ ID No:38, SEQ ID No:40, SEQ ID No:42 and SEQ ID No:44.
7. The transgenic plant of any one of claims 1-6, wherein the nucleic acid sequence encoding κ-casein and/or the functional fragment thereof is codon-optimized.
8. The transgenic plant of any one of claims 1-6, wherein the nucleic acid sequence encoding β-casein and/or the functional fragment thereof is codon-optimized.
9. The transgenic plant of any one of claims 1-6, wherein the nucleic acid sequence encoding α-S1 casein and/or the functional fragment thereof is codon-optimized.
10. The transgenic plant of any one of claims 1-6, wherein the nucleic acid sequence encoding α-S2 casein and/or the functional fragment thereof is codon-optimized.
11. The transgenic plant of any one of claims 1-6, wherein the nucleic acid sequence encoding α-lactalbumin and/or the functional fragment thereof is codon-optimized.
12. The transgenic plant of any one of claims 1-6, wherein the nucleic acid sequence encoding β-lactoglobulin and/or the functional fragment thereof is codon-optimized.
13. The transgenic plant of any one of claims 1-6, wherein the nucleic acid sequence encoding lysozyme and/or the functional fragment thereof is codon-optimized.
14. The transgenic plant of any one of claims 1-7, wherein the nucleic acid sequence encodes κ-casein protein and/or the functional fragment thereof, having at least 90% sequence identity to SEQ ID No:5.
15. The transgenic plant of any one of claims 1-6 and 8, wherein the nucleic acid sequence encoding β-casein and/or the functional fragment thereof, having at least 90% sequence identity to SEQ ID No:7.
16. The transgenic plant of any one of claims 1-6 and 9, wherein the nucleic acid sequence encoding α-S1 casein and/or the functional fragment thereof, having at least 90% sequence identity to SEQ ID No:11.
17. The transgenic plant of any one of claims 1-6 and 10, wherein the nucleic acid sequence encoding α-S2 casein and/or the functional fragment thereof, having at least 90% sequence identity to SEQ ID No:12.
18. The transgenic plant of any one of claims 1-6 and 11, wherein the nucleic acid sequence encoding α-lactalbumin and/or the functional fragment, having at least 90% sequence identity to SEQ ID No:22.
19. The transgenic plant of any one of claims 1-6 and 12, wherein the nucleic acid sequence encoding β-lactoglobulin and/or the functional fragment thereof, having at least 90% sequence identity to SEQ ID No:23.
20. The transgenic plant of any one of claims 1-6 and 13, wherein the nucleic acid sequence encoding lysozyme and/or the functional fragment, having at least 90% sequence identity to SEQ ID No:24.
21. The transgenic plant of any one of claims 1-20, wherein the termination sequence is a NOS terminator.
22. The transgenic plant of any one of claims 1-21, wherein the bovine milk protein comprises α-S1 casein, α-S2 casein, β-casein, κ-casein, α-lactalbumin, β-lactoglobulin, and lysozyme.
23. The transgenic plant of any one of claims 1-22, wherein the bovine milk protein further comprises proteolytic product of α-S1 casein, α-S2 casein, β-casein, κ-casein, α-lactalbumin, β-lactoglobulin, and lysozyme.
24. The transgenic plant of any one of claims 1-23, wherein the bovine milk protein further comprises peptides produced by proteolysis of α-S1 casein, α-S2 casein, β-casein κ-casein, lactalbumin, β-lactoglobulin, and lysozyme.
25. A method of producing said transgenic plant of any one of claims 1-24, said method comprising the steps of:

(a) introducing at least one expression cassette capable of expressing a bovine milk protein into a plant, a part thereof, or a cell thereof,

(b) obtaining the transgenic plant, the part thereof, or the cell thereof, which stably expresses the bovine milk protein

(c) cultivating the transgenic plant, the part thereof, or the cell thereof,

(d) harvesting the transgenic plant, the part thereof, or the cell thereof.
26. A method of producing a bovine milk protein from said transgenic plant of any one of claims 1-24, said method comprising the steps of:

(a) extracting the bovine milk protein from the transgenic plant, the part thereof, or the cell thereof, and

(b) purifying the bovine milk protein from the transgenic plant, the part thereof, or the cell thereof;

wherein the bovine milk protein comprises α-S1 casein, α-S2 casein, β-casein, κ-casein, α-lactalbumin, β-lactoglobulin, and lysozyme; wherein the bovine milk protein further comprises proteolytic product of α-S1 casein, α-S2 casein, β-casein, κ-casein, α-lactalbumin, β-lactoglobulin, and lysozyme; and wherein the bovine milk protein further comprises peptides produced by proteolysis of α-S1 casein, α-S2 casein, β-casein, κ-casein, α-lactalbumin, β-lactoglobulin, and lysozyme.

### Soybean

1. A transgenic soybean plant comprising a recombinant DNA construct, said construct comprising
(i) a promoter,
(ii) a nucleic acid sequence encoding a bovine milk protein and/or a functional fragment thereof, which is operably linked to said promoter, and
(iii) a termination sequence;
   wherein the bovine milk protein and/or the functional fragment thereof is expressed in the transgenic soybean plant and/or a part thereof; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.
2. The transgenic soybean plant of claim 1, wherein the promoter is selected from a Cauliflower mosaic virus (CaMV) 35S promoter, a plant constitutive promoter, and a plant tissue-specific promoter.
3. The transgenic soybean plant of any one of claims 1-2, wherein the plant constitutive promoter comprises an nucleic acid having at least 90% sequence identity to SEQ ID No:46, SEQ ID No:47, SEQ ID No:48, and SEQ ID No:49.
4. The transgenic soybean plant of any one of claims 1-3, wherein the plant tissue-specific promoter comprises an nucleic acid having at least 90% sequence identity to SEQ ID No:28, SEQ ID No:30, SEQ ID No:32, SEQ ID No:34, SEQ ID No:36, SEQ ID No:38, SEQ ID No:40, SEQ ID No:42 and SEQ ID No:44.
5. The transgenic soybean plant of any one of claims 1-4, wherein the nucleic acid sequence encoding κ-casein and/or the functional fragment thereof is codon-optimized.
6. The transgenic soybean plant of any one of claims 1-4, wherein the nucleic acid sequence encoding κ-casein and/or the functional fragment thereof is codon-optimized.
7. The transgenic soybean plant of any one of claims 1-4, wherein the nucleic acid sequence encoding β-casein and/or the functional fragment thereof is codon-optimized.
8. The transgenic soybean plant of any one of claims 1-4, wherein the nucleic acid sequence encoding α-S1 casein and/or the functional fragment thereof is codon-optimized.
9. The transgenic soybean plant of any one of claims 1-4, wherein the nucleic acid sequence encoding α-S2 casein and/or the functional fragment thereof is codon-optimized.
10. The transgenic soybean plant of any one of claims 1-4, wherein the nucleic acid sequence encoding α-lactalbumin and/or the functional fragment thereof is codon-optimized.
11. The transgenic soybean plant of any one of claims 1-4, wherein the nucleic acid sequence encoding β-lactoglobulin and/or the functional fragment thereof is codon-optimized.
12. The transgenic soybean plant of any one of claims 1-4, wherein the nucleic acid sequence encoding lysozyme and/or the functional fragment thereof is codon-optimized.
13. The transgenic soybean plant of any one of claims 1-5, wherein the nucleic acid sequence encodes κ-casein protein and/or the functional fragment thereof, having at least 90% sequence identity to SEQ ID No:5.
14. The transgenic soybean plant of any one of claims 1-4 and 6, wherein the nucleic acid sequence encoding β-casein and/or the functional fragment thereof, having at least 90% sequence identity to SEQ ID No:7.
15. The transgenic soybean plant of any one of claims 1-4 and 7, wherein the nucleic acid sequence encoding α-S1 casein and/or the functional fragment thereof, having at least 90% sequence identity to SEQ ID No:11.
16. The transgenic soybean plant of any one of claims 1-4 and 8, wherein the nucleic acid sequence encoding α-S2 casein and/or the functional fragment thereof, having at least 90% sequence identity to SEQ ID No:12.
17. The transgenic soybean plant of any one of claims 1-4 and 9, wherein the nucleic acid sequence encoding α-lactalbumin and/or the functional fragment, having at least 90% sequence identity to SEQ ID No:22.
18. The transgenic soybean plant of any one of claims 1-4 and 10, wherein the nucleic acid sequence encoding β-lactoglobulin and/or the functional fragment thereof, having at least 90% sequence identity to SEQ ID No:23.
19. The transgenic soybean plant of any one of claims 1-4 and 11, wherein the nucleic acid sequence encoding lysozyme and/or the functional fragment, having at least 90% sequence identity to SEQ ID No:24.
20. The transgenic soybean plant of any one of claims 1-18, wherein the termination sequence is a NOS terminator.
21. The transgenic soybean plant of any one of claims 1-19, wherein the bovine milk protein comprises α-S1 casein, α-S2 casein, β-casein, κ-casein, α-lactalbumin, β-lactoglobulin, and lysozyme.
22. The transgenic soybean plant of any one of claims 1-20, wherein the bovine milk protein further comprises proteolytic product of α-S1 casein, α-S2 casein, β-casein, κ-casein, α-lactalbumin, β-lactoglobulin, and lysozyme.
23. The transgenic soybean plant of any one of claims 1-21, wherein the bovine milk protein further comprises peptides produced by proteolysis of α-S1 casein, α-S2 casein, β-casein κ-casein, lactalbumin, β-lactoglobulin, and lysozyme.
24. A method of producing said transgenic soybean plant of any one of claims 1-22, said method comprising the steps of:

(a) introducing at least one expression cassette capable of expressing a bovine milk protein into a soybean plant, a part thereof, or a cell thereof,

(b) obtaining the transgenic soybean plant, the part thereof, or the cell thereof, which stably expresses the bovine milk protein

(c) cultivating the transgenic soybean plant, the part thereof, or the cell thereof,

(d) harvesting the transgenic soybean plant, the part thereof, or the cell thereof.
25. A method of producing a bovine milk protein from said transgenic soybean plant of any one of claims 1-22, said method comprising the steps of:

(a) extracting the bovine milk protein from the transgenic soybean plant, the part thereof, or the cell thereof, and

(b) purifying the bovine milk protein from the transgenic soybean plant, the part thereof, or the cell thereof;

wherein the bovine milk protein comprises α-S1 casein, α-S2 casein, β-casein, κ-casein, α-lactalbumin, β-lactoglobulin, and lysozyme; wherein the bovine milk protein further comprises proteolytic product of α-S1 casein, α-S2 casein, β-casein, κ-casein, α-lactalbumin, β-lactoglobulin, and lysozyme; and wherein the bovine milk protein further comprises peptides produced by proteolysis of α-S1 casein, α-S2 casein, β-casein, κ-casein, α-lactalbumin, β-lactoglobulin, and lysozyme.

### Lima Bean

1. A transgenic lima bean plant comprising a recombinant DNA construct, said construct comprising
(i) a promoter,
(ii) a nucleic acid sequence encoding a bovine milk protein and/or a functional fragment thereof, which is operably linked to said promoter, and
(iii) a termination sequence;
   wherein the bovine milk protein and/or the functional fragment thereof is expressed in the transgenic lima bean plant and/or a part thereof; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.
2. The transgenic lima bean plant of claim 1, wherein the promoter is selected from a Cauliflower mosaic virus (CaMV) 35S promoter, a plant constitutive promoter, and a plant tissue-specific promoter.
3. The transgenic lima bean plant of any one of claims 1-2, wherein the plant constitutive promoter comprises an nucleic acid having at least 90% sequence identity to SEQ ID No:46, SEQ ID No:47, SEQ ID No:48, and SEQ ID No:49.
4. The transgenic lima bean plant of any one of claims 1-3, wherein the plant tissue-specific promoter comprises an nucleic acid having at least 90% sequence identity to SEQ ID No:28, SEQ ID No:30, SEQ ID No:32, SEQ ID No:34, SEQ ID No:36, SEQ ID No:38, SEQ ID No:40, SEQ ID No:42 and SEQ ID No:44.
5. The transgenic lima bean plant of any one of claims 1-4, wherein the nucleic acid sequence encoding κ-casein and/or the functional fragment thereof is codon-optimized.
6. The transgenic lima bean plant of any one of claims 1-4, wherein the nucleic acid sequence encoding β-casein and/or the functional fragment thereof is codon-optimized.
7. The transgenic lima bean plant of any one of claims 1-4, wherein the nucleic acid sequence encoding α-S1 casein and/or the functional fragment thereof is codon-optimized.
8. The transgenic lima bean plant of any one of claims 1-4, wherein the nucleic acid sequence encoding α-S2 casein and/or the functional fragment thereof is codon-optimized.
9. The transgenic lima bean plant of any one of claims 1-4, wherein the nucleic acid sequence encoding α-lactalbumin and/or the functional fragment thereof is codon-optimized.
10. The transgenic lima bean plant of any one of claims 1-4, wherein the nucleic acid sequence encoding β-lactoglobulin and/or the functional fragment thereof is codon-optimized.
11. The transgenic lima bean plant of any one of claims 1-4, wherein the nucleic acid sequence encoding lysozyme and/or the functional fragment thereof is codon-optimized.
12. The transgenic lima bean plant of any one of claims 1-5, wherein the nucleic acid sequence encodes κ-casein protein and/or the functional fragment thereof, having at least 90% sequence identity to SEQ ID No:5.
13. The transgenic lima bean plant of any one of claims 1-4 and 6, wherein the nucleic acid sequence encoding β-casein and/or the functional fragment thereof, having at least 90% sequence identity to SEQ ID No:7.
14. The transgenic lima bean plant of any one of claims 1-4 and 7, wherein the nucleic acid sequence encoding α-S1 casein and/or the functional fragment thereof, having at least 90% sequence identity to SEQ ID No:11.
15. The transgenic lima bean plant of any one of claims 1-4 and 8, wherein the nucleic acid sequence encoding α-S2 casein and/or the functional fragment thereof, having at least 90% sequence identity to SEQ ID No:12.
16. The transgenic lima bean plant of any one of claims 1-4 and 9, wherein the nucleic acid sequence encoding α-lactalbumin and/or the functional fragment, having at least 90% sequence identity to SEQ ID No:22.
17. The transgenic lima bean plant of any one of claims 1-4 and 10, wherein the nucleic acid sequence encoding β-lactoglobulin and/or the functional fragment thereof, having at least 90% sequence identity to SEQ ID No:23. 18. The transgenic lima bean plant of any one of claims 1-4 and 11, wherein the nucleic acid sequence encoding lysozyme and/or the functional fragment, having at least 90% sequence identity to SEQ ID No:24.
19. The transgenic lima bean plant of any one of claims 1-18, wherein the termination sequence is a NOS terminator.
20. The transgenic lima bean plant of any one of claims 1-19, wherein the bovine milk protein comprises α-S1 casein, α-S2 casein, β-casein, κ-casein, α-lactalbumin, β-lactoglobulin, and lysozyme.
21. The transgenic lima bean plant of any one of claims 1-20, wherein the bovine milk protein further comprises proteolytic product of α-S1 casein, α-S2 casein, β-casein, κ-casein, α-lactalbumin, β-lactoglobulin, and lysozyme.
22. The transgenic lima bean plant of any one of claims 1-21, wherein the bovine milk protein further comprises peptides produced by proteolysis of α-S1 casein, α-S2 casein, β-casein κ-casein, lactalbumin, β-lactoglobulin, and lysozyme.
23. A method of producing said transgenic lima bean plant of any one of claims 1-22, said method comprising the steps of:

(a) introducing at least one expression cassette capable of expressing a bovine milk protein into a lima bean plant, a part thereof, or a cell thereof,

(b) obtaining the transgenic lima bean plant, the part thereof, or the cell thereof, which stably expresses the bovine milk protein

(c) cultivating the transgenic lima bean plant, the part thereof, or the cell thereof,

(d) harvesting the transgenic lima bean plant, the part thereof, or the cell thereof.
24. A method of producing a bovine milk protein from said transgenic lima bean plant of any one of claims 1-22, said method comprising the steps of:

(a) extracting the bovine milk protein from the transgenic lima bean plant, the part thereof, or the cell thereof, and

(b) purifying the bovine milk protein from the transgenic lima bean plant, the part thereof, or the cell thereof;

wherein the bovine milk protein comprises α-S1 casein, α-S2 casein, β-casein, κ-casein, α-lactalbumin, β-lactoglobulin, and lysozyme; wherein the bovine milk protein further comprises proteolytic product of α-S1 casein, α-S2 casein, β-casein, κ-casein, α-lactalbumin, β-lactoglobulin, and lysozyme; and wherein the bovine milk protein further comprises peptides produced by proteolysis of α-S1 casein, α-S2 casein, β-casein, κ-casein, α-lactalbumin, β-lactoglobulin, and lysozyme.

### Tobacco

1. A transgenic tobacco plant comprising a recombinant DNA construct, said construct comprising
(i) a promoter,
(ii) a nucleic acid sequence encoding a bovine milk protein and/or a functional fragment thereof, which is operably linked to said promoter, and
(iii) a termination sequence;
   wherein the bovine milk protein and/or the functional fragment thereof is expressed in the transgenic tobacco plant and/or a part thereof; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.
2. The transgenic tobacco plant of claim 1, wherein the promoter is selected from a Cauliflower mosaic virus (CaMV) 35S promoter, a plant constitutive promoter, and a plant tissue-specific promoter.
3. The transgenic tobacco plant of any one of claims 1-2, wherein the plant constitutive promoter comprises an nucleic acid having at least 90% sequence identity to SEQ ID No:46, SEQ ID No:47, SEQ ID No:48, and SEQ ID No:49.
4. The transgenic tobacco plant of any one of claims 1-3, wherein the plant tissue-specific promoter comprises an nucleic acid having at least 90% sequence identity to SEQ ID No:28, SEQ ID No:30, SEQ ID No:32, SEQ ID No:34, SEQ ID No:36, SEQ ID No:38, SEQ ID No:40, SEQ ID No:42 and SEQ ID No:44.
5. The transgenic tobacco plant of any one of claims 1-4, wherein the nucleic acid sequence encoding κ-casein and/or the functional fragment thereof is codon-optimized.
6. The transgenic tobacco plant of any one of claims 1-4, wherein the nucleic acid sequence encoding β-casein and/or the functional fragment thereof is codon-optimized.
7. The transgenic tobacco plant of any one of claims 1-4, wherein the nucleic acid sequence encoding α-S1 casein and/or the functional fragment thereof is codon-optimized.
8. The transgenic tobacco plant of any one of claims 1-4, wherein the nucleic acid sequence encoding α-S2 casein and/or the functional fragment thereof is codon-optimized.
9. The transgenic tobacco plant of any one of claims 1-4, wherein the nucleic acid sequence encoding α-lactalbumin and/or the functional fragment thereof is codon-optimized.
10. The transgenic tobacco plant of any one of claims 1-4, wherein the nucleic acid sequence encoding β-lactoglobulin and/or the functional fragment thereof is codon-optimized.
11. The transgenic tobacco plant of any one of claims 1-4, wherein the nucleic acid sequence encoding lysozyme and/or the functional fragment thereof is codon-optimized.
12. The transgenic tobacco plant of any one of claims 1-5, wherein the nucleic acid sequence encodes κ-casein protein and/or the functional fragment thereof, having at least 90% sequence identity to SEQ ID No:5.
13. The transgenic tobacco plant of any one of claims 1-4 and 6, wherein the nucleic acid sequence encoding β-casein and/or the functional fragment thereof, having at least 90% sequence identity to SEQ ID No:7.
14. The transgenic tobacco plant of any one of claims 1-4 and 7, wherein the nucleic acid sequence encoding α-S1 casein and/or the functional fragment thereof, having at least 90% sequence identity to SEQ ID No:11.
15. The transgenic tobacco plant of any one of claims 1-4 and 8, wherein the nucleic acid sequence encoding α-S2 casein and/or the functional fragment thereof, having at least 90% sequence identity to SEQ ID No:12.
16. The transgenic tobacco plant of any one of claims 1-4 and 9, wherein the nucleic acid sequence encoding α-lactalbumin and/or the functional fragment, having at least 90% sequence identity to SEQ ID No:22.
17. The transgenic tobacco plant of any one of claims 1-4 and 10, wherein the nucleic acid sequence encoding β-lactoglobulin and/or the functional fragment thereof, having at least 90% sequence identity to SEQ ID No:23.
18. The transgenic tobacco plant of any one of claims 1-4 and 11, wherein the nucleic acid sequence encoding lysozyme and/or the functional fragment, having at least 90% sequence identity to SEQ ID No:24.
19. The transgenic tobacco plant of any one of claims 1-18, wherein the termination sequence is a NOS terminator.
20. The transgenic tobacco plant of any one of claims 1-19, wherein the bovine milk protein comprises α-S1 casein, α-S2 casein, β-casein, κ-casein, α-lactalbumin, β-lactoglobulin, and lysozyme.
21. The transgenic tobacco plant of any one of claims 1-20, wherein the bovine milk protein further comprises proteolytic product of α-S1 casein, α-S2 casein, β-casein, κ-casein, α-lactalbumin, β-lactoglobulin, and lysozyme.
22. The transgenic tobacco plant of any one of claims 1-21, wherein the bovine milk protein further comprises peptides produced by proteolysis of α-S1 casein, α-S2 casein, β-casein κ-casein, lactalbumin, β-lactoglobulin, and lysozyme.
23. A method of producing said transgenic tobacco plant of any one of claims 1-22, said method comprising the steps of:

(a) introducing at least one expression cassette capable of expressing a bovine milk protein into a tobacco plant, a part thereof, or a cell thereof,

(b) obtaining the transgenic tobacco plant, the part thereof, or the cell thereof, which stably expresses the bovine milk protein

(c) cultivating the transgenic tobacco plant, the part thereof, or the cell thereof,

(d) harvesting the transgenic tobacco plant, the part thereof, or the cell thereof.
24. A method of producing a bovine milk protein from said transgenic tobacco plant of any one of claims 1-22, said method comprising the steps of:

(a) extracting the bovine milk protein from the transgenic tobacco plant, the part thereof, or the cell thereof, and

(b) purifying the bovine milk protein from the transgenic tobacco plant, the part thereof, or the cell thereof;

wherein the bovine milk protein comprises α-S1 casein, α-S2 casein, β-casein, κ-casein, α-lactalbumin, β-lactoglobulin, and lysozyme; wherein the bovine milk protein further comprises proteolytic product of α-S1 casein, α-S2 casein, β-casein, κ-casein, α-lactalbumin, β-lactoglobulin, and lysozyme; and wherein the bovine milk protein further comprises peptides produced by proteolysis of α-S1 casein, α-S2 casein, β-casein, κ-casein, α-lactalbumin, β-lactoglobulin, and lysozyme.

### Arabidopsis

1. A transgenic arabidopsis plant comprising a recombinant DNA construct, said construct comprising
(i) a promoter,
(ii) a nucleic acid sequence encoding a bovine milk protein and/or a functional fragment thereof, which is operably linked to said promoter, and
(iii) a termination sequence;
   wherein the bovine milk protein and/or the functional fragment thereof is expressed in the transgenic arabidopsis plant and/or a part thereof; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.
2. The transgenic arabidopsis plant of claim 1, wherein the promoter is selected from a Cauliflower mosaic virus (CaMV) 35S promoter, a plant constitutive promoter, and a plant tissue-specific promoter.
3. The transgenic arabidopsis plant of any one of claims 1-2, wherein the plant constitutive promoter comprises an nucleic acid having at least 90% sequence identity to SEQ ID No:46, SEQ ID No:47, SEQ ID No:48, and SEQ ID No:49.
4. The transgenic arabidopsis plant of any one of claims 1-3, wherein the plant tissue-specific promoter comprises an nucleic acid having at least 90% sequence identity to SEQ ID No:28, SEQ ID No:30, SEQ ID No:32, SEQ ID No:34, SEQ ID No:36, SEQ ID No:38, SEQ ID No:40, SEQ ID No:42 and SEQ ID No:44.
5. The transgenic arabidopsis plant of any one of claims 1-4, wherein the nucleic acid sequence encoding κ-casein and/or the functional fragment thereof is codon-optimized.
6. The transgenic arabidopsis plant of any one of claims 1-4, wherein the nucleic acid sequence encoding β-casein and/or the functional fragment thereof is codon-optimized.
7. The transgenic arabidopsis plant of any one of claims 1-4, wherein the nucleic acid sequence encoding α-S1 casein and/or the functional fragment thereof is codon-optimized.
8. The transgenic arabidopsis plant of any one of claims 1-4, wherein the nucleic acid sequence encoding α-S2 casein and/or the functional fragment thereof is codon-optimized.
9. The transgenic arabidopsis plant of any one of claims 1-4, wherein the nucleic acid sequence encoding α-lactalbumin and/or the functional fragment thereof is codon-optimized.
10. The transgenic arabidopsis plant of any one of claims 1-4, wherein the nucleic acid sequence encoding β-lactoglobulin and/or the functional fragment thereof is codon-optimized.
11. The transgenic arabidopsis plant of any one of claims 1-4, wherein the nucleic acid sequence encoding lysozyme and/or the functional fragment thereof is codon-optimized.
12. The transgenic arabidopsis plant of any one of claims 1-5, wherein the nucleic acid sequence encodes κ-casein protein and/or the functional fragment thereof, having at least 90% sequence identity to SEQ ID No:5.
13. The transgenic arabidopsis plant of any one of claims 1-4 and 6, wherein the nucleic acid sequence encoding β-casein and/or the functional fragment thereof, having at least 90% sequence identity to SEQ ID No:7.
14. The transgenic arabidopsis plant of any one of claims 1-4 and 7, wherein the nucleic acid sequence encoding α-S1 casein and/or the functional fragment thereof, having at least 90% sequence identity to SEQ ID No:11.
15. The transgenic arabidopsis plant of any one of claims 1-4 and 8, wherein the nucleic acid sequence encoding α-S2 casein and/or the functional fragment thereof, having at least 90% sequence identity to SEQ ID No:12.
16. The transgenic arabidopsis plant of any one of claims 1-4 and 9, wherein the nucleic acid sequence encoding α-lactalbumin and/or the functional fragment, having at least 90% sequence identity to SEQ ID No:22.
17. The transgenic arabidopsis plant of any one of claims 1-4 and 10, wherein the nucleic acid sequence encoding β-lactoglobulin and/or the functional fragment thereof, having at least 90% sequence identity to SEQ ID No:23.
18. The transgenic arabidopsis plant of any one of claims 1-4 and 11, wherein the nucleic acid sequence encoding lysozyme and/or the functional fragment, having at least 90% sequence identity to SEQ ID No:24.
19. The transgenic arabidopsis plant of any one of claims 1-18, wherein the termination sequence is a NOS terminator.
20. The transgenic arabidopsis plant of any one of claims 1-19, wherein the bovine milk protein comprises α-S1 casein, α-S2 casein, β-casein, κ-casein, α-lactalbumin, β-lactoglobulin, and lysozyme.
21. The transgenic arabidopsis plant of any one of claims 1-20, wherein the bovine milk protein further comprises proteolytic product of α-S1 casein, α-S2 casein, β-casein, κ-casein, α-lactalbumin, β-lactoglobulin, and lysozyme.
22. The transgenic arabidopsis plant of any one of claims 1-21, wherein the bovine milk protein further comprises peptides produced by proteolysis of α-S1 casein, α-S2 casein, β-casein κ-casein, lactalbumin, β-lactoglobulin, and lysozyme.
23. A method of producing said transgenic arabidopsis plant of any one of claims 1-22, said method comprising the steps of:

(a) introducing at least one expression cassette capable of expressing a bovine milk protein into a arabidopsis plant, a part thereof, or a cell thereof,

(b) obtaining the transgenic arabidopsis plant, the part thereof, or the cell thereof, which stably expresses the bovine milk protein

(c) cultivating the transgenic arabidopsis plant, the part thereof, or the cell thereof,

(d) harvesting the transgenic arabidopsis plant, the part thereof, or the cell thereof.
24. A method of producing a bovine milk protein from said transgenic arabidopsis plant of any one of claims 1-22, said method comprising the steps of:

(a) extracting the bovine milk protein from the transgenic arabidopsis plant, the part thereof, or the cell thereof, and

(b) purifying the bovine milk protein from the transgenic arabidopsis plant, the part thereof, or the cell thereof;

wherein the bovine milk protein comprises α-S1 casein, α-S2 casein, β-casein, κ-casein, α-lactalbumin, β-lactoglobulin, and lysozyme; wherein the bovine milk protein further comprises proteolytic product of α-S1 casein, α-S2 casein, β-casein, κ-casein, α-lactalbumin, β-lactoglobulin, and lysozyme; and wherein the bovine milk protein further comprises peptides produced by proteolysis of α-S1 casein, α-S2 casein, β-casein, κ-casein, α-lactalbumin, β-lactoglobulin, and lysozyme.

### Rice

1. A transgenic rice plant comprising a recombinant DNA construct, said construct comprising
(i) a promoter,
(ii) a nucleic acid sequence encoding a bovine milk protein and/or a functional fragment thereof, which is operably linked to said promoter, and
(iii) a termination sequence;
   wherein the bovine milk protein and/or the functional fragment thereof is expressed in the transgenic rice plant and/or a part thereof; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.
2. The transgenic rice plant of claim 1, wherein the promoter is selected from a Cauliflower mosaic virus (CaMV) 35S promoter, a plant constitutive promoter, and a plant tissue-specific promoter.
3. The transgenic rice plant of any one of claims 1-2, wherein the plant constitutive promoter comprises an nucleic acid having at least 90% sequence identity to SEQ ID No:46, SEQ ID No:47, SEQ ID No:48, and SEQ ID No:49.
4. The transgenic rice plant of any one of claims 1-3, wherein the plant tissue-specific promoter comprises an nucleic acid having at least 90% sequence identity to SEQ ID No:28, SEQ ID No:30, SEQ ID No:32, SEQ ID No:34, SEQ ID No:36, SEQ ID No:38, SEQ ID No:40, SEQ ID No:42 and SEQ ID No:44.
5. The transgenic rice plant of any one of claims 1-4, wherein the nucleic acid sequence encoding κ-casein and/or the functional fragment thereof is codon-optimized.
6. The transgenic rice plant of any one of claims 1-4, wherein the nucleic acid sequence encoding β-casein and/or the functional fragment thereof is codon-optimized.
7. The transgenic rice plant of any one of claims 1-4, wherein the nucleic acid sequence encoding α-S1 casein and/or the functional fragment thereof is codon-optimized.
8. The transgenic rice plant of any one of claims 1-4, wherein the nucleic acid sequence encoding α-S2 casein and/or the functional fragment thereof is codon-optimized.
9. The transgenic rice plant of any one of claims 1-4, wherein the nucleic acid sequence encoding α-lactalbumin and/or the functional fragment thereof is codon-optimized.
10. The transgenic rice plant of any one of claims 1-4, wherein the nucleic acid sequence encoding β-lactoglobulin and/or the functional fragment thereof is codon-optimized.
11. The transgenic rice plant of any one of claims 1-4, wherein the nucleic acid sequence encoding lysozyme and/or the functional fragment thereof is codon-optimized.
12. The transgenic rice plant of any one of claims 1-5, wherein the nucleic acid sequence encodes κ-casein protein and/or the functional fragment thereof, having at least 90% sequence identity to SEQ ID No:5.
13. The transgenic rice plant of any one of claims 1-4 and 6, wherein the nucleic acid sequence encoding β-casein and/or the functional fragment thereof, having at least 90% sequence identity to SEQ ID No:7.
14. The transgenic rice plant of any one of claims 1-4 and 7, wherein the nucleic acid sequence encoding α-S1 casein and/or the functional fragment thereof, having at least 90% sequence identity to SEQ ID No:11.
15. The transgenic rice plant of any one of claims 1-4 and 8, wherein the nucleic acid sequence encoding α-S2 casein and/or the functional fragment thereof, having at least 90% sequence identity to SEQ ID No:12.
16. The transgenic rice plant of any one of claims 1-4 and 9, wherein the nucleic acid sequence encoding α-lactalbumin and/or the functional fragment, having at least 90% sequence identity to SEQ ID No:22.
17. The transgenic rice plant of any one of claims 1-4 and 10, wherein the nucleic acid sequence encoding β-lactoglobulin and/or the functional fragment thereof, having at least 90% sequence identity to SEQ ID No:23.
18. The transgenic rice plant of any one of claims 1-4 and 11, wherein the nucleic acid sequence encoding lysozyme and/or the functional fragment, having at least 90% sequence identity to SEQ ID No:24.
19. The transgenic rice plant of any one of claims 1-18, wherein the termination sequence is a NOS terminator.
20. The transgenic rice plant of any one of claims 1-19, wherein the bovine milk protein comprises α-S1 casein, α-S2 casein, β-casein, κ-casein, α-lactalbumin, β-lactoglobulin, and lysozyme.
21. The transgenic rice plant of any one of claims 1-20, wherein the bovine milk protein further comprises proteolytic product of α-S1 casein, α-S2 casein, β-casein, κ-casein, α-lactalbumin, β-lactoglobulin, and lysozyme.
22. The transgenic rice plant of any one of claims 1-21, wherein the bovine milk protein further comprises peptides produced by proteolysis of α-S1 casein, α-S2 casein, β-casein κ-casein, lactalbumin, β-lactoglobulin, and lysozyme.
23. A method of producing said transgenic rice plant of any one of claims 1-22, said method comprising the steps of:

(a) introducing at least one expression cassette capable of expressing a bovine milk protein into a rice plant, a part thereof, or a cell thereof,

(b) obtaining the transgenic rice plant, the part thereof, or the cell thereof, which stably expresses the bovine milk protein

(c) cultivating the transgenic rice plant, the part thereof, or the cell thereof,

(d) harvesting the transgenic rice plant, the part thereof, or the cell thereof.
24. A method of producing a bovine milk protein from said transgenic rice plant of any one of claims 1-22, said method comprising the steps of:

(a) extracting the bovine milk protein from the transgenic rice plant, the part thereof, or the cell thereof, and

(b) purifying the bovine milk protein from the transgenic rice plant, the part thereof, or the cell thereof;

wherein the bovine milk protein comprises α-S1 casein, α-S2 casein, β-casein, κ-casein, α-lactalbumin, β-lactoglobulin, and lysozyme; wherein the bovine milk protein further comprises proteolytic product of α-S1 casein, α-S2 casein, β-casein, κ-casein, α-lactalbumin, β-lactoglobulin, and lysozyme; and wherein the bovine milk protein further comprises peptides produced by proteolysis of α-S1 casein, α-S2 casein, β-casein, κ-casein, α-lactalbumin, β-lactoglobulin, and lysozyme.

### Duckweed

1. A transgenic duckweed plant comprising a recombinant DNA construct, said construct comprising
(i) a promoter,
(ii) a nucleic acid sequence encoding a bovine milk protein and/or a functional fragment thereof, which is operably linked to said promoter, and
(iii) a termination sequence;
   wherein the bovine milk protein and/or the functional fragment thereof is expressed in the transgenic duckweed plant and/or a part thereof; and wherein the bovine milk protein is selected from the group consisting of α-S1 casein, α-S2 casein, β-casein, κ-casein, α-lactalbumin, β-lactoglobulin, serum albumin, lactoferrin, lysozyme, lactoperoxidase, immunoglobulin-A, and lipase.
2. The transgenic duckweed plant of claim 1, wherein the promoter is selected from a Cauliflower mosaic virus (CaMV) 35S promoter, a plant constitutive promoter, and a plant tissue-specific promoter.
3. The transgenic duckweed plant of any one of claims 1-2, wherein the plant constitutive promoter comprises an nucleic acid having at least 90% sequence identity to SEQ ID No:46, SEQ ID No:47, SEQ ID No:48, and SEQ ID No:49.
4. The transgenic duckweed plant of any one of claims 1-3, wherein the plant tissue-specific promoter comprises an nucleic acid having at least 90% sequence identity to SEQ ID No:28, SEQ ID No:30, SEQ ID No:32, SEQ ID No:34, SEQ ID No:36, SEQ ID No:38, SEQ ID No:40, SEQ ID No:42 and SEQ ID No:44.
5. The transgenic duckweed plant of any one of claims 1-4, wherein the nucleic acid sequence encoding κ-casein and/or the functional fragment thereof is codon-optimized.
6. The transgenic duckweed plant of any one of claims 1-4, wherein the nucleic acid sequence encoding β-casein and/or the functional fragment thereof is codon-optimized.
7. The transgenic duckweed plant of any one of claims 1-4, wherein the nucleic acid sequence encoding α-S1 casein and/or the functional fragment thereof is codon-optimized.
8. The transgenic duckweed plant of any one of claims 1-4, wherein the nucleic acid sequence encoding α-S2 casein and/or the functional fragment thereof is codon-optimized.
9. The transgenic duckweed plant of any one of claims 1-4, wherein the nucleic acid sequence encoding α-lactalbumin and/or the functional fragment thereof is codon-optimized.
10. The transgenic duckweed plant of any one of claims 1-4, wherein the nucleic acid sequence encoding β-lactoglobulin and/or the functional fragment thereof is codon-optimized.
11. The transgenic duckweed plant of any one of claims 1-4, wherein the nucleic acid sequence encoding lysozyme and/or the functional fragment thereof is codon-optimized.
12. The transgenic duckweed plant of any one of claims 1-5, wherein the nucleic acid sequence encodes κ-casein protein and/or the functional fragment thereof, having at least 90% sequence identity to SEQ ID No:5.
13. The transgenic duckweed plant of any one of claims 1-4 and 6, wherein the nucleic acid sequence encoding β-casein and/or the functional fragment thereof, having at least 90% sequence identity to SEQ ID No:7.
14. The transgenic duckweed plant of any one of claims 1-4 and 7, wherein the nucleic acid sequence encoding α-S1 casein and/or the functional fragment thereof, having at least 90% sequence identity to SEQ ID No:11.
15. The transgenic duckweed plant of any one of claims 1-4 and 8, wherein the nucleic acid sequence encoding α-S2 casein and/or the functional fragment thereof, having at least 90% sequence identity to SEQ ID No:12.
16. The transgenic duckweed plant of any one of claims 1-4 and 9, wherein the nucleic acid sequence encoding α-lactalbumin and/or the functional fragment, having at least 90% sequence identity to SEQ ID No:22.
17. The transgenic duckweed plant of any one of claims 1-4 and 10, wherein the nucleic acid sequence encoding β-lactoglobulin and/or the functional fragment thereof, having at least 90% sequence identity to SEQ ID No:23.
18. The transgenic duckweed plant of any one of claims 1-4 and 11, wherein the nucleic acid sequence encoding lysozyme and/or the functional fragment, having at least 90% sequence identity to SEQ ID No:24.
19. The transgenic duckweed plant of any one of claims 1-18, wherein the termination sequence is a NOS terminator.
20. The transgenic duckweed plant of any one of claims 1-19, wherein the bovine milk protein comprises α-S1 casein, α-S2 casein, β-casein, κ-casein, α-lactalbumin, β-lactoglobulin, and lysozyme.
21. The transgenic duckweed plant of any one of claims 1-20, wherein the bovine milk protein further comprises proteolytic product of α-S1 casein, α-S2 casein, β-casein, κ-casein, α-lactalbumin, β-lactoglobulin, and lysozyme.
22. The transgenic duckweed plant of any one of claims 1-21, wherein the bovine milk protein further comprises peptides produced by proteolysis of α-S1 casein, α-S2 casein, β-casein κ-casein, lactalbumin, β-lactoglobulin, and lysozyme.
23. A method of producing said transgenic duckweed plant of any one of claims 1-22, said method comprising the steps of:

(a) introducing at least one expression cassette capable of expressing a bovine milk protein into a duckweed plant, a part thereof, or a cell thereof,

(b) obtaining the transgenic duckweed plant, the part thereof, or the cell thereof, which stably expresses the bovine milk protein

(c) cultivating the transgenic duckweed plant, the part thereof, or the cell thereof,

(d) harvesting the transgenic duckweed plant, the part thereof, or the cell thereof.
24. A method of producing a bovine milk protein from said transgenic duckweed plant of any one of claims 1-22, said method comprising the steps of:

(a) extracting the bovine milk protein from the transgenic duckweed plant, the part thereof, or the cell thereof, and

(b) purifying the bovine milk protein from the transgenic duckweed plant, the part thereof, or the cell thereof;

wherein the bovine milk protein comprises α-S1 casein, α-S2 casein, β-casein, κ-casein, α-lactalbumin, β-lactoglobulin, and lysozyme; wherein the bovine milk protein further comprises proteolytic product of α-S1 casein, α-S2 casein, β-casein, κ-casein, α-lactalbumin, β-lactoglobulin, and lysozyme; and wherein the bovine milk protein further comprises peptides produced by proteolysis of α-S1 casein, α-S2 casein, β-casein, κ-casein, α-lactalbumin, β-lactoglobulin, and lysozyme.

### INCORPORATION BY REFERENCE

All references, articles, publications, patents, patent publications, and patent applications cited anywhere herein, including above and below this section, are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

### REFERENCES

Swaisgood H. E., 1982, Chemistry of milk protein. In: Fox P. F. (ed.): Developments in Dairy Chemistry. Elsevier Applied Science Publishers, London, U K, 1-59.
Rodriquez et al., 1985, Effects of relative humidity, maximum and minimum temperature, pregnancy, and stage of lactation on milk composition and yield. Journal of Dairy Science, 68, 973-978.
Maas J., France J., McBride B. (1997): Model of milk protein synthesis. A mechanistic model of milk protein synthesis in the lactating bovine mammary gland. Journal of Theoretical Biology, 187, 363-378.
Elgar D. F., Norris C. S., Ayers J. S., Pritchard M., Otter D. E., Palmano K. P. (2000): Simultaneous separation and quantitation of the major bovine whey proteins including proteose peptone and caseinomacropeptide by reversed-phase high-performance liquid chromatography on polystyrene-divinylbenzene. Journal of Chromatography A, 878, 183-196.
Kamiński, S., Cieślińska, A., & Kostyra, E. (2007). Polymorphism of bovine beta-casein and its potential effect on human health. Journal of applied genetics, 48(3), 189-198.
Murray et al., 1989, Codon usage in plant genes. Nucleic Acids Res. 17, 477-498.
Campbell et al., 1990, Codon usage in higher plants, green algae, and cyanobacteria. Plant Physiol. 92, 1-11.
Horvath H. et al., 2000, The production of recombinant proteins in transgenic barley grains. Proc. Natl. Acad. Sci. USA, 97:1914-1919.
Jensen L. G. et al., 1996, Transgenic barley expressing a protein-engineered, thermostable (1,3-1,4)-beta-glucanase during germination, Proc. Natl. Acad. Sci. USA, 93:3487-3491. Patel et al, 2014 Milk Protein Concentrates: Manufacturing and Application. On-line publication
Patel H. et al, 2014, Technical Report: Milk Protein Concentrates: Manufacturing and Applications, www.usdairy.com/˜/media/usd/public/mpc-tech-report-final.pdf
Kinsella et al., 1984, Milk proteins: physicochemical and functional properties, CRC Crit. Rev. Food Sci. Nutr. 21:197-261.
Kinsella et al, 1989, Milk proteins: possible relationships of structure and function, in: Fox P. F. (Ed.), Developments in Dairy Chemistry-4-Functional Milk Proteins, Elsevier Appl. Sci., London, England, 1989, pp. 55-95.
Zhang N, McHale L K, Finer J J (2015) Isolation and characterization of “GmScream” promoters that regulate highly expressing soybean (Glycine max Merr.) genes. Plant Science 241:189-198.
De La Torre C M, Finer J J (2015) The intron and 5′ distal region of the soybean Gmubi promoter contribute to very high levels of gene expression in transiently and stably transformed tissues. Plant Cell Reports 34:111-120.
Kim, M. J., Kim, J. K., Kim, H. J., Pak, J. H., Lee, J. H., Kim, D. H., . . . & Ha, S. H. (2012). Genetic modification of the soybean to enhance the β-carotene content through seed-specific expression. PLoS One, 7(10), e48287.
Finer J J, M D McMullen (1991) Transformation of soybean via particle bombardment of embryogenic suspension culture tissue. In Vitro Cell and Develop Biol—Plant 27P:175-182.
Chiera, J. M., Bouchard, R A., Dorsey, S. L., Park, E., Buenrostro-Nava, M, T., Ling, P, P., & Finer, J. J. (2007). Isolation of two highly active soybean (Glycine max (L.) Men.) promoters and their characterization using a new automated image collection and analysis system. Plant Cell Reports, 26(9), 1501-1509.
Hernandez-Garcia, C. M., Martinelli, A. P., Bouchard, R A., & Finer, J. J. (2009). A soybean (Glycine max) polynbiquitin promoter gives strong constitutive expression in transgenic soybean. Plant cell reports, 28(5), 837-849,
Hernandez-Garcia, C. M., Bouchard, R. A., Rushton, P. J., Jones, M, L., Chen, X., Timko, M. P., & Finer, J. J. (2010). High level transgenic expression of soybean (Glycine max) GmEPF and Gmubi gene promoters isolated by a novel promoter analysis pipeline. BMC plant biology, 10(1), 237.
Jain R K, Joyce P B, Molinete M., Halban P A, and Gorr S U. (2001) Biochem. 1, 360, 645-649.
Snapp E L, Hegde R S, Francolini M., Lombardo F., Colombo S., Pedrazzini E., Borgese N., Lippincott-Schwartz J., (2003) J Cell Biol. October 27; 163(2):257-69.

1. A stably transformed dicotyledonous plant comprising in its genome a recombinant DNA construct, said construct comprising:
(i) a promoter,
(ii) a nucleic acid sequence encoding a bovine κ-casein milk protein having at least 90% sequence identity to SEQ ID NO:5, which is operably linked to said promoter, and
(iii) a termination sequence;
wherein the bovine milk protein is stably expressed in the plant and/or a part thereof in an amount of 1% or higher per the total protein weight of the soluble protein extractable from the plant or part thereof.

2. The stably transformed dicotyledonous plant of claim 1, wherein the plant is selected from the group consisting of soybean, lima bean, Arabidopsis, and tobacco.

3. The stably transformed dicotyledonous plant of claim 1, wherein the promoter is selected from a Cauliflower mosaic virus (CaMV) 35S promoter, a plant constitutive promoter, and a plant tissue-specific promoter.

4. The stably transformed dicotyledonous plant of claim 3, wherein the plant constitutive promoter comprises an nucleic acid having at least 90% sequence identity to SEQ ID No:49.

5. The stably transformed dicotyledonous plant of claim 3, wherein the plant tissue-specific promoter comprises an nucleic acid having at least 90% sequence identity to SEQ ID No:32.

6. The stably transformed dicotyledonous plant of claim 1, wherein the nucleic acid sequence encoding the bovine κ-casein milk protein is codon-optimized.

7-19. (canceled)

20. The stably transformed dicotyledonous plant of claim 1, wherein the termination sequence is a NOS terminator.

21.-22. (canceled)

23. The stably transformed dicotyledonous plant of claim 1, wherein the bovine milk protein further comprises peptides produced by proteolysis of κ-casein.

24. A method of producing the stably transformed dicotyledonous plant of claim 1, said method comprising the steps of:
(a) introducing at least one expression cassette capable of expressing a bovine κ-casein milk protein into a plant, a part thereof, or a cell thereof,
(b) obtaining the stably transformed dicotyledonous plant, the part thereof, or the cell thereof, which stably expresses the bovine κ-casein milk protein
(c) cultivating the stably transformed dicotyledonous plant, the part thereof, or the cell thereof,
(d) harvesting the stably transformed dicotyledonous plant, the part thereof, or the cell thereof.

25. A method of producing a bovine κ-casein milk protein from the stably transformed dicotyledonous plant of claim 1, said method comprising the steps of:
(a) extracting the bovine κ-casein milk protein from the stably transformed dicotyledonous plant, the part thereof, or the cell thereof, and
(b) purifying the bovine κ-casein milk protein from the stably transformed dicotyledonous plant, the part thereof, or the cell thereof.

26. A stably transformed dicotyledonous plant comprising in its genome a recombinant DNA construct, said construct comprising:
(i) a promoter,
(ii) a nucleic acid sequence encoding a bovine milk protein, which is operably linked to said promoter, and
(iii) a termination sequence;
wherein the bovine milk protein is stably expressed in the plant and/or a part thereof in an amount of 1% or higher per the total protein weight of the soluble protein extractable from the plant or part thereof; and
wherein the bovine milk protein is selected from the group consisting of α-S1 casein, α-S2 casein, β-casein, α-lactalbumin, β-lactoglobulin, and lysozyme.

27. The stably transformed dicotyledonous plant of claim 26, wherein the plant is selected from the group consisting of soybean, lima bean, Arabidopsis, and tobacco.

28. The stably transformed dicotyledonous plant of claim 26, wherein the promoter is selected from a Cauliflower mosaic virus (CaMV) 35S promoter, a plant constitutive promoter, and a plant tissue-specific promoter.

29. The stably transformed dicotyledonous plant of claim 28, wherein the plant constitutive promoter comprises a nucleic acid having at least 90% sequence identity to SEQ ID No:49.

30. The stably transformed dicotyledonous plant of claim 28, wherein the plant tissue-specific promoter comprises a nucleic acid having at least 90% sequence identity to SEQ ID No:32.

31. The stably transformed dicotyledonous plant of claim 26, wherein the nucleic acid sequence encoding the bovine milk protein is codon-optimized.

32. The stably transformed dicotyledonous plant of claim 26, wherein the bovine milk protein has a sequence with at least 90% sequence identity to any one of SEQ ID No:7, SEQ ID No:11, SEQ ID No:12, SEQ ID No:22, SEQ ID No:23, or SEQ ID No:24.

33. The stably transformed dicotyledonous plant of claim 26, wherein the termination sequence is a NOS terminator.

34. The stably transformed dicotyledonous plant of claim 26, wherein the bovine milk protein further comprises peptides produced by proteolysis of α-S1 casein, α-S2 casein, β-casein, α-lactalbumin, β-lactoglobulin, or lysozyme.

35. A method of producing the stably transformed dicotyledonous plant of claim 26, said method comprising the steps of:
(a) introducing at least one expression cassette capable of expressing a bovine milk protein into a plant, a part thereof, or a cell thereof,
(b) obtaining the stably transformed dicotyledonous plant, the part thereof, or the cell thereof, which stably expresses the bovine milk protein
(c) cultivating the stably transformed dicotyledonous plant, the part thereof, or the cell thereof,
(d) harvesting the stably transformed dicotyledonous plant, the part thereof, or the cell thereof.

36. A method of producing a bovine milk protein from the stably transformed dicotyledonous plant of claim 26, the method comprising the steps of:
(a) extracting the bovine milk protein from the stably transformed dicotyledonous plant, the part thereof, or the cell thereof, and
(b) purifying the bovine milk protein from the stably transformed dicotyledonous plant, the part thereof, or the cell thereof.
